(19) United States
(12) Patent Application Publication
(10) Pub. No.: US 2022/0281978 A1
(43) Pub. Date: Sep. 8, 2022

(54) Compositions and Methods for the Treatment of Cancer (Anti-ICOS Antibody Dosing)

(71) Applicant: Jounce Therapeutics, Inc., Cambridge, MA (US)

(72) Inventors: Jason Windham Reeves, Boston, MA (US); Deborah Law, Cambridge, MA (US); Christopher Harvey, Boston, MA (US); Elizabeth G. Trehu, Cambridge, MA (US); Lauren Pepper MacKenzie, Cambridge, MA (US); Amit Deshpande, Cambridge, MA (US); Jennifer S. Michaelson, Brighton, MA (US); Igor Feldman, Jamaica Plain, MA (US); Sriram Sathyanarayanan, Lexington, MA (US)

(73) Assignee: Jounce Therapeutics, Inc., Cambridge, MA (US)

(21) Appl. No.: 17/550,276

(22) Filed: Dec. 14, 2021

Related U.S. Application Data
(63) Continuation of application No. 16/497,937, filed on Sep. 26, 2019, now abandoned.
(60) Provisional application No. 62/514,591, filed on Jun. 2, 2017.
(60) Provisional application No. 62/481,025, filed on Apr. 3, 2017.

(51) Int. Cl. C07K 16/28; A61P 35/00; A61K 39/00; A61K 39/395; C12Q 1/6886; G01N 33/68

(52) U.S. Cl. CPC C07K 16/2818; A61P 35/00; A61K 39/0011; A61K 39/3955; C12Q 1/6886; G01N 33/6854; A61K 2039/505; A61K 2039/507; A61K 2039/545; C07K 2317/24; C07K 2317/565; C07K 2317/71; C07K 2317/72; C07K 2317/76; C07K 2317/92; C12Q 2600/106

(57) ABSTRACT

Methods of treating cancer with particular doses of anti-ICOS antibodies are provided.

### CROSS-REFERENCE TO RELATED APPLICATIONS

This application is a continuation of U.S. application Ser. No. 16/497,937, filed Sep. 26, 2019, which is a national phase entry pursuant to 35 U.S.C. § 371 of International Application No. PCT/US2018/025623, filed Apr. 2, 2018, which claims the benefit of priority of U.S. Provisional Application No. 62/481,025, filed Apr. 3, 2017, and U.S. Provisional Application No. 62/514,591, filed Jun. 2, 2017, each of which is incorporated by reference herein in its entirety for any purpose.

### SEQUENCE LISTING

This application contains a Sequence Listing, which has been submitted electronically in ASCII format and is hereby incorporated by reference in its entirety. Said ASCII copy, created on Mar. 13, 2018, is named 2018-03-13_01140-0007-00PCT_SEQLIST_ST25.txt and is 61,772 bytes in size.

### FIELD OF THE INVENTION

Methods of treating cancer with particular doses of anti-ICOS antibody are provided.

### BACKGROUND

ICOS is a member of the B7/CD28/CTLA-4 immunoglobulin superfamily and is specifically expressed on T cells. Unlike CD28, which is constitutively expressed on T cells and provides co-stimulatory signals necessary for full activation of resting T cells, ICOS is expressed only after initial T cell activation.

ICOS has been implicated in diverse aspects of T cell responses (reviewed in Simpson et al., 2010, Curr. Opin. Immunol., 22: 326-332). It plays a role in the formation of germinal centers, TB cell collaboration, and immunoglobulin class switching. ICOS-deficient mice show impaired germinal center formation and have decreased production of interleukin IL-10. These defects have been specifically linked to deficiencies in T follicular helper cells.

ICOS also plays a role in the development and function of other T cell subsets, including Th1, Th2, and Th17. Notably, ICOS co-stimulates T cell proliferation and cytokine secretion associated with both Th1 and Th2 cells. Accordingly, ICOS KO mice demonstrate impaired development of autoimmune phenotypes in a variety of disease models, including diabetes (Th1), airway inflammation (Th2) and EAE neuro-inflammatory models (Th17).

In addition to its role in modulating T effector (Teff) cell function, ICOS also modulates T regulatory cells (Tregs). ICOS is expressed at high levels on Tregs, and has been implicated in Treg homeostasis and function.

Upon activation, ICOS, a disulfide-linked homodimer, induces a signal through the PI3K and AKT pathways. Subsequent signaling events result in expression of lineage specific transcription factors (e.g., T-bet, GATA-3) and, in turn, effects on T cell proliferation and survival.

ICOS ligand (ICOSL; B7-H2; B7RP1; CD275; GL50), also a member of the B7 superfamily, is the only ligand for ICOS and is expressed on the cell surface of B cells, macrophages and dendritic cells. ICOSL functions as a non-covalently linked homodimer on the cell surface in its interaction with ICOS. Human ICOSL, although not mouse ICOSL, has been reported to bind to human CD28 and CTLA-4 (Yao et al., 2011, Immunity, 34: 729-740).

### SUMMARY

Embodiment 1. A method of treating cancer in a subject, comprising administering a dose of 0.3 mg/kg of an anti-ICOS antibody to said subject, wherein said anti-ICOS antibody comprises an HCDR1 comprising the amino acid sequence of SEQ ID NO: 62; an HCDR2 comprising the amino acid sequence of SEQ ID NO: 63; an HCDR3 comprising the amino acid sequence of SEQ ID NO: 64; an LCDR1 comprising the amino acid sequence of SEQ ID NO: 65; an LCDR2 comprising the amino acid sequence of SEQ ID NO: 66; and an LCDR3 comprising the amino acid sequence of SEQ ID NO: 67.

Embodiment 2. The method of embodiment 1, wherein said dose is administered once every three weeks.

Embodiment 3. The method of embodiment 1, wherein said dose is administered once every four weeks.

Embodiment 4. The method of embodiment 1, wherein said dose is administered once every six weeks.

Embodiment 5. A method of treating cancer in a subject, comprising administering a dose of 0.1 mg/kg of an anti-ICOS antibody to said subject, wherein said anti-ICOS antibody comprises an HCDR1 comprising the amino acid sequence of SEQ ID NO: 62; an HCDR2 comprising the amino acid sequence of SEQ ID NO: 63; an HCDR3 comprising the amino acid sequence of SEQ ID NO: 64; an LCDR1 comprising the amino acid sequence of SEQ ID NO: 65; an LCDR2 comprising the amino acid sequence of SEQ ID NO: 66; and an LCDR3 comprising the amino acid sequence of SEQ ID NO: 67.

Embodiment 6. The method of embodiment 5, wherein said dose is administered once ever three weeks.

Embodiment 7. The method of embodiment 5, wherein said dose is administered once every four weeks.

Embodiment 8. The method of embodiment 5, wherein said dose is administered once every six weeks.

Embodiment 9. The method of any one of embodiments 1-8, wherein, prior to said administering, said method further comprises selecting said subject for treatment with said anti-ICOS antibody.

Embodiment 10. The method of embodiment 9, wherein said selecting comprises:
a) detecting the levels of at least two, at least three, at least four, at least five, at least six, at least seven, at least eight, at least nine, or at least ten mRNAs selected from the mRNAs in Table 7 in a sample from a subject; and
b) if the level of at least one, at least two, at least three, at least four, at least five, at least six, at least seven, at least eight, at least nine, or at least ten of the mRNAs is above a threshold level, then selecting said subject for treatment with said anti-ICOS antibody.

Embodiment 11. The method of embodiment 10, wherein the threshold level is determined relative to a reference mRNA.

Embodiment 12. The method of embodiment 11, wherein the reference mRNA is a housekeeping mRNA.

Embodiment 13. The method of any one of embodiments 10-12, wherein the method comprises detecting the levels of at least two, at least three, at least four, at least five, at least six, at least seven, at least eight, at least nine, or at least ten mRNAs selected from CCRS, CD2, CD96, CTLA4, CXCR6, FOXP3, ICOS, ITK, P2RY10, SIRPG, and TIGIT.

Embodiment 14. The method of any one of embodiments 10-13, wherein the detecting comprises at least one method selected from amplification and hybridization.

Embodiment 15. The method of embodiment 14, wherein the method comprises quantitative PCR.

Embodiment 16. The method of embodiment 14, wherein the method comprises hybridization on an array.

Embodiment 17. The method of any one embodiments 10-16, wherein the sample is a cancer sample.

Embodiment 18. The method of embodiment 9, wherein said selecting comprises contacting T cells from said subject with a test agonist anti-ICOS antibody and determining whether NKp46 ligand (NKp46-L) is induced on the T cells wherein if NKp46-L is induced on the T cells, the subject is selected for treatment with said anti-ICOS agonist antibody.

Embodiment 19. The method of embodiment 9, wherein said selecting comprises detecting the level of ICOS in a sample from the subject.

Embodiment 20. The method of embodiment 19, wherein the detecting comprises immunohistochemistry.

Embodiment 21. The method of embodiment 20, wherein immunohistochemistry comprises contacting the sample with an antibody selected from:
(i) an antibody comprising (a) HCDR1 comprising the amino acid sequence of SEQ ID NO: 194; (b) HCDR2 comprising the amino acid sequence of SEQ ID NO: 195; (c) HCDR3 comprising the amino acid sequence of SEQ ID NO: 196; (d) LCDR1 comprising the amino acid sequence of SEQ ID NO: 197; (e) LCDR2 comprising the amino acid sequence of SEQ ID NO: 198; and (f) LCDR3 comprising the amino acid sequence of SEQ ID NO: 199; or
(ii) an antibody comprising (a) HCDR1 comprising the amino acid sequence of SEQ ID NO: 202; (b) HCDR2 comprising the amino acid sequence of SEQ ID NO: 203; (c) HCDR3 comprising the amino acid sequence of SEQ ID NO: 204; (d) LCDR1 comprising the amino acid sequence of SEQ ID NO: 205; (e) LCDR2 comprising the amino acid sequence of SEQ ID NO: 206; and (f) LCDR3 comprising the amino acid sequence of SEQ ID NO: 207; or
(iii) an antibody comprising (a) HCDR1 comprising the amino acid sequence of SEQ ID NO: 210; (b) HCDR2 comprising the amino acid sequence of SEQ ID NO: 211; (c) HCDR3 comprising the amino acid sequence of SEQ ID NO: 212; (d) LCDR1 comprising the amino acid sequence of SEQ ID NO: 213; (e) LCDR2 comprising the amino acid sequence of SEQ ID NO: 214; and (f) LCDR3 comprising the amino acid sequence of SEQ ID NO: 215.

Embodiment 22. The method of embodiment 21, wherein the antibody is selected from:
(i) an antibody comprising a VH that is at least 90%, 91%, 92%, 93%, 94%, 95%, 96%, 97%, 98%, 99%, or 100% identical to the amino acid sequence of SEQ ID NO: 192 and a VL that is at least 90%, 91%, 92%, 93%, 94%, 95%, 96%, 97%, 98%, 99%, or 100% identical to the amino acid sequence of SEQ ID NO: 193; or
(ii) an antibody comprising a VH that is at least 90%, 91%, 92%, 93%, 94%, 95%, 96%, 97%, 98%, 99%, or 100% identical to the amino acid sequence of SEQ ID NO: 200 and a VL that is at least 90%, 91%, 92%, 93%, 94%, 95%, 96%, 97%, 98%, 99%, or 100% identical to the amino acid sequence of SEQ ID NO: 201; or
(iii) an antibody comprising a VH that is at least 90%, 91%, 92%, 93%, 94%, 95%, 96%, 97%, 98%, 99%, or 100% identical to the amino acid sequence of SEQ ID NO: 208 and a VL that is at least 90%, 91%, 92%, 93%, 94%, 95%, 96%, 97%, 98%, 99%, or 100% identical to the amino acid sequence of SEQ ID NO: 209.

Embodiment 23. The method of embodiment 21 or embodiment 22, wherein the antibody is selected from:
(i) an antibody comprising a VH comprising the amino acid sequence of SEQ ID NO: 192 and a VL comprising the amino acid sequence of SEQ ID NO: 193; or
(ii) an antibody comprising a VH comprising the amino acid sequence of SEQ ID NO: 200 and a VL comprising the amino acid sequence of SEQ ID NO: 201; or
(iii) an antibody comprising a VH comprising the amino acid sequence of SEQ ID NO: 208 and a VL comprising the amino acid sequence of SEQ ID NO: 209.

Embodiment 24. The method of any one of embodiments 19 to 23, wherein the sample is a tumor sample.

Embodiment 25. The method of any one of the preceding embodiments, wherein the subject has a cancer selected from melanoma, non-small cell lung cancer (NSCLC), renal cell carcinoma (RCC) (e.g., clear cell RCC), gastric cancer, bladder cancer, endometrial cancer, MSI-H cancer of any organ, diffuse large B-cell lymphoma (DLBCL), Hodgkin's lymphoma, ovarian cancer (e.g., endometrioid ovarian cancer), head & neck squamous cell cancer (HNSCC), acute myeloid leukemia (AML), rectal cancer, refractory testicular cancer, small cell lung cancer (SCLC), small bowel cancer, metastatic cutaneous squamous cell cancer, cervical cancer, MSI-high colon cancer, esophageal cancer, mesothelioma, breast cancer, and triple negative breast cancer (TNBC).

Embodiment 26. The method of any one of the preceding embodiments, wherein the subject has a cancer selected from melanoma, gastric cancer, endometrial cancer, MSI-H cancers of any organ, head & neck squamous cell cancer (HNSCC), non-small cell lung cancer (NSCLC), and triple negative breast cancer (TNBC).

Embodiment 27. The method of any one of the preceding embodiments, wherein said anti-ICOS antibody binds to human ICOS, and wherein the antibody also binds to mouse ICOS and/or rat ICOS.

Embodiment 28. The method of embodiment 27, wherein the antibody binds to human ICOS with an affinity (K_{D}) of less than 5 nM.

Embodiment 29. The method of embodiment 28, wherein affinity is determined using biolayer interferometry.

Embodiment 30. The method of any one of the preceding embodiments, wherein the anti-ICOS antibody comprises a heavy chain variable region (VH) and a light chain variable region (VL), wherein the VH is at least 90%, 91%, 92%, 93%, 94%, 95%, 96%, 97%, 98%, 99%, or 100% identical to the amino acid sequence of SEQ ID NO: 60 and the VL is at least 90%, 91%, 92%, 93%, 94%, 95%, 96%, 97%, 98%, 99%, or 100% identical to the amino acid sequence of SEQ ID NO: 61.

Embodiment 31. The method of embodiment 30, wherein said VH comprises the amino acid sequence of SEQ ID NO: 60 and said VL comprises the amino acid sequence of SEQ ID NO: 61.

Embodiment 32. The method of any one of the preceding embodiments, wherein the anti-ICOS antibody is a monoclonal antibody.

Embodiment 33. The method of embodiment 32, wherein the anti-ICOS antibody is a humanized antibody.

Embodiment 34. The method of any one the preceding embodiments, wherein the anti-ICOS antibody is a full length antibody.

Embodiment 35. The method of any one of the preceding embodiments, wherein the anti-ICOS antibody comprises a heavy chain comprising the amino acid sequence of SEQ ID NO: 188 and a light chain comprising the amino acid sequence of SEQ ID NO: 189.

Embodiment 36. The method of embodiment 29, wherein the anti-ICOS antibody consists of a heavy chain having the amino acid sequence of SEQ ID NO: 188 and a light chain having the amino acid sequence of SEQ ID NO: 189.

Embodiment 37. The method of any one of embodiments 1-34, wherein the anti-ICOS antibody comprises a heavy chain comprising the amino acid sequence of SEQ ID NO: 216 and a light chain comprising the amino acid sequence of SEQ ID NO: 189.

Embodiment 38. The method of any one of embodiments 1-34, wherein the anti-ICOS antibody consists of a heavy chain having the amino acid sequence of SEQ ID NO: 216 and a light chain having the amino acid sequence of SEQ ID NO: 189.

Embodiment 39. The method of any one of the preceding embodiments, wherein administration of the anti-ICOS antibody to a mammal results in an increase in T effector (Teff) cells in the mammal.

Embodiment 40. The method of any one of the preceding embodiments, wherein administration of the antibody to a mammal results in activation of T effector (Teff) cells in the mammal.

Embodiment 41. The method of embodiment 39 or embodiment 40, wherein the Teff cells are CD4+ FoxP3− T cells.

Embodiment 42. The method of embodiment 39 or embodiment 40, wherein the Teff cells are CD4+ FoxP3− T cells and CD8+ T cells.

Embodiment 43. The method of embodiment 39 or embodiment 40, wherein the Teff cells are CD8+ T cells.

Embodiment 44. The method of any one of the preceding embodiments, wherein administration of the antibody to said subject results in a decrease in T regulatory (Treg) cells in said subject.

Embodiment 45. The method of embodiment 44, wherein the Treg cells are CD4+ FoxP3+ T cells.

Embodiment 46. The method of any one of the preceding embodiments, wherein the subject is a human.

Embodiment 47. The method of any one of the preceding embodiments, wherein the method comprises administering an anti-ICOS antibody and at least one additional therapeutic agent.

Embodiment 48. The method of embodiment 47, wherein the additional therapeutic agent is administered concurrently or sequentially with the anti-ICOS antibody.

Embodiment 49. The method of embodiment 47 or embodiment 48, wherein the additional therapeutic agent is selected from an anti-PD-1 antibody and an anti-PD-L1 antibody.

Embodiment 50. The method of embodiment 49, wherein the additional therapeutic agent is an anti-PD-1 antibody.

Embodiment 51. The method of embodiment 50, wherein the anti-PD-1 antibody is nivolumab.

Embodiment 52. The method of embodiment 50 or embodiment 51, wherein the anti-PD-1 antibody is administered at a flat dose of 240 mg.

Embodiment 53. The method of embodiment 47 or claim 48, wherein the additional therapeutic agent is an anti-CTLA4 antibody.

Embodiment 54. The method of embodiment 53, wherein the anti-CTLA4 antibody is ipilimumab or tremelimumab.

Embodiment 55. The method of embodiment 47 or embodiment 48, wherein the additional therapeutic agent is a cancer vaccine.

Embodiment 56. The method of embodiment 55, wherein the cancer vaccine is selected from a DNA vaccine, an engineered virus vaccine, an engineered tumor cell vaccine, and a cancer vaccine developed using neoantigens.

Embodiment 57. An isolated anti-ICOS antibody, wherein said antibody comprises a heavy chain comprising the amino acid sequence of SEQ ID NO: 216 and a light chain comprising the amino acid sequence of SEQ ID NO: 189.

Embodiment 58. An isolated anti-ICOS antibody, wherein said antibody comprises a heavy chain consisting of the amino acid sequence of SEQ ID NO: 216 and a light chain consisting of the amino acid sequence of SEQ ID NO: 189.

In certain embodiments, the invention comprises administering an anti-ICOS antibody and at least one additional therapeutic agent. In some embodiments, the additional therapeutic agent is administered concurrently or sequentially with the anti-ICOS antibody. In some embodiments, the additional therapeutic agent is a PD-1 therapy. In some embodiments, the additional therapeutic agent is selected from an anti-PD-1 antibody and an anti-PD-L1 antibody. In some embodiments, an anti-ICOS antibody provided herein is administered with nivolumab. In some embodiments, nivolumab is administered at a flat dose of 240 mg. In some embodiments, an anti-ICOS antibody provided herein is administered with pembrolizumab. In some embodiments, an anti-ICOS antibody provided herein is administered with atezolizumab. In some embodiments, an anti-ICOS antibody provided herein is administered with avelumab. In some embodiments, an anti-ICOS antibody provided herein is administered with durvalumab.

In some embodiments, the additional therapeutic agent is a cancer vaccine. In some embodiments, the cancer vaccine is selected from a DNA vaccine, an engineered virus vaccine, an engineered tumor cell vaccine, and a cancer vaccine developed using neoantigens.

In some embodiments, the anti-ICOS antibody provided herein is administered with an agonist anti-OX40 antibody. In some embodiments, the anti-ICOS antibody provided herein is administered with an anti-CTLA4 antibody. In some embodiments, the anti-ICOS antibody provided herein is administered with ipilimumab.

In some embodiments, the additional therapeutic is a chemotherapeutic agent.

Nonlimiting exemplary chemotherapeutic agents include capecitabine, cyclophosphamide, dacarbazine, temozolomide, cyclophosphamide, docetaxel, doxorubicin, daunorubicin, cisplatin, carboplatin, epirubicin, eribulin, 5-FU, gemcitabine, irinotecan, ixabepilone, methotrexate, mitoxantrone, oxaliplatin, paclitaxel, nab-paclitaxel, ABRAXANE® (protein-bound paclitaxel), pemetrexed, vinorelbine, and vincristine. In some embodiments, the anti-ICOS antibody provided herein is administered with ABRAXANE® (Celgene). In some embodiments, an anti-ICOS antibody provided herein is administered with at least one kinase inhibitor. Nonlimiting exemplary kinase inhibitors include erlotinib, afatinib, gefitinib, crizotinib, dabrafenib, trametinib, vemurafenib, and cobimetanib.

In some embodiments, the additional therapeutic agent is an IDO inhibitor. Nonlimiting exemplary IDO inhibitors include indoximod (New Link Genetics), epicadostat (Incyte Corp), 1-methyl-D-tryptophan (New Link Genetics), and GDC-0919 (Genentech). In some embodiments, the additional therapeutic agent is an immune-modifying drug (IMiD). Nonlimiting exemplary IMiDs include thalidomide, lenalidomide, and pomalidomide.

In some embodiments, the subject receives CAR-T therapy in addition to administration of anti-ICOS an antibody described herein.

In some embodiments, the mammal undergoes surgery and/or radiation therapy in addition to administration of an anti-ICOS antibody described herein, with or without an additional therapeutic agent. In some embodiments, the mammal undergoes radiation therapy in addition to administration of anti-ICOS an antibody described herein, with or without an additional therapeutic agent.

### BRIEF DESCRIPTION OF THE DRAWINGS

FIG. 1A is a graph showing average tumor volume in mice treated with the indicated dose of 37A10S713M.

FIG. 1B is a graph showing percent ICOS available in peripheral blood of mice treated with the indicated dose at the indicated time points.

FIG. 1C is a table summarizing the number of tumor free mice resulting from each of the three doses.

FIG. 1D is a series of graphs showing tumor volume as a function of time in individual mice treated at the indicated doses.

FIG. 2A is a schematic showing a dosing and collection schedule for the PK/RA/Lymphocyte phenotyping arm of a mouse study (RA=receptor availability).

FIGS. 2B-1 and 2B-2 are a series of graphs showing concentration of circulating treatment antibody at the indicated times.

FIG. 2C is a graph showing absorbance that measures anti-drug antibodies present in the serum of the indicated treated mice.

FIG. 2D is a series of graphs showing percentage of ICOS available in the tumors and peripheral blood in the indicated mice treated for the indicated periods of time after a first dose.

FIG. 3 is a series of graphs showing ICOS availability in non-human primates treated with 37A10S713 at the indicated doses following a first and/or second dose.

FIG. 4A is a graph showing simulations for predicted human plasma 37A10S713 concentration resulting from the indicated doses.

FIG. 4B is a graph showing predicted target engagement of ICOS as measured by free receptor in peripheral blood in subjects treated with 37A10S713.

FIG. 4C is a table showing the predicted serum/pasma concentrations of antibody for the indicated doses (mg/kg) based on the modeling of FIG. 4A.

FIG. 5 is a graph showing the concentration at the indicated times of therapeutic antibodyin the serum of human patients treated with the indicated dose of the therapeutic antibody.

FIG. 6 is a graph showing target engagement of two patients treated at 0.3 mg/kg at the indicated times.

FIGS. 7A and 7B shows graphs of mean levels of IFN-γ by dose of 37A10S713 in phase 1 study participants receiving 37A10S713 monotherapy (FIG. 7A) and 37A10S713+ nivolumab combination therapy (FIG. 7B).

### DETAILED DESCRIPTION OF SOME EMBODIMENTS

In general, the invention features a method of treating cancer by administering an anti-ICOS agonist antibody (e.g., antibody 37A10S713, described below) at a particular dose. Specifically, in some embodiments, the method comprises administering a dose of either 0.1 mg/kg or 0.3 mg/kg (administered, e.g., once every three, four, or six weeks). Based in part on the preclinical data disclosed herein, criteria were established for evaluating preliminary human clinical data, obtained over a range of doses, in order to identify a dose likely to be both safe and efficacious for the treatment of cancer in certain subjects. Accordingly, the first criteria was that the dose of anti-ICOS agonist antibody be observed to be sufficiently safe in humans based on the absence of dose limiting toxicity events at the selected dose. The second criteria was that a desired dose of an anti-ICOS agonist antibody (e.g., antibody 37A10S713) achieve near complete or complete engagement with ICOS (i.e., target engagement or TE, which is assessed in some embodiments using a target availability assay), in vivo, for an extended period of time (e.g., 8, 15 or 21 days). This criteria was based, in part, on the preclinical observation that such target engagement properties appeared to correlate with efficacy. In order to minimize depletion of beneficial immune cell populations, and potential related toxicity, the third criteria was that a desired dose would not demonstrate any significant change in peripheral T lymphocytes or subsets thereof, such as T effector cells and/or T regulatory cells. Thus, the present invention is based, in part, on the following discoveries related to the administration of an anti-ICOS agonist antibody (e.g., antibody 37A10S713) to human subjects: (1) doses between 0.1 mg/kg and 0.3 mg/kg (e.g., a dose of 0.1 mg/kg or 0.3 mg/kg administered, e.g., once every three, four, or six weeks) were not observed to result in dose limiting toxicity events in humans; (2) doses between 0.1 mg/kg and 0.3 mg/kg (e.g., a dose of 0.1 mg/kg or 0.3 mg/kg administered, e.g., once every three, four, or six weeks) show target engagement levels and durations consistent with the above criteria for efficacy (>90% target engagement through day 21 following the first dose); (3) doses that are substantially lower than 0.1 mg/kg show serum concentrations indicative of target engagement levels and/or durations inconsistent with the above criteria for efficacy; and (4) doses between 0.1 mg/kg and 0.3 mg/kg were found not to deplete peripheral T cells.

Gene expression signatures for predicting or determining ICOS expression, e.g., in a tumor environment, are provided. In some embodiments, the gene expression signatures may be used to identify patients who are likely to respond to anti-ICOS antibody therapy. In some embodiments, a gene expression signature comprising two or more genes in place of, or in addition to, ICOS may provide a more robust assay for ICOS expression than detecting ICOS alone. In some embodiments, methods of treatment are provided, comprising identifying a patient who is likely to respond to anti-ICOS antibody therapy using a gene expression signature described herein, and administering an anti-ICOS antibody.

The section headings used herein are for organizational purposes only and are not to be construed as limiting the subject matter described.

All references cited herein, including patent applications, patent publications, and Genbank Accession numbers are herein incorporated by reference, as if each individual reference were specifically and individually indicated to be incorporated by reference in its entirety.

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3rd. edition (2001) Cold Spring Harbor Laboratory Press, Cold Spring Harbor, N.Y. CURRENT PROTOCOLS IN MOLECULAR BIOLOGY (F. M. Ausubel, et al. eds., (2003)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993); and updated versions thereof.

### I. Definitions

Unless otherwise defined, scientific and technical terms used in connection with the present disclosure shall have the meanings that are commonly understood by those of ordinary skill in the art. Further, unless otherwise required by context or expressly indicated, singular terms shall include pluralities and plural terms shall include the singular. For any conflict in definitions between various sources or references, the definition provided herein will control.

It is understood that embodiments of the invention described herein include “consisting” and/or “consisting essentially of” embodiments. As used herein, the singular form “a”, “an”, and “the” includes plural references unless indicated otherwise. Use of the term “or” herein is not meant to imply that alternatives are mutually exclusive.

In this application, the use of “or” means “and/or” unless expressly stated or understood by one skilled in the art. In the context of a multiple dependent claim, the use of “or” refers back to more than one preceding independent or dependent claim.

As is understood by one skilled in the art, reference to “about” a value or parameter herein includes (and describes) embodiments that are directed to that value or parameter per se. For example, description referring to “about X” includes description of “X”.

The terms “nucleic acid molecule”, “nucleic acid” and “polynucleotide” may be used interchangeably, and refer to a polymer of nucleotides. Such polymers of nucleotides may contain natural and/or non-natural nucleotides, and include, but are not limited to, DNA, RNA, and PNA. “Nucleic acid sequence” refers to the linear sequence of nucleotides that comprise the nucleic acid molecule or polynucleotide.

The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

“ICOS” and “inducible T-cell costimulatory” as used herein refer to any native ICOS that results from expression and processing of ICOS in a cell. The term includes ICOS from any vertebrate source, including mammals such as primates (e.g., humans and cynomolgus monkeys) and rodents (e.g., mice and rats), unless otherwise indicated. The term also includes naturally occurring variants of ICOS, e.g., splice variants or allelic variants. The amino acid sequence of an exemplary human ICOS precursor protein, with signal sequence (with signal sequence, amino acids 1-20) is shown in SEQ ID NO: 1. The amino acid sequence of an exemplary mature human ICOS is shown in SEQ ID NO: 2. The amino acid sequence of an exemplary mouse ICOS precursor protein, with signal sequence (with signal sequence, amino acids 1-20) is shown in SEQ ID NO: 3. The amino acid sequence of an exemplary mature mouse ICOS is shown in SEQ ID NO: 4. The amino acid sequence of an exemplary rat ICOS precursor protein, with signal sequence (with signal sequence, amino acids 1-20) is shown in SEQ ID NO: 190. The amino acid sequence of an exemplary mature rat ICOS is shown in SEQ ID NO: 191. The amino acid sequence of an exemplary cynomolgus monkey ICOS precursor protein, with signal sequence (with signal sequence, amino acids 1-20) is shown in SEQ ID NO: 5. The amino acid sequence of an exemplary mature cynomolgus monkey ICOS is shown in SEQ ID NO: 6.

The term “specifically binds” to an antigen or epitope is a term that is well understood in the art, and methods to determine such specific binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to an ICOS epitope is an antibody that binds this epitope with greater affinity, avidity, more readily, and/or with greater duration than it binds to other ICOS epitopes or non-ICOS epitopes. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding. “Specificity” refers to the ability of a binding protein to selectively bind an antigen.

As used herein, “substantially pure” refers to material which is at least 50% pure (that is, free from contaminants), more preferably, at least 90% pure, more preferably, at least 95% pure, yet more preferably, at least 98% pure, and most preferably, at least 99% pure.

As used herein, the term “epitope” refers to a site on a target molecule (for example, an antigen, such as a protein, nucleic acid, carbohydrate or lipid) to which an antigen-binding molecule (for example, an antibody, antibody fragment, or scaffold protein containing antibody binding regions) binds. Epitopes often include a chemically active surface grouping of molecules such as amino acids, polypeptides or sugar side chains and have specific three-dimensional structural characteristics as well as specific charge characteristics. Epitopes can be formed both from contiguous and/or juxtaposed noncontiguous residues (for example, amino acids, nucleotides, sugars, lipid moiety) of the target molecule. Epitopes formed from contiguous residues (for example, amino acids, nucleotides, sugars, lipid moiety) typically are retained on exposure to denaturing solvents whereas epitopes formed by tertiary folding typically are lost on treatment with denaturing solvents. An epitope may include but is not limited to at least 3, at least 5 or 8-10 residues (for example, amino acids or nucleotides). In some examples an epitope is less than 20 residues (for example, amino acids or nucleotides) in length, less than 15 residues or less than 12 residues. Two antibodies may bind the same epitope within an antigen if they exhibit competitive binding for the antigen. In some embodiments, an epitope can be identified by a certain minimal distance to a CDR residue on the antigen-binding molecule. In some embodiments, an epitope can be identified by the above distance, and further limited to those residues involved in a bond (for example, a hydrogen bond) between an antibody residue and an antigen residue. An epitope can be identified by various scans as well, for example an alanine or arginine scan can indicate one or more residues that the antigen-binding molecule can interact with. Unless explicitly denoted, a set of residues as an epitope does not exclude other residues from being part of the epitope for a particular antibody. Rather, the presence of such a set designates a minimal series (or set of species) of epitopes. Thus, in some embodiments, a set of residues identified as an epitope designates a minimal epitope of relevance for the antigen, rather than an exclusive list of residues for an epitope on an antigen.

A “nonlinear epitope” or “conformational epitope” comprises noncontiguous polypeptides, amino acids and/or sugars within the antigenic protein to which an antibody specific to the epitope binds. In some embodiments, at least one of the residues will be noncontiguous with the other noted residues of the epitope; however, one or more of the residues can also be contiguous with the other residues.

A “linear epitope” comprises contiguous polypeptides, amino acids and/or sugars within the antigenic protein to which an antibody specific to the epitope binds. It is noted that, in some embodiments, not every one of the residues within the linear epitope need be directly bound (or involved in a bond) with the antibody. In some embodiments, linear epitopes can be from immunizations with a peptide that effectively consisted of the sequence of the linear epitope, or from structural sections of a protein that are relatively isolated from the remainder of the protein (such that the antibody can interact, at least primarily), just with that sequence section.

The term “antibody” herein is used in the broadest sense and encompasses various antibody structures, including but not limited to monoclonal antibodies, polyclonal antibodies, multispecific antibodies (for example, bispecific (such as Bi-specific T-cell engagers) and trispecific antibodies), and antibody fragments so long as they exhibit the desired antigen-binding activity.

The term antibody includes, but is not limited to, fragments that are capable of binding to an antigen, such as Fv, single-chain Fv (scFv), Fab, Fab′, di-scFv, sdAb (single domain antibody) and (Fab′)₂ (including a chemically linked F(ab′)2). Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen. The term antibody also includes, but is not limited to, chimeric antibodies, humanized antibodies, and antibodies of various species such as mouse, human, cynomolgus monkey, etc. Furthermore, for all antibody constructs provided herein, variants having the sequences from other organisms are also contemplated. Thus, if a human version of an antibody is disclosed, one of skill in the art will appreciate how to transform the human sequence based antibody into a mouse, rat, cat, dog, horse, etc. sequence. Antibody fragments also include either orientation of single chain scFvs, tandem di-scFv, diabodies, tandem tri-sdcFv, minibodies, etc. Antibody fragments also include nanobodies (sdAb, an antibody having a single, monomeric domain, such as a pair of variable domains of heavy chains, without a light chain). An antibody fragment can be referred to as being a specific species in some embodiments (for example, human scFv or a mouse scFv). This denotes the sequences of at least part of the non-CDR regions, rather than the source of the construct.

The term “monoclonal antibody” refers to an antibody of a substantially homogeneous population of antibodies, that is, the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. Thus, a sample of monoclonal antibodies can bind to the same epitope on the antigen. The modifier “monoclonal” indicates the character of the antibody as being obtained from a substantially homogeneous population of antibodies, and is not to be construed as requiring production of the antibody by any particular method. For example, the monoclonal antibodies may be made by the hybridoma method first described by Kohler and Milstein, 1975, Nature 256:495, or may be made by recombinant DNA methods such as described in U.S. Pat. No. 4,816,567. The monoclonal antibodies may also be isolated from phage libraries generated using the techniques described in McCafferty et al., 1990, Nature 348:552-554, for example.

The term “CDR” denotes a complementarity determining region as defined by at least one manner of identification to one of skill in the art. In some embodiments, CDRs can be defined in accordance with any of the Chothia numbering schemes, the Kabat numbering scheme, a combination of Kabat and Chothia, the AbM definition, the contact definition, and/or a combination of the Kabat, Chothia, AbM, and/or contact definitions. Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3. (Kabat et al., Sequences of Proteins of Immunological Interest, 5th Ed. Public Health Service, National Institutes of Health, Bethesda, Md. (1991)). The AbM definition can include, for example, CDRs (CDR-L1, CDR-L2, CDR-L3, CDR-H1, CDR-H2, and CDR-H3) at amino acid residues 24-34 of L1, 50-56 of L2, 89-97 of L3, H26-H35B of H1, 50-58 of H2, and 95-102 of H3. The Contact definition can include, for example, CDRs (CDR-L1, CDR-L2, CDR-L3, CDR-H1, CDR-H2, and CDR-H3) at amino acid residues 30-36 of L1, 46-55 of L2, 89-96 of L3, 30-35 of H1, 47-58 of H2, and 93-101 of H3. The Chothia definition can include, for example, CDRs (CDR-L1, CDR-L2, CDR-L3, CDR-H1, CDR-H2, and CDR-H3) at amino acid residues 24-34 of L1, 50-56 of L2, 89-97 of L3, 26-32 . . . 34 of H1, 52-56 of H2, and 95-102 of H3. With the exception of CDR1 in V_{H}, CDRs generally comprise the amino acid residues that form the hypervariable loops. The various CDRs within an antibody can be designated by their appropriate number and chain type, including, without limitation as: a) CDR-L1, CDR-L2, CDR-L3, CDR-H1, CDR-H2, and CDR-H3; b) CDRL1, CDRL2, CDRL3, CDRH1, CDRH2, and CDRH3; c) LCDR-1, LCDR-2, LCDR-3, HCDR-1, HCDR-2, and HCDR-3; or d) LCDR1, LCDR2, LCDR3, HCDR1, HCDR2, and HCDR3; etc. The term “CDR” is used herein to also encompass HVR or a “hyper variable region”, including hypervariable loops. Exemplary hypervariable loops occur at amino acid residues 26-32 (L1), 50-52 (L2), 91-96 (L3), 26-32 (H1), 53-55 (H2), and 96-101 (H3). (Chothia and Lesk, J. Mol. Biol. 196:901-917 (1987).)

The term “heavy chain variable region” as used herein refers to a region comprising at least three heavy chain CDRs. In some embodiments, the heavy chain variable region includes the three CDRs and at least FR2 and FR3. In some embodiments, the heavy chain variable region includes at least heavy chain HCDR1, framework (FR) 2, HCDR2, FR3, and HCDR3. In some embodiments, a heavy chain variable region also comprises at least a portion of an FR1 and/or at least a portion of an FR4.

The term “heavy chain constant region” as used herein refers to a region comprising at least three heavy chain constant domains, C_{H}1, C_{H}2, and C_{H}3. Of course, non-function-altering deletions and alterations within the domains are encompassed within the scope of the term “heavy chain constant region,” unless designated otherwise. Nonlimiting exemplary heavy chain constant regions include γ, δ, and α. Nonlimiting exemplary heavy chain constant regions also include ε and μ. Each heavy constant region corresponds to an antibody isotype. For example, an antibody comprising a γ constant region is an IgG antibody, an antibody comprising a δ constant region is an IgD antibody, and an antibody comprising an α constant region is an IgA antibody. Further, an antibody comprising a μ constant region is an IgM antibody, and an antibody comprising an c constant region is an IgE antibody. Certain isotypes can be further subdivided into subclasses. For example, IgG antibodies include, but are not limited to, IgG1 (comprising a γ₁ constant region), IgG2 (comprising a γ₂ constant region), IgG3 (comprising a γ₃ constant region), and IgG4 (comprising a γ₄ constant region) antibodies; IgA antibodies include, but are not limited to, IgA1 (comprising an cu constant region) and IgA2 (comprising an α₂ constant region) antibodies; and IgM antibodies include, but are not limited to, IgM1 and IgM2.

The term “heavy chain” as used herein refers to a polypeptide comprising at least a heavy chain variable region, with or without a leader sequence. In some embodiments, a heavy chain comprises at least a portion of a heavy chain constant region. The term “full-length heavy chain” as used herein refers to a polypeptide comprising a heavy chain variable region and a heavy chain constant region, with or without a leader sequence.

The term “light chain variable region” as used herein refers to a region comprising at least three light chain CDRs. In some embodiments, the light chain variable region includes the three CDRs and at least FR2 and FR3. In some embodiments, the light chain variable region includes at least light chain LCR1, framework (FR) 2, LCD2, FR3, and LCD3. For example, a light chain variable region may comprise light chain CDR1, framework (FR) 2, CDR2, FR3, and CDR3. In some embodiments, a light chain variable region also comprises at least a portion of an FR1 and/or at least a portion of an FR4.

The term “light chain constant region” as used herein refers to a region comprising a light chain constant domain, C_{L}. Nonlimiting exemplary light chain constant regions include λ, and κ. Of course, non-function-altering deletions and alterations within the domains are encompassed within the scope of the term “light chain constant region,” unless designated otherwise.

The term “light chain” as used herein refers to a polypeptide comprising at least a light chain variable region, with or without a leader sequence. In some embodiments, a light chain comprises at least a portion of a light chain constant region. The term “full-length light chain” as used herein refers to a polypeptide comprising a light chain variable region and a light chain constant region, with or without a leader sequence.

An “acceptor human framework” for the purposes herein is a framework comprising the amino acid sequence of a light chain variable domain (V_{L}) framework or a heavy chain variable domain (V_{H}) framework derived from a human immunoglobulin framework or a human consensus framework, as defined below. An acceptor human framework derived from a human immunoglobulin framework or a human consensus framework can comprise the same amino acid sequence thereof, or it can contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the V_{L}, acceptor human framework is identical in sequence to the V_{L} human immunoglobulin framework sequence or human consensus framework sequence.

“Affinity” refers to the strength of the sum total of noncovalent interactions between a single binding site of a molecule (for example, an antibody) and its binding partner (for example, an antigen). The affinity of a molecule X for its partner Y can generally be represented by the dissociation constant (K_{D}). Affinity can be measured by common methods known in the art (such as, for example, ELISA K_{D}, KinExA, bio-layer interferometry (BLI), and/or surface plasmon resonance devices (such as a BIAcore® device), including those described herein).

The term “K_{D}”, as used herein, refers to the equilibrium dissociation constant of an antibody-antigen interaction.

In some embodiments, the “K_{D},” “K_{d},” “Kd” or “Kd value” of the antibody is measured by using surface plasmon resonance assays using a BIACORE®-2000 or a BIACORE®-3000 (BIAcore, Inc., Piscataway, N.J.) at 25° C. with immobilized antigen CM5 chips at ˜10 response units (RU). Briefly, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μL/minute to achieve approximately 10 response units (RU) of coupled protein. Following the injection of antigen, 1 M ethanolamine is injected to block unreacted groups. For kinetics measurements, serial dilutions of polypeptide, for example, full length antibody, are injected in PBS with 0.05% TWEEN-20™ surfactant (PBST) at 25° C. at a flow rate of approximately 25 μL/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (K_{d}) is calculated as the ratio k_{off}/kₒₙ. See, for example, Chen et al., J. Mol. Biol. 293:865-881 (1999). If the on-rate exceeds 10⁶ M⁻¹s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

In some embodiments, the difference between said two values (for example, Ct values) is substantially the same, for example, less than about 50%, less than about 40%, less than about 30%, less than about 20%, and/or less than about 10% as a function of the reference/comparator value.

In some embodiments, the difference between said two values (for example, Ct values) is substantially different, for example, greater than about 10%, greater than about 20%, greater than about 30%, greater than about 40%, and/or greater than about 50% as a function of the value for the reference/comparator molecule.

“Surface plasmon resonance” denotes an optical phenomenon that allows for the analysis of real-time biospecific interactions by detection of alterations in protein concentrations within a biosensor matrix, for example using the BIAcore′ system (BIAcore International AB, a GE Healthcare company, Uppsala, Sweden and Piscataway, N.J.). For further descriptions, see Jonsson et al. (1993) Ann. Biol. Clin. 51:19-26.

“Biolayer interferometry” refers to an optical analytical technique that analyzes the interference pattern of light reflected from a layer of immobilized protein on a biosensor tip and an internal reference layer. Changes in the number of molecules bound to the biosensor tip cause shifts in the interference pattern that can be measured in real-time. A nonlimiting exemplary device for biolayer interferometry is ForteBio Octet® RED96 system (Pall Corporation). See, e.g., Abdiche et al., 2008, Anal. Biochem. 377: 209-277.

The term “kₒₙ”, as used herein, refers to the rate constant for association of an antibody to an antigen. Specifically, the rate constants (kₒₙ and k_{off}) and equilibrium dissociation constants are measured using IgGs (bivalent) with monovalent ICOS antigen. “Kₒₙ”, “kₒₙ”, “association rate constant”, or “ka”, are used interchangeably herein. The value indicates the binding rate of a binding protein to its target antigen or the rate of complex formation between an antibody and antigen, shown by the equation:

Antibody(“Ab”)+Antigen(“Ag”)→Ab-Ag.

The term “k_{off}”, as used herein, refers to the rate constant for dissociation of an antibody from the antibody/antigen complex. k_{off} is also denoted as “K_{off}” or the “dissociation rate constant”. This value indicates the dissociation rate of an antibody from its target antigen or separation of Ab-Ag complex over time into free antibody and antigen as shown by the equation:

Ab+Ag←Ab-Ag

The term “biological activity” refers to any one or more biological properties of a molecule (whether present naturally as found in vivo, or provided or enabled by recombinant means). Biological properties include, but are not limited to, binding a receptor, inducing cell proliferation, inhibiting cell growth, inducing other cytokines, inducing apoptosis, and enzymatic activity. In some embodiments, biological activity of an ICOS protein includes, for example, costimulation of T cell proliferation and cytokine secretion associated with Th1 and Th2 cells; modulation of Treg cells; effects on T cell differentiation including modulation of transcription factor gene expression; induction of signaling through PI3K and AKT pathways; and mediating ADCC.

An “affinity matured” antibody refers to an antibody with one or more alterations in one or more CDRs compared to a parent antibody which does not possess such alterations, such alterations resulting in an improvement in the affinity of the antibody for antigen.

A “chimeric antibody” as used herein refers to an antibody in which a portion of the heavy and/or light chain is derived from a particular source or species, while at least a part of the remainder of the heavy and/or light chain is derived from a different source or species. In some embodiments, a chimeric antibody refers to an antibody comprising at least one variable region from a first species (such as mouse, rat, cynomolgus monkey, etc.) and at least one constant region from a second species (such as human, cynomolgus monkey, etc.). In some embodiments, a chimeric antibody comprises at least one mouse variable region and at least one human constant region. In some embodiments, a chimeric antibody comprises at least one cynomolgus variable region and at least one human constant region. In some embodiments, all of the variable regions of a chimeric antibody are from a first species and all of the constant regions of the chimeric antibody are from a second species. The chimeric construct can also be a functional fragment, as noted above.

A “humanized antibody” as used herein refers to an antibody in which at least one amino acid in a framework region of a non-human variable region has been replaced with the corresponding amino acid from a human variable region. In some embodiments, a humanized antibody comprises at least one human constant region or fragment thereof. In some embodiments, a humanized antibody is an antibody fragment, such as Fab, an scFv, a (Fab′)₂, etc. The term humanized also denotes forms of non-human (for example, murine) antibodies that are chimeric immunoglobulins, immunoglobulin chains, or fragments thereof (such as Fv, Fab, Fab′, F(ab′)2 or other antigen-binding subsequences of antibodies) that contain minimal sequence of non-human immunoglobulin. Humanized antibodies can include human immunoglobulins (recipient antibody) in which residues from a complementary determining region (CDR) of the recipient are substituted by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody can comprise residues that are found neither in the recipient antibody nor in the imported CDR or framework sequences, but are included to further refine and optimize antibody performance. In general, the humanized antibody can comprise substantially all of at least one, and typically two, variable domains, in which all or substantially all of the CDR regions correspond to those of a non-human immunoglobulin and all or substantially all of the FR regions are those of a human immunoglobulin consensus sequence. In some embodiments, the humanized antibody can also comprise at least a portion of an immunoglobulin constant region or domain (Fc), typically that of a human immunoglobulin. Other forms of humanized antibodies have one or more CDRs (CDR L1, CDR L2, CDR L3, CDR H1, CDR H2, and/or CDR H3) which are altered with respect to the original antibody, which are also termed one or more CDRs “derived from” one or more CDRs from the original antibody. As will be appreciated, a humanized sequence can be identified by its primary sequence and does not necessarily denote the process by which the antibody was created.

A “CDR-grafted antibody” as used herein refers to a humanized antibody in which one or more complementarity determining regions (CDRs) of a first (non-human) species have been grafted onto the framework regions (FRs) of a second (human) species.

A “human antibody” as used herein encompasses antibodies produced in humans, antibodies produced in non-human animals that comprise human immunoglobulin genes, such as XenoMouse® mice, and antibodies selected using in vitro methods, such as phage display (Vaughan et al., 1996, Nature Biotechnology, 14:309-314; Sheets et al., 1998, Proc. Natl. Acad. Sci. (USA) 95:6157-6162; Hoogenboom and Winter, 1991, J. Mol. Biol., 227:381; Marks et al., 1991, J. Mol. Biol., 222:581), wherein the antibody repertoire is based on a human immunoglobulin sequence. The term “human antibody” denotes the genus of sequences that are human sequences. Thus, the term is not designating the process by which the antibody was created, but the genus of sequences that are relevant.

A “functional Fc region” possesses an “effector function” of a native sequence Fc region. Exemplary “effector functions” include Fc receptor binding; Clq binding; CDC; ADCC; phagocytosis; down regulation of cell surface receptors (for example B cell receptor; BCR), etc. Such effector functions generally require the Fc region to be combined with a binding domain (for example, an antibody variable domain) and can be assessed using various assays.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification. In some embodiments, a “variant Fc region” comprises an amino acid sequence which differs from that of a native sequence Fc region by virtue of at least one amino acid modification, yet retains at least one effector function of the native sequence Fc region. In some embodiments, the variant Fc region has at least one amino acid substitution compared to a native sequence Fc region or to the Fc region of a parent polypeptide, for example, from about one to about ten amino acid substitutions, and preferably, from about one to about five amino acid substitutions in a native sequence Fc region or in the Fc region of the parent polypeptide. In some embodiments, the variant Fc region herein will possess at least about 80% sequence identity with a native sequence Fc region and/or with an Fc region of a parent polypeptide, at least about 90% sequence identity therewith, at least about 95%, at least about 96%, at least about 97%, at least about 98%, or at least about 99% sequence identity therewith.

“Fe receptor” or “FcR” describes a receptor that binds to the Fc region of an antibody. In some embodiments, an FcγR is a native human FcR. In some embodiments, an FcR is one which binds an IgG antibody (a gamma receptor) and includes receptors of the FcγRI, FcγRII, and FcγRIII subclasses, including allelic variants and alternatively spliced forms of those receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain Inhibiting receptor FcγRIIB contains an immunoreceptor tyrosine-based inhibition motif (ITIM) in its cytoplasmic domain. (see, for example, Daeron, Annu. Rev. Immunol. 15:203-234 (1997)). FcRs are reviewed, for example, in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein.

The term “Fe receptor” or “FcR” also includes the neonatal receptor, FcRn, which is responsible for the transfer of maternal IgGs to the fetus (Guyer et al., J. Immunol. 117:587 (1976) and Kim et al., J. Immunol. 24:249 (1994)) and regulation of homeostasis of immunoglobulins. Methods of measuring binding to FcRn are known (see, for example, Ghetie and Ward., Immunol. Today 18(12):592-598 (1997); Ghetie et al., Nature Biotechnology, 15(7):637-640 (1997); Hinton et al., J. Biol. Chem. 279(8):6213-6216 (2004); WO 2004/92219 (Hinton et al.).

“Effector functions” refer to biological activities attributable to the Fc region of an antibody, which vary with the antibody isotype. Examples of antibody effector functions include: Clq binding and complement dependent cytotoxicity (CDC); Fc receptor binding; antibody-dependent cell-mediated cytotoxicity (ADCC); phagocytosis; down regulation of cell surface receptors (for example B cell receptor); and B cell activation.

“Human effector cells” are leukocytes which express one or more FcRs and perform effector functions. In some embodiments, the cells express at least FcγRIII and perform ADCC effector function(s). Examples of human leukocytes which mediate ADCC include peripheral blood mononuclear cells (PBMC), natural killer (NK) cells, monocytes, cytotoxic T cells, and neutrophils. The effector cells may be isolated from a native source, for example, from blood.

“Antibody-dependent cell-mediated cytotoxicity” or “ADCC” refers to a form of cytotoxicity in which secreted Ig bound onto Fc receptors (FcRs) present on certain cytotoxic cells (for example NK cells, neutrophils, and macrophages) enable these cytotoxic effector cells to bind specifically to an antigen-bearing target cell and subsequently kill the target cell with cytotoxins. The primary cells for mediating ADCC, NK cells, express FcγRIII only, whereas monocytes express FcγRI, FcγRII, and FcγRIII FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in US Pat. No. 5,500,362 or U.S. Pat. No. 5,821,337 or U.S. Pat. No. 6,737,056 (Presta), may be performed. Useful effector cells for such assays include PBMC and NK cells. Alternatively, or additionally, ADCC activity of the molecule of interest may be assessed in vivo, for example, in an animal model such as that disclosed in Clynes et al. Proc. Natl. Acad. Sci. (USA) 95:652-656 (1998). Additional polypeptide variants with altered Fc region amino acid sequences (polypeptides with a variant Fc region) and increased or decreased ADCC activity are described, for example, in U.S. Pat. Nos. 7,923,538, and 7,994,290.

“Complement dependent cytotoxicity” or “CDC” refers to the lysis of a target cell in the presence of complement. Activation of the classical complement pathway is initiated by the binding of the first component of the complement system (Clq) to antibodies (of the appropriate subclass), which are bound to their cognate antigen. To assess complement activation, a CDC assay, for example, as described in Gazzano-Santoro et al., J. Immunol. Methods 202:163 (1996), may be performed. Polypeptide variants with altered Fc region amino acid sequences (polypeptides with a variant Fc region) and increased or decreased Clq binding capability are described, for example, in U.S. Pat. No. 6,194,551 B1, U.S. Pat. Nos. 7,923,538, 7,994,290 and WO 1999/51642. See also, for example, Idusogie et al., J. Immunol. 164: 4178-4184 (2000).

A polypeptide variant with “altered” FcR binding affinity or ADCC activity is one which has either enhanced or diminished FcR binding activity and/or ADCC activity compared to a parent polypeptide or to a polypeptide comprising a native sequence Fc region. The polypeptide variant which “displays increased binding” to an FcR binds at least one FcR with better affinity than the parent polypeptide. The polypeptide variant which “displays decreased binding” to an FcR, binds at least one FcR with lower affinity than a parent polypeptide. Such variants which display decreased binding to an FcR may possess little or no appreciable binding to an FcR, for example, 0-20% binding to the FcR compared to a native sequence IgG Fc region.

The polypeptide variant which “mediates antibody-dependent cell-mediated cytotoxicity (ADCC) in the presence of human effector cells more effectively” than a parent antibody is one which in vitro or in vivo is more effective at mediating ADCC, when the amounts of polypeptide variant and parent antibody used in the assay are essentially the same. Generally, such variants will be identified using the in vitro ADCC assay as herein disclosed, but other assays or methods for determining ADCC activity, for example in an animal model etc., are contemplated.

The term “substantially similar” or “substantially the same,” as used herein, denotes a sufficiently high degree of similarity between two or more numeric values such that one of skill in the art would consider the difference between the two or more values to be of little or no biological and/or statistical significance within the context of the biological characteristic measured by said value. In some embodiments the two or more substantially similar values differ by no more than about any one of 5%, 10%, 15%, 20%, 25%, or 50%.

The phrase “substantially different,” as used herein, denotes a sufficiently high degree of difference between two numeric values such that one of skill in the art would consider the difference between the two values to be of statistical significance within the context of the biological characteristic measured by said values. In some embodiments, the two substantially different numeric values differ by greater than about any one of 10%, 20%, 25%, 30%, 35%, 40%, 45%, 50%, 60%, 70%, 80%, 90%, or 100%.

The phrase “substantially reduced,” as used herein, denotes a sufficiently high degree of reduction between a numeric value and a reference numeric value such that one of skill in the art would consider the difference between the two values to be of statistical significance within the context of the biological characteristic measured by said values. In some embodiments, the substantially reduced numeric values is reduced by greater than about any one of 10%, 20%, 25%, 30%, 35%, 40%, 45%, 50%, 60%, 70%, 80%, 90%, or 100% compared to the reference value.

The term “leader sequence” refers to a sequence of amino acid residues located at the N-terminus of a polypeptide that facilitates secretion of a polypeptide from a mammalian cell. A leader sequence can be cleaved upon export of the polypeptide from the mammalian cell, forming a mature protein. Leader sequences can be natural or synthetic, and they can be heterologous or homologous to the protein to which they are attached.

A “native sequence” polypeptide comprises a polypeptide having the same amino acid sequence as a polypeptide found in nature. Thus, a native sequence polypeptide can have the amino acid sequence of naturally occurring polypeptide from any mammal. Such native sequence polypeptide can be isolated from nature or can be produced by recombinant or synthetic means. The term “native sequence” polypeptide specifically encompasses naturally occurring truncated or secreted forms of the polypeptide (for example, an extracellular domain sequence), naturally occurring variant forms (for example, alternatively spliced forms) and naturally occurring allelic variants of the polypeptide.

A polypeptide “variant” means a biologically active polypeptide having at least about 80% amino acid sequence identity with the native sequence polypeptide after aligning the sequences and introducing gaps, if necessary, to achieve the maximum percent sequence identity, and not considering any conservative substitutions as part of the sequence identity. Such variants include, for instance, polypeptides wherein one or more amino acid residues are added, or deleted, at the N- or C-terminus of the polypeptide. In some embodiments, a variant will have at least about 80% amino acid sequence identity. In some embodiments, a variant will have at least about 90% amino acid sequence identity. In some embodiments, a variant will have at least about 95% amino acid sequence identity with the native sequence polypeptide.

As used herein, “Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

An amino acid substitution may include but are not limited to the replacement of one amino acid in a polypeptide with another amino acid. Exemplary substitutions are shown in Table 1. Amino acid substitutions may be introduced into an antibody of interest and the products screened for a desired activity, for example, retained/improved antigen binding, decreased immunogenicity, or improved ADCC or CDC.

TABLE 1

| Original Residue | Exemplary Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

The term “vector” is used to describe a polynucleotide that can be engineered to contain a cloned polynucleotide or polynucleotides that can be propagated in a host cell. A vector can include one or more of the following elements: an origin of replication, one or more regulatory sequences (such as, for example, promoters and/or enhancers) that regulate the expression of the polypeptide of interest, and/or one or more selectable marker genes (such as, for example, antibiotic resistance genes and genes that can be used in colorimetric assays, for example, (3-galactosidase). The term “expression vector” refers to a vector that is used to express a polypeptide of interest in a host cell.

A “host cell” refers to a cell that may be or has been a recipient of a vector or isolated polynucleotide. Host cells may be prokaryotic cells or eukaryotic cells. Exemplary eukaryotic cells include mammalian cells, such as primate or non-primate animal cells; fungal cells, such as yeast; plant cells; and insect cells. Nonlimiting exemplary mammalian cells include, but are not limited to, NSO cells, PER.C6° cells (Crucell), and 293 and CHO cells, and their derivatives, such as 293-6E and DG44 cells, respectively. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) a provided herein.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, for example, in the case of an RNA polynucleotide. Thus, a DNA polynucleotide that is contained in a vector inside a host cell may be referred to as “isolated”.

The terms “individual” or “subject” are used interchangeably herein to refer to an animal; for example a mammal. In some embodiments, methods of treating mammals, including, but not limited to, humans, rodents, simians, felines, canines, equines, bovines, porcines, ovines, caprines, mammalian laboratory animals, mammalian farm animals, mammalian sport animals, and mammalian pets, are provided. In some examples, an “individual” or “subject” refers to an individual or subject in need of treatment for a disease or disorder. In some embodiments, the subject to receive the treatment can be a patient, designating the fact that the subject has been identified as having a disorder of relevance to the treatment, or being at adequate risk of contracting the disorder.

The term “sample” or “patient sample” as used herein, refers to a composition that is obtained or derived from a subject of interest that contains a cellular and/or other molecular entity that is to be characterized and/or identified, for example based on physical, biochemical, chemical and/or physiological characteristics. For example, the phrase “disease sample” and variations thereof refers to any sample obtained from a subject of interest that would be expected or is known to contain the cellular and/or molecular entity that is to be characterized. By “tissue or cell sample” is meant a collection of similar cells obtained from a tissue of a subject or patient. The source of the tissue or cell sample may be solid tissue as from a fresh, frozen and/or preserved organ or tissue sample or biopsy or aspirate; blood or any blood constituents; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. The tissue sample may contain compounds which are not naturally intermixed with the tissue in nature such as preservatives, anticoagulants, buffers, fixatives, nutrients, antibiotics, or the like.

A “reference sample”, “reference cell”, or “reference tissue”, as used herein, refers to a sample, cell or tissue obtained from a source known, or believed, not to be afflicted with the disease or condition for which a method or composition of the invention is being used to identify. In some embodiments, a reference sample, reference cell or reference tissue is obtained from a healthy part of the body of the same subject or patient in whom a disease or condition is being identified using a composition or method of the invention. In some embodiments, a reference sample, reference cell or reference tissue is obtained from a healthy part of the body of one or more individuals who are not the subject or patient in whom a disease or condition is being identified using a composition or method of the invention.

A “disease” or “disorder” as used herein refers to a condition where treatment is needed and/or desired.

“Cancer” and “tumor,” as used herein, are interchangeable terms that refer to any abnormal cell or tissue growth or proliferation in an animal. As used herein, the terms “cancer” and “tumor” encompass solid and hematological/lymphatic cancers and also encompass malignant, pre-malignant, and benign growth, such as dysplasia. Examples of cancer include but are not limited to, carcinoma, lymphoma, blastoma, sarcoma, and leukemia. More particular non-limiting examples of such cancers include squamous cell cancer, small-cell lung cancer, pituitary cancer, esophageal cancer, astrocytoma, soft tissue sarcoma, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma (including uterine corpus endometrial carcinoma), salivary gland carcinoma, kidney cancer, renal cancer, liver cancer, prostate cancer, vulval cancer, thyroid cancer, hepatic carcinoma, brain cancer, testis cancer, cholangiocarcinoma, gallbladder carcinoma, gastric cancer, melanoma, mesothelioma, and various types of head and neck cancer.

As used herein, “treatment” is an approach for obtaining beneficial or desired clinical results. “Treatment” as used herein, covers any administration or application of a therapeutic for disease in a mammal, including a human. For purposes of this disclosure, beneficial or desired clinical results include, but are not limited to, any one or more of: alleviation of one or more symptoms, diminishment of extent of disease, preventing or delaying spread (for example, metastasis, for example metastasis to the lung or to the lymph node) of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total). Also encompassed by “treatment” is a reduction of pathological consequence of a proliferative disease. The methods provided herein contemplate any one or more of these aspects of treatment. In-line with the above, the term treatment does not require one-hundred percent removal of all aspects of the disorder.

“Ameliorating” means a lessening or improvement of one or more symptoms as compared to not administering an anti-ICOS antibody. “Ameliorating” also includes shortening or reduction in duration of a symptom.

In the context of cancer, the term “treating” includes any or all of: inhibiting growth of cancer cells, inhibiting replication of cancer cells, lessening of overall tumor burden and ameliorating one or more symptoms associated with the disease.

The term “biological sample” means a quantity of a substance from a living thing or formerly living thing. Such substances include, but are not limited to, blood, (for example, whole blood), plasma, serum, urine, amniotic fluid, synovial fluid, endothelial cells, leukocytes, monocytes, other cells, organs, tissues, bone marrow, lymph nodes and spleen.

A sample that has an “elevated level of ICOS” or “expresses ICOS at an elevated level” or is “ICOS^{HIGH}” means, in some embodiments, that the level of ICOS that is such that one of skill in the art would conclude that the cancer may be treatable with an anti-ICOS agonist therapy, such as an antibody provided herein. In some embodiments, an “elevated level of ICOS” is one in which 1% of the cells within a tumor sample show staining for ICOS. In some embodiments a “high level” in regard to ICOS is 1% or more staining, for example, 1, 5, 10, 15, 20, 30, 40, 50, 60, 70, 80, 90, or 100% of the cells within the tumor sample show staining. In some embodiments, the ICOS levels can be measured by chromogenic IHC or immunofluorescence IHC (Aqua scoring).

A sample that “expresses ICOS” or has “positive staining for ICOS” or is “ICOS positive” means, in some embodiments, that 1% or more of the cells in a sample show staining for ICOS. In some embodiments, a sample that is ICOS positive displays at least weak, moderate, and/or strong cell staining (based on membrane expression of ICOS). A sample with moderate or strong cell staining for ICOS is also considered to be “ICOS^{HIGH}.”

A sample that has a “low level of PD-L1” or expresses “PD-L1 at a low level” or is “PD-L1^{LOw}” means that the level of PD-L1 is below the threshold level of expression for a cancer that is normally indicated for treatment with a PD-1 therapy. In some embodiments, a “low level of PD-L1” is one in which less than 5% of the cells in the tumor show membrane staining for PD-L1. In some embodiments a “low level” in regard to PD-L1 is less than 5% staining, for example, 4%, 3%, 2%, 1%, or 0% of the cells of the tumor show staining. In some embodiments, the PD-L1 levels can be measured by chromogenic IHC or immunofluorescence IHC (Aqua scoring). A sample that expresses no detectable PD-L1 can also be said to “express a low level of PD-L1.” Thus, no detectable PD-L1 is encompassed within the term “low.”

A sample that has an “elevated level of PD-L1” or “expresses PD-L1 at an elevated level” or is “PD-L1^{HIGH}” means that the level of PD-L1 that is such that one of skill in the art would conclude that the cancer may be treatable with a PD-1 therapy. In some embodiments, an “elevated level of PD-L1” is one in which 1% of the cells in the tumor or more have membrane staining of PD-L1. In some embodiments a “high level” in regard to PD-L1 is 5% or more staining, for example, 5, 10, 20, 30, 40, 50, 60, 70, 80, 90, or 100% of the cells of the tumor show staining. In some embodiments, the PD-L1 levels can be measured by chromogenic IHC or immunofluorescence IHC (Aqua scoring).

A sample that “expresses PD-L1” or has “positive staining for PD-L1” or is “PD-L1 positive” means that 1% or more of the cells have membrane staining for PD-L1. In some embodiments, a sample that is PD-L1 positive displays at least weak, moderate, and/or strong cell staining (based on membrane expression of PD-L1). A sample with moderate or strong cell staining for PD-L1 is also considered to be “PD-L1^{HIGH}.”

A sample that “lacks PD-L1 expression” or has “negative staining for PD-L1” or is “PD-L1 negative” means that PD-L1 expression on the surface of cells of the sample is undetectable by IHC, such as chromogenic IHC or immunofluorescence IHC (Aqua scoring). A PD-L1 negative sample is also be considered to be “PD-L1^{LOW}.”

In some embodiments, any method for measuring the level of PD-L1 can be employed. In some embodiments, this can include using the PD-L1 IHC 22C3 pharmDx test (Dako Inc., Carpinteria, Calif.), which is a clinically validated and FDA approved test for evaluation of PD-L1 expression in NSCLC. PD-L1 IHC 22C3 pharmDx is a qualitative immunohistochemical assay using monoclonal mouse anti-PD-L1 antibody, (clone 22C3), that can be used in the detection of PD-L1 protein in formalin-fixed paraffin-embedded (FFPE) Non-Small Cell Lung Cancer (NSCLC) tissues. The assay can be performed on Autostainer Link 48 system and visualized using the EnVision FLEX system. PD-L1 protein expression is qualified using Tumor Proportion Score (TPS), which is the percentage of viable tumor cells showing partial or complete membrane staining. In some embodiments, the specimen is considered PD-L1 positive if TPS≥50% of the viable tumor cells exhibit membrane staining at any intensity. PD-L1 IHC 22C3 pharmDx is indicated as an aid in identifying NSCLC patients for treatment with KEYTRUDA® (pembrolizumab). Additional details on the scoring system and response to pembrolizumab are described in the article by Garon et al. (N Engl J Med 2015; 372:2018-28). In some embodiments, NSCLC patient specimens can be considered positive for PD-L1 expression if Tumor Proportion Score is ≥50% of the of viable tumor cells exhibit membrane staining (partial or complete) at any intensity (i.e. ≥1+). In some embodiments, this can be in specific regard to antibody clone 22C3. In some embodiments, if TPS=5% to 50% of the viable tumor cells exhibit membrane staining at any intensity, the sample and/or patient is considered to be PD-L1 positive. In some embodiments, if TPS≥50% of the viable tumor cells exhibit membrane staining at any intensity, the sample and/or patient is considered to be PD-L1^{HIGH}.

The terms “microsatellite instability high” and “MSI-high” refer to cancer comprising genetic instability (e.g., an expansion or reduction in the length of the microsatellites) in 2 or more of the 5 markers (loci): BAT25, BAT26, D5S346, D2S123, and D17S250, as determined by PCR analysis. See, e.g., Boland et al., 1998, Cancer Res. 58: 5248-5257.

The terms “microsatellite instability low” and “MSI-low” refer to cancer comprising genetic instability (e.g., an expansion or reduction in the length of the microsatellites) in 1 of the 5 markers (loci): BAT25, BAT26, D5S346, D2S123, and D17S250, as determined by PCR analysis. See, e.g., Boland et al., 1998, Cancer Res. 58: 5248-5257.

The terms “microsatellite instability positive” and “MSI-positive” refer to tumors that are MSI-high or MSI-low. A cancer is also considered to be MSI-positive if one or more mismatch repair proteins selected from MLH1, MSH2, PMS2, and MSH6 are absent by immunohistochemistry (IHC).

The terms “microsatellite stable” and “MSS” refer to cancer comprising genetic instability (e.g., an expansion or reduction in the length of the microsatellites) in none of the 5 markers (loci): BAT25, BAT26, D5S346, D2S123, and D17S250, as determined by PCR analysis. See, e.g., Boland et al., 1998, Cancer Res. 58: 5248-5257.

The term “control” refers to a composition known to not contain an analyte (“negative control”) or to contain analyte (“positive control”). A positive control can comprise a known concentration of analyte. “Control,” “positive control,” and “calibrator” may be used interchangeably herein to refer to a composition comprising a known concentration of analyte. A “positive control” can be used to establish assay performance characteristics and is a useful indicator of the integrity of reagents (for example, analytes).

“Predetermined cutoff” and “predetermined level” refer generally to an assay cutoff value that is used to assess diagnostic/prognostic/therapeutic efficacy results by comparing the assay results against the predetermined cutoff/level, where the predetermined cutoff/level already has been linked or associated with various clinical parameters (for example, severity of disease, progression/nonprogression/improvement, etc.). While the present disclosure may provide exemplary predetermined levels, it is well-known that cutoff values may vary depending on the nature of the immunoassay (for example, antibodies employed, etc.). It further is well within the skill of one of ordinary skill in the art to adapt the disclosure herein for other immunoassays to obtain immunoassay-specific cutoff values for those other immunoassays based on this disclosure. Whereas the precise value of the predetermined cutoff/level may vary between assays, correlations as described herein (if any) may be generally applicable.

The terms “inhibition” or “inhibit” refer to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to a reference. In some embodiments, by “reduce” or “inhibit” is meant the ability to cause an overall decrease of 20% or greater. In some embodiments, by “reduce” or “inhibit” is meant the ability to cause an overall decrease of 50% or greater. In some embodiments, by “reduce” or “inhibit” is meant the ability to cause an overall decrease of 75%, 85%, 90%, 95%, or greater. In some embodiments, the amount noted above is inhibited or decreased over a period of time, relative to a control dose (such as a placebo) over the same period of time. A “reference” as used herein, refers to any sample, standard, or level that is used for comparison purposes. A reference may be obtained from a healthy and/or non-diseased sample. In some examples, a reference may be obtained from an untreated sample. In some examples, a reference is obtained from a non-diseased on non-treated sample of a subject individual. In some examples, a reference is obtained from one or more healthy individuals who are not the subject or patient.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease. Unless otherwise specified, the terms “reduce”, “inhibit”, or “prevent” do not denote or require complete prevention over all time.

As used herein, to “suppress” a function or activity is to reduce the function or activity when compared to otherwise same conditions except for a condition or parameter of interest, or alternatively, as compared to another condition. For example, an antibody which suppresses tumor growth reduces the rate of growth of the tumor compared to the rate of growth of the tumor in the absence of the antibody.

A “therapeutically effective amount” of a substance/molecule, agonist or antagonist may vary according to factors such as the disease state, age, sex, and weight of the individual, and the ability of the substance/molecule, agonist or antagonist to elicit a desired response in the individual. A therapeutically effective amount is also one in which any toxic or detrimental effects of the substance/molecule, agonist or antagonist are outweighed by the therapeutically beneficial effects. A therapeutically effective amount may be delivered in one or more administrations. A therapeutically effective amount refers to an amount effective, at doses and for periods of time necessary, to achieve the desired therapeutic and/or prophylactic result.

A “prophylactically effective amount” refers to an amount effective, at doses and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

The terms “pharmaceutical formulation” and “pharmaceutical composition” refer to a preparation which is in such form as to permit the biological activity of the active ingredient(s) to be effective, and which contains no additional components which are unacceptably toxic to a subject to which the formulation would be administered. Such formulations may be sterile.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the doses and concentrations employed and is compatible with other ingredients of the formulation. The pharmaceutically acceptable carrier is appropriate for the formulation employed.

A “sterile” formulation is aseptic or essentially free from living microorganisms and their spores.

A “PD-1 therapy” encompasses any therapy that modulates PD-1 binding to PD-L1 and/or PD-L2. PD-1 therapies may, for example, directly interact with PD-1 and/or PD-L1. In some embodiments, a PD-1 therapy includes a molecule that directly binds to and/or influences the activity of PD-1. In some embodiments, a PD-1 therapy includes a molecule that directly binds to and/or influences the activity of PD-L1. Thus, an antibody that binds to PD-1 or PD-L1 and blocks the interaction of PD-1 to PD-L1 is a PD-1 therapeutic. When a desired subtype of PD-1 therapy is intended, it will be designated by the phrase “PD-1 specific” for a therapy involving a molecule that interacts directly with PD-1, or “PD-L1 specific” for a molecule that interacts directly with PD-L1, as appropriate. Unless designated otherwise, all disclosure contained herein regarding PD-1 therapy applies to PD-1 therapy generally, as well as PD-1 specific and/or PD-L1 specific therapies. Nonlimiting exemplary PD-1 therapies include nivolumab (anti-PD-1 antibody; BMS-936558, MDX-1106, ONO-4538; OPDIVO®; Bristol-Myers Squibb); pidilizumab (anti-PD-1 antibody, CureTech), pembrolizumab (anti-PD-1 antibody; KEYTRUIDA®, MK-3475, lambrolizumab); durvalumab (anti-PD-L1 antibody, MEDI-4736; AstraZeneca/MedImmune); RG-7446; MSB-0010718C; AMP-224; BMS-936559 (an anti-PD-L1 antibody; Bristol-Myers Squibb); AMP-514; MDX-1105; ANB-011; anti-LAG-3/PD-1; anti-PD-1 Ab (CoStim); anti-PD-1 Ab (Kadmon Pharm.); anti-PD-1 Ab (Immunovo); anti-TIM-3/PD-1 Ab (AnaptysBio); anti-PD-L1 Ab (CoStim/Novartis); atezolizumab (an anti-PD-L1 antibody, Genentech/Roche); avelumab (an anti-PD-L1 antibody, MSB0010718C, Pfizer); KD-033, PD-1 antagonist (Agenus); STI-A1010; STI-A1110; TSR-042; and other antibodies that are directed against programmed death-1 (PD-1) or programmed death ligand 1 (PD-L1).

The term “IDO inhibitor” refers to an agent capable of inhibiting the activity of indoleamine 2,3-dioxygenase (IDO) and thereby reversing IDO-mediated immunosuppression. The IDO inhibitor may inhibit IDO1 and/or IDO2 (INDOL1). An IDO inhibitor may be a reversible or irreversible IDO inhibitor. A “reversible IDO inhibitor” is a compound that reversibly inhibits IDO enzyme activity either at the catalytic site or at a non-catalytic site and an “irreversible IDO inhibitor” is a compound that irreversibly inhibits IDO enzyme activity by forming a covalent bond with the enzyme. Nonlimiting exemplary IDO inhibitors include Indoximod (New Link Genetics), epicadostat (Incyte Corp.), 1-methyl-D-tryptophan (New Link Genetics), and GDC-0919 (Genentech).

A “chimeric antigen receptor T cell therapy” or “CAR-T therapy” refers to a therapeutic agent comprising a T cell genetically modified to express a receptor that recognizes an antigen expressed by tumor cell. The antigen may be an antigen specifically expressed by the tumor or an antigen expressed by both cancerous cells and healthy tissue. In some embodiments CAR-T therapy is adoptive CAR-T therapy, in which a patients T cells are removed and modified to express the chimeric antigen receptor, and then returned to the patient. See, e.g., Dai et al., 2016, J Natl Cancer Inst, 108 (7): djv439, doi: 10.1093/jnci/djv439; Gill et al., 2015, Blood Rev, pii: 50268-960X(15)00080-6, doi: 10.1016/j.blre.2015.10.003; Gill et al., 2015, Immunol Rev, 263(1):68-89. doi: 10.1111/imr.12243.

Administration “in combination with” one or more further therapeutic agents includes simultaneous (concurrent) and consecutive or sequential administration in any order.

The term “concurrently” is used herein to refer to administration of two or more therapeutic agents, where at least part of the administration overlaps in time or where the administration of one therapeutic agent falls within a short period of time relative to administration of the other therapeutic agent. For example, the two or more therapeutic agents are administered with a time separation of no more than about a specified number of minutes.

The term “sequentially” is used herein to refer to administration of two or more therapeutic agents where the administration of one or more agent(s) continues after discontinuing the administration of one or more other agent(s), or wherein administration of one or more agent(s) begins before the administration of one or more other agent(s). For example, administration of the two or more therapeutic agents are administered with a time separation of more than about a specified number of minutes.

As used herein, “in conjunction with” refers to administration of one treatment modality in addition to another treatment modality. As such, “in conjunction with” refers to administration of one treatment modality before, during or after administration of the other treatment modality to the individual.

The term “package insert” is used to refer to instructions customarily included in commercial packages of therapeutic products, that contain information about the indications, usage, dose, administration, combination therapy, contraindications and/or warnings concerning the use of such therapeutic products. These are also referred to as the Full Prescribing Information for a product in the U.S.

An “article of manufacture” is any manufacture (for example, a package or container) or kit comprising at least one reagent, for example, a medicament for treatment of a disease or disorder (for example, cancer), or a probe for specifically detecting a biomarker described herein. In some embodiments, the manufacture or kit is promoted, distributed, or sold as a unit for performing the methods described herein.

The terms “label” and “detectable label” mean a moiety attached to a polynucleotide or polypeptide to render a reaction (for example, polynucleotide amplification or antibody binding) detectable. The polynucleotide or polypeptide comprising the label may be referred to as “detectably labeled.” Thus, the term “labeled binding protein” refers to a protein with a label incorporated that provides for the identification of the binding protein. The term “labeled oligonucleotide,” “labeled primer,” “labeled probe,” etc. refers to a polynucleotide with a label incorporated that provides for the identification of nucleic acids that comprise or are hybridized to the labeled oligonucleotide, primer, or probe. In some embodiments, the label is a detectable marker that can produce a signal that is detectable by visual or instrumental means, for example, incorporation of a radiolabeled amino acid or attachment to a polypeptide of biotinyl moieties that can be detected by marked avidin (for example, streptavidin containing a fluorescent marker or enzymatic activity that can be detected by optical or colorimetric methods). Examples of labels include, but are not limited to, the following: radioisotopes or radionuclides (for example, ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, or ¹⁵³Sm); chromogens, fluorescent labels (for example, FITC, rhodamine, lanthanide phosphors), enzymatic labels (for example, horseradish peroxidase, luciferase, alkaline phosphatase); chemiluminescent markers; biotinyl groups; predetermined polypeptide epitopes recognized by a secondary reporter (for example, leucine zipper pair sequences, binding sites for secondary antibodies, metal binding domains, epitope tags); and magnetic agents, such as gadolinium chelates. Representative examples of labels commonly employed for immunoassays include moieties that produce light, for example, acridinium compounds, and moieties that produce fluorescence, for example, fluorescein. In some embodiments, the moiety itself may not be detectably labeled but may become detectable upon reaction with yet another moiety.

The term “conjugate” refers to an antibody that is chemically linked to a second chemical moiety, such as a therapeutic or cytotoxic agent. The term “agent” includes a chemical compound, a mixture of chemical compounds, a biological macromolecule, or an extract made from biological materials. In some embodiments, the therapeutic or cytotoxic agents include, but are not limited to, pertussis toxin, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. When employed in the context of an immunoassay, the conjugate antibody may be a detectably labeled antibody used as the detection antibody.

The term “amplification” refers to the process of producing one or more copies of a nucleic acid sequence or its complement. Amplification may be linear or exponential (e.g., PCR).

The technique of “polymerase chain reaction” or “PCR” as used herein generally refers to a procedure wherein a specific region of nucleic acid, such as RNA and/or DNA, is amplified as described, for example, in U.S. Pat. No. 4,683,195. Generally, oligonucleotide primers are designed the hybridize to opposite strands of the template to be amplified, a desired distance apart. PCR can be used to amplify specific RNA sequences, specific DNA sequences from total genomic DNA, and cDNA transcribed from total cellular RNA, bacteriophage or plasmid sequences, etc.

“Quantitative real time PCR” or “qRT-PCR” refers to a form of PCR wherein the PCR is performed such that the amounts, or relative amounts of the amplified product can be quantified. This technique has been described in various publications including Cronin et al., Am. J. Pathol. 164(1):35-42 (2004); and Ma et al., Cancer Cell 5:607-616 (2004).

The term “target sequence,” “target nucleic acid,” or “target nucleic acid sequence” refers generally to a polynucleotide sequence of interest, e.g., a polynucleotide sequence that is targeted for amplification using, for example, qRT-PCR.

The term “detection” includes any means of detecting, including direct and indirect detection.

The term “prediction” is used herein to refer to the likelihood that a subject will respond either favorably or unfavorably to a therapeutic agent or combination of therapeutic agents. In some embodiments, the prediction relates to the extent of those responses. In some embodiments, the methods of prediction described herein can be used to make treatment decisions by choosing the most appropriate treatment modalities for a particular subject.

### II. Anti-ICOS Antibodies

Antibodies directed against ICOS are provided. Anti-ICOS antibodies include, but are not limited to, humanized antibodies, chimeric antibodies, mouse antibodies, human antibodies, and antibodies comprising the heavy chain and/or light chain CDRs discussed herein. In some embodiments, an isolated antibody that binds to ICOS is provided. In some embodiments, a monoclonal antibody that binds to ICOS is provided. In some embodiments, an anti-ICOS antibody is an agonist anti-ICOS antibody. In some embodiments, administration of the anti-ICOS antibodies described herein increases the number of Teff cells; activates Teff cells; depletes Treg cells in a subject; and/or increases the ratio of Teff cells to Treg cells. In some embodiments, the Treg cells are CD4+ FoxP3+ T cells. In some embodiments, the Teff cells are CD8+ T cells. In some embodiments, the Teff cells are CD4+ FoxP3− T cells and CD8+ T cells.

In some embodiments, the anti-ICOS antibody is an agonist antibody. In certain preferred embodiments, the agonist anti-ICOS antibody is the 37A10S713 antibody described below (e.g., an antibody having light and heavy chain sequences corresponding to SEQ ID NOs: 189 and 188, respectively, or SEQ ID NOs: 189 and 216, respectively).

In some embodiments, the anti-ICOS antibody comprises six CDRs including (a) HCDR1 comprising the amino acid sequence of SEQ ID NO: 62; (b) HCDR2 comprising the amino acid sequence of SEQ ID NO: 63; (c) HCDR3 comprising the amino acid sequence of SEQ ID NO: 64; (d) LCDR1 comprising the amino acid sequence of SEQ ID NO: 65; (e) LCDR2 comprising the amino acid sequence of SEQ ID NO: 66; and (f) LCDR3 comprising the amino acid sequence of SEQ ID NO: 67.

In some embodiments, an anti-ICOS antibody comprises a heavy chain variable region and a light chain variable region.

In some embodiments, the anti-ICOS antibody comprises six CDRs including (a) HCDR1 comprising the amino acid sequence of SEQ ID NO: 72; (b) HCDR2 comprising the amino acid sequence of SEQ ID NO: 73; (c) HCDR3 comprising the amino acid sequence of SEQ ID NO: 74; (d) LCDR1 comprising the amino acid sequence of SEQ ID NO: 75; (e) LCDR2 comprising the amino acid sequence of SEQ ID NO: 76; and (f) LCDR3 comprising the amino acid sequence of SEQ ID NO: 77.

In some embodiments, the anti-ICOS antibody comprises six CDRs including (a) HCDR1 comprising the amino acid sequence of SEQ ID NO: 82; (b) HCDR2 comprising the amino acid sequence of SEQ ID NO: 83; (c) HCDR3 comprising the amino acid sequence of SEQ ID NO: 84; (d) LCDR1 comprising the amino acid sequence of SEQ ID NO: 85; (e) LCDR2 comprising the amino acid sequence of SEQ ID NO: 86; and (f) LCDR3 comprising the amino acid sequence of SEQ ID NO: 87.

In some embodiments, the anti-ICOS antibody comprises six CDRs including (a) HCDR1 comprising the amino acid sequence of SEQ ID NO: 92; (b) HCDR2 comprising the amino acid sequence of SEQ ID NO: 93; (c) HCDR3 comprising the amino acid sequence of SEQ ID NO: 94; (d) LCDR1 comprising the amino acid sequence of SEQ ID NO: 95; (e) LCDR2 comprising the amino acid sequence of SEQ ID NO: 96; and (f) LCDR3 comprising the amino acid sequence of SEQ ID NO: 97.

In some embodiments, the anti-ICOS antibody comprises six CDRs including (a) HCDR1 comprising the amino acid sequence of SEQ ID NO: 102; (b) HCDR2 comprising the amino acid sequence of SEQ ID NO: 103; (c) HCDR3 comprising the amino acid sequence of SEQ ID NO: 104; (d) LCDR1 comprising the amino acid sequence of SEQ ID NO: 105; (e) LCDR2 comprising the amino acid sequence of SEQ ID NO: 106; and (f) LCDR3 comprising the amino acid sequence of SEQ ID NO: 107.

In some embodiments, the anti-ICOS antibody comprises six CDRs including (a) HCDR1 comprising the amino acid sequence of SEQ ID NO: 112; (b) HCDR2 comprising the amino acid sequence of SEQ ID NO: 113; (c) HCDR3 comprising the amino acid sequence of SEQ ID NO: 114; (d) LCDR1 comprising the amino acid sequence of SEQ ID NO: 115; (e) LCDR2 comprising the amino acid sequence of SEQ ID NO: 116; and (f) LCDR3 comprising the amino acid sequence of SEQ ID NO: 117.

In some embodiments, the anti-ICOS antibody comprises at least one, at least two, or all three VH CDR sequences selected from (a) HCDR1 comprising the amino acid sequence of SEQ ID NO: 62; (b) HCDR2 comprising the amino acid sequence of SEQ ID NO: 63; and (c) HCDR3 comprising the amino acid sequence of SEQ ID NO: 64.

In some embodiments, the anti-ICOS antibody comprises at least one, at least two, or all three VH CDR sequences selected from (a) HCDR1 comprising the amino acid sequence of SEQ ID NO: 72; (b) HCDR2 comprising the amino acid sequence of SEQ ID NO: 73; and (c) HCDR3 comprising the amino acid sequence of SEQ ID NO: 74.

In some embodiments, the anti-ICOS antibody comprises at least one, at least two, or all three VH CDR sequences selected from (a) HCDR1 comprising the amino acid sequence of SEQ ID NO: 82; (b) HCDR2 comprising the amino acid sequence of SEQ ID NO: 83; and (c) HCDR3 comprising the amino acid sequence of SEQ ID NO: 84.

In some embodiments, the anti-ICOS antibody comprises at least one, at least two, or all three VH CDR sequences selected from (a) HCDR1 comprising the amino acid sequence of SEQ ID NO: 92; (b) HCDR2 comprising the amino acid sequence of SEQ ID NO: 93; and (c) HCDR3 comprising the amino acid sequence of SEQ ID NO: 94.

In some embodiments, the anti-ICOS antibody comprises at least one, at least two, or all three VH CDR sequences selected from (a) HCDR1 comprising the amino acid sequence of SEQ ID NO: 102; (b) HCDR2 comprising the amino acid sequence of SEQ ID NO: 103; and (c) HCDR3 comprising the amino acid sequence of SEQ ID NO: 104.

In some embodiments, the anti-ICOS antibody comprises at least one, at least two, or all three VH CDR sequences selected from (a) HCDR1 comprising the amino acid sequence of SEQ ID NO: 112; (b) HCDR2 comprising the amino acid sequence of SEQ ID NO: 113; and (c) HCDR3 comprising the amino acid sequence of SEQ ID NO: 114.

In some embodiments, the antibody comprises at least one, at least two, or all three VL CDR sequences selected from (a) LCDR1 comprising the amino acid sequence of SEQ ID NO: 65; (b) LCDR2 comprising the amino acid sequence of SEQ ID NO: 66; and (c) LCDR3 comprising the amino acid sequence of SEQ ID NO: 67.

In some embodiments, the antibody comprises at least one, at least two, or all three VL CDR sequences selected from (a) LCDR1 comprising the amino acid sequence of SEQ ID NO: 75; (b) LCDR2 comprising the amino acid sequence of SEQ ID NO: 76; and (c) LCDR3 comprising the amino acid sequence of SEQ ID NO: 77.

In some embodiments, the antibody comprises at least one, at least two, or all three VL CDR sequences selected from (a) LCDR1 comprising the amino acid sequence of SEQ ID NO: 85; (b) LCDR2 comprising the amino acid sequence of SEQ ID NO: 86; and (c) LCDR3 comprising the amino acid sequence of SEQ ID NO: 87.

In some embodiments, the antibody comprises at least one, at least two, or all three VL CDR sequences selected from (a) LCDR1 comprising the amino acid sequence of SEQ ID NO: 95; (b) LCDR2 comprising the amino acid sequence of SEQ ID NO: 96; and (c) LCDR3 comprising the amino acid sequence of SEQ ID NO: 97.

In some embodiments, the antibody comprises at least one, at least two, or all three VL CDR sequences selected from (a) LCDR1 comprising the amino acid sequence of SEQ ID NO: 105; (b) LCDR2 comprising the amino acid sequence of SEQ ID NO: 106; and (c) LCDR3 comprising the amino acid sequence of SEQ ID NO: 107.

In some embodiments, the antibody comprises at least one, at least two, or all three VL CDR sequences selected from (a) LCDR1 comprising the amino acid sequence of SEQ ID NO: 115; (b) LCDR2 comprising the amino acid sequence of SEQ ID NO: 116; and (c) LCDR3 comprising the amino acid sequence of SEQ ID NO: 117.

In some embodiments, the anti-ICOS antibody comprises (I) a VH domain comprising at least one, at least two, or all three VH CDR sequences selected from (a) HCDR1 comprising the amino acid sequence of SEQ ID NO: 62; (b) HCDR2 comprising the amino acid sequence of SEQ ID NO: 63; and (c) HCDR3 comprising the amino acid sequence of SEQ ID NO: 64; and (II) a VL domain comprising at least one, at least two, or all three VL CDR sequences selected from (d) LCDR1 comprising the amino acid sequence of SEQ ID NO: 65; (e) LCDR2 comprising the amino acid sequence of SEQ ID NO: 66; and (f) LCDR3 comprising the amino acid sequence of SEQ ID NO: 67.

In some embodiments, the anti-ICOS antibody comprises (I) a VH domain comprising at least one, at least two, or all three VH CDR sequences selected from (a) HCDR1 comprising the amino acid sequence of SEQ ID NO: 72; (b) HCDR2 comprising the amino acid sequence of SEQ ID NO: 73; and (c) HCDR3 comprising the amino acid sequence of SEQ ID NO: 74; and (II) a VL domain comprising at least one, at least two, or all three VL CDR sequences selected from (d) LCDR1 comprising the amino acid sequence of SEQ ID NO: 75; (e) LCDR2 comprising the amino acid sequence of SEQ ID NO: 76; and (f) LCDR3 comprising the amino acid sequence of SEQ ID NO: 77.

In some embodiments, the anti-ICOS antibody comprises (I) a VH domain comprising at least one, at least two, or all three VH CDR sequences selected from (a) HCDR1 comprising the amino acid sequence of SEQ ID NO: 82; (b) HCDR2 comprising the amino acid sequence of SEQ ID NO: 83; and (c) HCDR3 comprising the amino acid sequence of SEQ ID NO: 84; and

(II) a VL domain comprising at least one, at least two, or all three VL CDR sequences selected from (d) LCDR1 comprising the amino acid sequence of SEQ ID NO: 85; (e) LCDR2 comprising the amino acid sequence of SEQ ID NO: 86; and (f) LCDR3 comprising the amino acid sequence of SEQ ID NO: 87.

In some embodiments, the anti-ICOS antibody comprises (I) a VH domain comprising at least one, at least two, or all three VH CDR sequences selected from (a) HCDR1 comprising the amino acid sequence of SEQ ID NO: 92; (b) HCDR2 comprising the amino acid sequence of SEQ ID NO: 93; and (c) HCDR3 comprising the amino acid sequence of SEQ ID NO: 94; and (II) a VL domain comprising at least one, at least two, or all three VL CDR sequences selected from (d) LCDR1 comprising the amino acid sequence of SEQ ID NO: 95; (e) LCDR2 comprising the amino acid sequence of SEQ ID NO: 96; and (f) LCDR3 comprising the amino acid sequence of SEQ ID NO: 97.

In some embodiments, the anti-ICOS antibody comprises (I) a VH domain comprising at least one, at least two, or all three VH CDR sequences selected from (a) HCDR1 comprising the amino acid sequence of SEQ ID NO: 102; (b) HCDR2 comprising the amino acid sequence of SEQ ID NO: 103; and (c) HCDR3 comprising the amino acid sequence of SEQ ID NO: 104; and (II) a VL domain comprising at least one, at least two, or all three VL CDR sequences selected from (d) LCDR1 comprising the amino acid sequence of SEQ ID NO: 105; (e) LCDR2 comprising the amino acid sequence of SEQ ID NO: 106; and (f) LCDR3 comprising the amino acid sequence of SEQ ID NO: 107.

In some embodiments, the anti-ICOS antibody comprises (I) a VH domain comprising at least one, at least two, or all three VH CDR sequences selected from (a) HCDR1 comprising the amino acid sequence of SEQ ID NO: 112; (b) HCDR2 comprising the amino acid sequence of SEQ ID NO: 113; and (c) HCDR3 comprising the amino acid sequence of SEQ ID NO: 114; and (II) a VL domain comprising at least one, at least two, or all three VL CDR sequences selected from (d) LCDR1 comprising the amino acid sequence of SEQ ID NO: 115; (e) LCDR2 comprising the amino acid sequence of SEQ ID NO: 116; and (f) LCDR3 comprising the amino acid sequence of SEQ ID NO: 117.

In some embodiments, the anti-ICOS antibody comprises (I) a VH domain comprising at least one, at least two, or all three VH CDR sequences selected from (a) HCDR1 comprising the amino acid sequence of SEQ ID NO: 194; (b) HCDR2 comprising the amino acid sequence of SEQ ID NO: 195; and (c) HCDR3 comprising the amino acid sequence of SEQ ID NO: 196; and (II) a VL domain comprising at least one, at least two, or all three VL CDR sequences selected from (d) LCDR1 comprising the amino acid sequence of SEQ ID NO: 197; (e) LCDR2 comprising the amino acid sequence of SEQ ID NO: 198; and (f) LCDR3 comprising the amino acid sequence of SEQ ID NO: 199.

In some embodiments, the anti-ICOS antibody comprises (I) a VH domain comprising at least one, at least two, or all three VH CDR sequences selected from (a) HCDR1 comprising the amino acid sequence of SEQ ID NO: 202; (b) HCDR2 comprising the amino acid sequence of SEQ ID NO: 203; and (c) HCDR3 comprising the amino acid sequence of SEQ ID NO: 204; and (II) a VL domain comprising at least one, at least two, or all three VL CDR sequences selected from (d) LCDR1 comprising the amino acid sequence of SEQ ID NO: 205; (e) LCDR2 comprising the amino acid sequence of SEQ ID NO: 206; and (f) LCDR3 comprising the amino acid sequence of SEQ ID NO: 207.

In some embodiments, the anti-ICOS antibody comprises (I) a VH domain comprising at least one, at least two, or all three VH CDR sequences selected from (a) HCDR1 comprising the amino acid sequence of SEQ ID NO: 210; (b) HCDR2 comprising the amino acid sequence of SEQ ID NO: 211; and (c) HCDR3 comprising the amino acid sequence of SEQ ID NO: 212; and (II) a VL domain comprising at least one, at least two, or all three VL CDR sequences selected from (d) LCDR1 comprising the amino acid sequence of SEQ ID NO: 213; (e) LCDR2 comprising the amino acid sequence of SEQ ID NO: 214; and (f) LCDR3 comprising the amino acid sequence of SEQ ID NO: 215.

In some embodiments, an anti-ICOS antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 60, 70, 80, 90, 100, or 110. In some embodiments, an anti-ICOS antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 192, 200, or 208. In some embodiments, a VH sequence having at least 90%, 91%, 92%, 93%, 94%, 95%, 96%, 97%, 98%, or 99% identity contains substitutions (for example, conservative substitutions), insertions, or deletions relative to the reference sequence, but an anti-ICOS antibody comprising that sequence retains the ability to bind to ICOS. In some embodiments, a total of 1 to 10 amino acids have been substituted, inserted and/or deleted in SEQ ID NO: 60, 70, 80, 90, 100, or 110. In some embodiments, a total of 1 to 10 amino acids have been substituted, inserted and/or deleted in SEQ ID NO: 192, 200, or 208. In some embodiments, substitutions, insertions, or deletions occur in regions outside the CDRs (that is, in the FRs). Optionally, the anti-ICOS antibody comprises the VH sequence in SEQ ID NO: 60, 70, 80, 90, 100, or 110, including post-translational modifications of that sequence. Optionally, the anti-ICOS antibody comprises the VH sequence in SEQ ID NO: 192, 200, or 208, including post-translational modifications of that sequence.

In some embodiments, the VH comprises: (a) HCDR1 comprising the amino acid sequence of SEQ ID NO: 62; (b) HCDR2 comprising the amino acid sequence of SEQ ID NO: 63; and (c) HCDR3 comprising the amino acid sequence of SEQ ID NO: 64.

In some embodiments, the VH comprises: (a) HCDR1 comprising the amino acid sequence of SEQ ID NO: 72; (b) HCDR2 comprising the amino acid sequence of SEQ ID NO: 73; and (c) HCDR3 comprising the amino acid sequence of SEQ ID NO: 74.

In some embodiments, the VH comprises: (a) HCDR1 comprising the amino acid sequence of SEQ ID NO: 82; (b) HCDR2 comprising the amino acid sequence of SEQ ID NO: 83; and (c) HCDR3 comprising the amino acid sequence of SEQ ID NO: 84.

In some embodiments, the VH comprises: (a) HCDR1 comprising the amino acid sequence of SEQ ID NO: 92; (b) HCDR2 comprising the amino acid sequence of SEQ ID NO: 93; and (c) HCDR3 comprising the amino acid sequence of SEQ ID NO: 94.

In some embodiments, the VH comprises: (a) HCDR1 comprising the amino acid sequence of SEQ ID NO: 102; (b) HCDR2 comprising the amino acid sequence of SEQ ID NO: 103; and (c) HCDR3 comprising the amino acid sequence of SEQ ID NO: 104.

In some embodiments, the VH comprises: (a) HCDR1 comprising the amino acid sequence of SEQ ID NO: 112; (b) HCDR2 comprising the amino acid sequence of SEQ ID NO: 113; and (c) HCDR3 comprising the amino acid sequence of SEQ ID NO: 114.

In some embodiments, the VH comprises: (a) HCDR1 comprising the amino acid sequence of SEQ ID NO: 194; (b) HCDR2 comprising the amino acid sequence of SEQ ID NO: 195; and (c) HCDR3 comprising the amino acid sequence of SEQ ID NO: 196.

In some embodiments, the VH comprises: (a) HCDR1 comprising the amino acid sequence of SEQ ID NO: 202; (b) HCDR2 comprising the amino acid sequence of SEQ ID NO: 203; and (c) HCDR3 comprising the amino acid sequence of SEQ ID NO: 204.

In some embodiments, the VH comprises: (a) HCDR1 comprising the amino acid sequence of SEQ ID NO: 210; (b) HCDR2 comprising the amino acid sequence of SEQ ID NO: 211; and (c) HCDR3 comprising the amino acid sequence of SEQ ID NO: 212.

In some embodiments, an anti-ICOS antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 61, 71, 81, 91, 101, or 111. In some embodiments, an anti-ICOS antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 193, 201, or 209. In some embodiments, a VL sequence having at least 90%, 91%, 92%, 93%, 94%, 95%, 96%, 97%, 98%, or 99% identity contains substitutions (for example, conservative substitutions), insertions, or deletions relative to the reference sequence, but an anti-ICOS antibody comprising that sequence retains the ability to bind to ICOS. In some embodiments, a total of 1 to 10 amino acids have been substituted, inserted and/or deleted in SEQ ID NO: 61, 71, 81, 91, 101, or 111. In some embodiments, a total of 1 to 10 amino acids have been substituted, inserted and/or deleted in SEQ ID NO: 193, 201, or 209. In some embodiments, the substitutions, insertions, or deletions occur in regions outside the CDRs (that is, in the FRs). Optionally, the anti-ICOS antibody comprises the VL sequence in SEQ ID 61, 71, 81, 91, 101, or 111, including post-translational modifications of that sequence. Optionally, the anti-ICOS antibody comprises the VL sequence in SEQ ID NO: 193, 201, or 209, including post-translational modifications of that sequence.

In some embodiments, the VL comprises: (a) LCDR1 comprising the amino acid sequence of SEQ ID NO: 65; (b) LCDR2 comprising the amino acid sequence of SEQ ID NO: 66; and (c) LCDR3 comprising the amino acid sequence of SEQ ID NO: 67.

In some embodiments, the VL comprises: (a) LCDR1 comprising the amino acid sequence of SEQ ID NO: 75; (b) LCDR2 comprising the amino acid sequence of SEQ ID NO: 76; and (c) LCDR3 comprising the amino acid sequence of SEQ ID NO: 77.

In some embodiments, the VL comprises: (a) LCDR1 comprising the amino acid sequence of SEQ ID NO: 85; (b) LCDR2 comprising the amino acid sequence of SEQ ID NO: 86; and (c) LCDR3 comprising the amino acid sequence of SEQ ID NO: 87.

In some embodiments, the VL comprises: (a) LCDR1 comprising the amino acid sequence of SEQ ID NO: 95; (b) LCDR2 comprising the amino acid sequence of SEQ ID NO: 96; and (c) LCDR3 comprising the amino acid sequence of SEQ ID NO: 97.

In some embodiments, the VL comprises: (a) LCDR1 comprising the amino acid sequence of SEQ ID NO: 105; (b) LCDR2 comprising the amino acid sequence of SEQ ID NO: 106; and (c) LCDR3 comprising the amino acid sequence of SEQ ID NO: 107.

In some embodiments, the VL comprises: (a) LCDR1 comprising the amino acid sequence of SEQ ID NO: 115; (b) LCDR2 comprising the amino acid sequence of SEQ ID NO: 116; and (c) LCDR3 comprising the amino acid sequence of SEQ ID NO: 117.

In some embodiments, the VL comprises: (a) LCDR1 comprising the amino acid sequence of SEQ ID NO: 197; (b) LCDR2 comprising the amino acid sequence of SEQ ID NO: 198; and (c) LCDR3 comprising the amino acid sequence of SEQ ID NO: 199.

In some embodiments, the VL comprises: (a) LCDR1 comprising the amino acid sequence of SEQ ID NO: 205; (b) LCDR2 comprising the amino acid sequence of SEQ ID NO: 206; and (c) LCDR3 comprising the amino acid sequence of SEQ ID NO: 207.

In some embodiments, the VL comprises: (a) LCDR1 comprising the amino acid sequence of SEQ ID NO: 213; (b) LCDR2 comprising the amino acid sequence of SEQ ID NO: 214; and (c) LCDR3 comprising the amino acid sequence of SEQ ID NO: 215.

In some embodiments, an anti-ICOS antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 60, 70, 80, 90, 100, or 110, and a light chain variable domain (VL) having at least 90%, 91%, 92%, 93%, 94%, 95%, 96%, 97%, 98%, 99%, or 100% sequence identity to the amino acid sequence of SEQ ID NO: 61, 71, 81, 91, 101, or 111. In some embodiments, an anti-ICOS antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 192, 200, or 208 and a light chain variable domain (VL) having at least 90%, 91%, 92%, 93%, 94%, 95%, 96%, 97%, 98%, 99%, or 100% sequence identity to the amino acid sequence of SEQ ID NO: 193, 201, or 209. In some embodiments, an anti-ICOS antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 60, 70, 80, 90, 100, or 110, and a light chain variable domain (VL) having at least 90%, 91%, 92%, 93%, 94%, 95%, 96%, 97%, 98%, 99%, or 100% sequence identity to the amino acid sequence of SEQ ID NO: 61, 71, 81, 91, 101, or 111. In some embodiments, a VH sequence having at least 90%, 91%, 92%, 93%, 94%, 95%, 96%, 97%, 98%, or 99% identity contains substitutions (for example, conservative substitutions), insertions, or deletions relative to the reference sequence, and a VL sequence having at least 90%, 91%, 92%, 93%, 94%, 95%, 96%, 97%, 98%, or 99% identity contains substitutions (for example, conservative substitutions), insertions, or deletions relative to the reference sequence, but an anti-ICOS antibody comprising that sequence retains the ability to bind to ICOS. In some embodiments, a total of 1 to 10 amino acids have been substituted, inserted and/or deleted in SEQ ID NO: 60, 70, 80, 90, 100, or 110. In some embodiments, a total of 1 to 10 amino acids have been substituted, inserted and/or deleted in SEQ ID NO: 61, 71, 81, 91, 101, or 111 In some embodiments, a total of 1 to 10 amino acids have been substituted, inserted and/or deleted in SEQ ID NO: 192, 200, or 208. In some embodiments, a total of 1 to 10 amino acids have been substituted, inserted and/or deleted in SEQ ID NO: 193, 201, or 209. In some embodiments, substitutions, insertions, or deletions occur in regions outside the CDRs (that is, in the FRs). Optionally, the anti-ICOS antibody comprises the VH sequence in SEQ ID NO: 60, 70, 80, 90, 100, or 110, and the VL sequence of SEQ ID NO: 61, 71, 81, 91, 101, or 111, including post-translational modifications of one or both sequence. Optionally, the anti-ICOS antibody comprises the VH sequence in SEQ ID NO: 192, 200, or 208, and the VL sequence of SEQ ID NO: 193, 201, or 209, including post-translational modifications of one or both sequence.

In some embodiments, the anti-ICOS antibody comprises (I) a VH domain comprising: (a) HCDR1 comprising the amino acid sequence of SEQ ID NO: 62; (b) HCDR2 comprising the amino acid sequence of SEQ ID NO: 63; and (c) HCDR3 comprising the amino acid sequence of SEQ ID NO: 64; and (II) a VL domain comprising: (d) LCDR1 comprising the amino acid sequence of SEQ ID NO: 65; (e) LCDR2 comprising the amino acid sequence of SEQ ID NO: 66; and (f) LCDR3 comprising the amino acid sequence of SEQ ID NO: 67.

In some embodiments, the anti-ICOS antibody comprises (I) a VH domain comprising: (a) HCDR1 comprising the amino acid sequence of SEQ ID NO: 72; (b) HCDR2 comprising the amino acid sequence of SEQ ID NO: 73; and (c) HCDR3 comprising the amino acid sequence of SEQ ID NO: 74; and (II) a VL domain comprising: (d) LCDR1 comprising the amino acid sequence of SEQ ID NO: 75; (e) LCDR2 comprising the amino acid sequence of SEQ ID NO: 76; and (f) LCDR3 comprising the amino acid sequence of SEQ ID NO: 77.

In some embodiments, the anti-ICOS antibody comprises (I) a VH domain comprising: (a) HCDR1 comprising the amino acid sequence of SEQ ID NO: 82; (b) HCDR2 comprising the amino acid sequence of SEQ ID NO: 83; and (c) HCDR3 comprising the amino acid sequence of SEQ ID NO: 84; and (II) a VL domain comprising: (d) LCDR1 comprising the amino acid sequence of SEQ ID NO: 85; (e) LCDR2 comprising the amino acid sequence of SEQ ID NO: 86; and (f) LCDR3 comprising the amino acid sequence of SEQ ID NO: 87.

In some embodiments, the anti-ICOS antibody comprises (I) a VH domain comprising: (a) HCDR1 comprising the amino acid sequence of SEQ ID NO: 92; (b) HCDR2 comprising the amino acid sequence of SEQ ID NO: 93; and (c) HCDR3 comprising the amino acid sequence of SEQ ID NO: 94; and (II) a VL domain comprising: (d) LCDR1 comprising the amino acid sequence of SEQ ID NO: 95; (e) LCDR2 comprising the amino acid sequence of SEQ ID NO: 96; and (f) LCDR3 comprising the amino acid sequence of SEQ ID NO: 97.

In some embodiments, the anti-ICOS antibody comprises (I) a VH domain comprising: (a) HCDR1 comprising the amino acid sequence of SEQ ID NO: 102; (b) HCDR2 comprising the amino acid sequence of SEQ ID NO: 103; and (c) HCDR3 comprising the amino acid sequence of SEQ ID NO: 104; and (II) a VL domain comprising: (d) LCDR1 comprising the amino acid sequence of SEQ ID NO: 105; (e) LCDR2 comprising the amino acid sequence of SEQ ID NO: 106; and (f) LCDR3 comprising the amino acid sequence of SEQ ID NO: 107.

In some embodiments, the anti-ICOS antibody comprises (I) a VH domain comprising: (a) HCDR1 comprising the amino acid sequence of SEQ ID NO: 112; (b) HCDR2 comprising the amino acid sequence of SEQ ID NO: 113; and (c) HCDR3 comprising the amino acid sequence of SEQ ID NO: 114; and (II) a VL domain comprising: (d) LCDR1 comprising the amino acid sequence of SEQ ID NO: 115; (e) LCDR2 comprising the amino acid sequence of SEQ ID NO: 116; and (f) LCDR3 comprising the amino acid sequence of SEQ ID NO: 117.

In some embodiments, the anti-ICOS antibody comprises (I) a VH domain comprising: (a) HCDR1 comprising the amino acid sequence of SEQ ID NO: 194; (b) HCDR2 comprising the amino acid sequence of SEQ ID NO: 195; and (c) HCDR3 comprising the amino acid sequence of SEQ ID NO: 196; and (II) a VL domain comprising: (d) LCDR1 comprising the amino acid sequence of SEQ ID NO: 197; (e) LCDR2 comprising the amino acid sequence of SEQ ID NO: 198; and (f) LCDR3 comprising the amino acid sequence of SEQ ID NO: 199.

In some embodiments, the anti-ICOS antibody comprises (I) a VH domain comprising: (a) HCDR1 comprising the amino acid sequence of SEQ ID NO: 202; (b) HCDR2 comprising the amino acid sequence of SEQ ID NO: 203; and (c) HCDR3 comprising the amino acid sequence of SEQ ID NO: 204; and (II) a VL domain comprising: (d) LCDR1 comprising the amino acid sequence of SEQ ID NO: 205; (e) LCDR2 comprising the amino acid sequence of SEQ ID NO: 206; and (f) LCDR3 comprising the amino acid sequence of SEQ ID NO: 207.

In some embodiments, the anti-ICOS antibody comprises (I) a VH domain comprising: (a) HCDR1 comprising the amino acid sequence of SEQ ID NO: 210; (b) HCDR2 comprising the amino acid sequence of SEQ ID NO: 211; and (c) HCDR3 comprising the amino acid sequence of SEQ ID NO: 212; and (II) a VL domain comprising: (d) LCDR1 comprising the amino acid sequence of SEQ ID NO: 213; (e) LCDR2 comprising the amino acid sequence of SEQ ID NO: 214; and (f) LCDR3 comprising the amino acid sequence of SEQ ID NO: 215.

In some embodiments, the antibody comprises the VH and VL sequences in SEQ ID NO: 60 and SEQ ID NO: 61, respectively, including post-translational modifications of those sequences. In some embodiments, the antibody comprises the VH and VL sequences in SEQ ID NO: 70 and SEQ ID NO: 71, respectively, including post-translational modifications of those sequences. In some embodiments, the antibody comprises the VH and VL sequences in SEQ ID NO: 80 and SEQ ID NO: 81, respectively, including post-translational modifications of those sequences. In some embodiments, the antibody comprises the VH and VL sequences in SEQ ID NO: 90 and SEQ ID NO: 91, respectively, including post-translational modifications of those sequences. In some embodiments, the antibody comprises the VH and VL sequences in SEQ ID NO: 100 and SEQ ID NO: 101, respectively, including post-translational modifications of those sequences. In some embodiments, the antibody comprises the VH and VL sequences in SEQ ID NO: 110 and SEQ ID NO: 111, respectively, including post-translational modifications of those sequences. In some embodiments, the antibody comprises the VH and VL sequences in SEQ ID NO: 192 and SEQ ID NO: 193, respectively, including post-translational modifications of those sequences. In some embodiments, the antibody comprises the VH and VL sequences in SEQ ID NO: 200 and SEQ ID NO: 201, respectively, including post-translational modifications of those sequences. In some embodiments, the antibody comprises the VH and VL sequences in SEQ ID NO: 208 and SEQ ID NO: 209, respectively, including post-translational modifications of those sequences.

### Exemplary Chimeric Antibodies

In some embodiments, an antibody provided herein is a chimeric antibody. Certain chimeric antibodies are described, for example, in U.S. Pat. No. 4,816,567; and Morrison et al., (1984) Proc. Natl. Acad. Sci. USA, 81:6851-6855 (1984)). In one example, a chimeric antibody comprises a non-human variable region (for example, a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In some embodiments, a chimeric antibody described herein comprises one or more human constant regions. In some embodiments, the human heavy chain constant region is of an isotype selected from IgA, IgG, and IgD. In some embodiments, the human light chain constant region is of an isotype selected from κ and λ. In some embodiments, a chimeric antibody described herein comprises a human IgG constant region. In some embodiments, a chimeric antibody described herein comprises a human IgG4 heavy chain constant region. In some embodiments, a chimeric antibody described herein comprises a human IgG4 constant region and a human κ light chain.

As noted above, whether or not effector function is desirable may depend on the particular method of treatment intended for an antibody. Thus, in some embodiments, when effector function is desirable, a chimeric anti-ICOS antibody comprising a human IgG1 heavy chain constant region or a human IgG3 heavy chain constant region is selected. In some embodiments, when effector function is not desirable, a chimeric anti-ICOS antibody comprising a human IgG4 or IgG2 heavy chain constant region is selected.

### Exemplary Humanized Antibodies

In some embodiments, humanized antibodies that bind ICOS are provided. Humanized antibodies are useful as therapeutic molecules because humanized antibodies reduce or eliminate the human immune response as compared to non-human antibodies, which can result in an immune response to an antibody therapeutic (such as the human anti-mouse antibody (HAMA) response), and decreased effectiveness of the therapeutic.

In some embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (for example, the antibody from which the CDR residues are derived), for example, to restore or improve antibody specificity or affinity.

Humanized antibodies and methods of making them are reviewed, for example, in Almagro and Fransson, (2008) Front. Biosci. 13: 1619-1633, and are further described, for example, in Riechmann et al., (1988) Nature 332:323-329; Queen et al., (1989) Proc. Natl Acad. Sci. USA 86: 10029-10033; U.S. Pat. Nos. 5,821,337, 7,527,791, 6,982,321, and 7,087,409; Kashmiri et al., (2005) Methods 36:25-34; Padlan, (1991) Mol. Immunol. 28:489-498 (describing “resurfacing”); Dall'Acqua et al., (2005) Methods 36:43-60 (describing “FR shuffling”); and Osbourn et al., (2005) Methods 36:61-68 and Klimka et al., (2000) Br. J. Cancer, 83:252-260 (describing the “guided selection” approach to FR shuffling).

Human framework regions that can be used for humanization include but are not limited to: framework regions selected using the “best-fit” method (see, for example, Sims et al. (1993) J. Immunol. 151:2296); framework regions derived from the consensus sequence of human antibodies of a particular subgroup of light or heavy chain variable regions (see, for example, Carter et al. (1992) Proc. Natl. Acad. Sci. USA, 89:4285; and Presta et al. (1993) J. Immunol, 151:2623); human mature (somatically mutated) framework regions or human germline framework regions (see, for example, Almagro and Fransson, (2008) Front. Biosci. 13:1619-1633); and framework regions derived from screening FR libraries (see, for example, Baca et al., (1997) J. Biol. Chem. 272: 10678-10684 and Rosok et al., (1996) J. Biol. Chem. 271 :22611-22618).

Nonlimiting exemplary humanized antibodies include 37A10S713, 37A10S714, 37A10S715, 37A10S716, 37A10S717, and 37A10S718, described herein. Nonlimiting exemplary humanized antibodies also include antibodies comprising a heavy chain variable region of an antibody selected from 37A10S713, 37A10S714, 37A10S715, 37A10S716, 37A10S717, and 37A10S718 and/or a light chain variable region of an antibody selected from 37A10S713, 37A10S714, 37A10S715, 37A10S716, 37A10S717, and 37A10S718. Nonlimiting exemplary humanized antibodies include antibodies comprising a heavy chain variable region selected from SEQ ID NOs: 60, 70, 80, 90, 100, and 110, and/or a light chain variable region selected from SEQ ID NOs: 61, 71, 81, 91, 101, and 111. Exemplary humanized antibodies also include, but are not limited to, humanized antibodies comprising heavy chain CDR1, CDR2, and CDR3, and/or light chain CDR1, CDR2, and CDR3 of an antibody selected from 37A10, 37A10S713, 37A10S714, 37A10S715, 37A10S716, 37A10S717, and 37A10S718. In some embodiments, the humanized anti-ICOS antibody comprises the CDRs described above and binds to ICOS. In some embodiments, the humanized anti-ICOS antibody comprises the CDRs described above, binds to ICOS and increases the number of Teff cells and/or activates Teff cells and/or decreases the number of Treg cells and/or increases the ratio of Teff cells to Treg cells. In some embodiments, the Treg cells are CD4+ FoxP3+ T cells. In some embodiments, the Teff cells are CD8+ T cells. In some embodiments, the Teff cells are CD4+ FoxP3− T cells and CD8+ T cells.

In some embodiments, a humanized anti-ICOS antibody comprises a heavy chain CDR1, CDR2, and CDR3 and/or a light chain CDR1, CDR2, and CDR3 of an antibody selected from 37A10, 37A10S713, 37A10S714, 37A10S715, 37A10S716, 37A10S717, and 37A10S718.

In some embodiments, a humanized anti-ICOS antibody comprises a heavy chain comprising a variable region sequence that is at least 90%, at least 91%, at least 92%, at least 93%, at least 94%, at least 95%, at least 96%, at least 97%, at least 98%, or at least 99% identical to a sequence selected from SEQ ID NOs: 60, 70, 80, 90, 100, and 110, and wherein the antibody binds ICOS. In some embodiments, a humanized anti-ICOS antibody comprises a light chain comprising a variable region sequence that is at least 90%, at least 91%, at least 92%, at least 93%, at least 94%, at least 95%, at least 96%, at least 97%, at least 98%, or at least 99% identical to a sequence selected from SEQ ID NOs: 61, 71, 81, 91, 101, and 111, wherein the antibody binds ICOS. In some embodiments, a humanized anti-ICOS antibody comprises a heavy chain comprising a variable region sequence that is at least 90%, at least 91%, at least 92%, at least 93%, at least 94%, at least 95%, at least 96%, at least 97%, at least 98%, or at least 99% identical to a sequence selected from SEQ ID NOs: 60, 70, 80, 90, 100, and 110; and a light chain comprising a variable region sequence that is at least 90%, at least 91%, at least 92%, at least 93%, at least 94%, at least 95%, at least 96%, at least 97%, at least 98%, or at least 99% identical to a sequence selected from SEQ ID NOs: 61, 71, 81, 91, 101, and 111; wherein the antibody binds ICOS.

In some embodiments, any one or more of the CDR sequences provided herein are maintained, while the remaining heavy, light, or heavy and light chain region (that is, FR1, FR2, FR3, and FR4) is at least 90%, at least 91%, at least 92%, at least 93%, at least 94%, at least 95%, at least 96%, at least 97%, at least 98%, or at least 99% identical to a sequence selected from SEQ ID NOs: 60, 70, 80, 90, 100, and 110, 61, 71, 81, 91, 101, and 111. In some embodiments, any one or more of the CDR sequences provided herein are maintained, while the remaining heavy, light, or heavy and light chain region (that is, FR1, FR2, FR3, and FR4) is at least 90%, at least 91%, at least 92%, at least 93%, at least 94%, at least 95%, at least 96%, at least 97%, at least 98%, or at least 99% identical to a sequence selected from SEQ ID NOs: 60, 70, 80, 90, 100, 110, 61, 71, 81, 91, 101, and 111.

In some embodiments, a humanized anti-ICOS antibody comprises at least one of the CDRs discussed herein. That is, in some embodiments, a humanized anti-ICOS antibody comprises at least one CDR selected from a heavy chain CDR1 discussed herein, a heavy chain CDR2 discussed herein, a heavy chain CDR3 discussed herein, a light chain CDR1 discussed herein, a light chain CDR2 discussed herein, and a light chain CDR3 discussed herein. Further, in some embodiments, a humanized anti-ICOS antibody comprises at least one mutated CDR based on a CDR discussed herein, wherein the mutated CDR comprises 1, 2, 3, or 4 amino acid substitutions relative to the CDR discussed herein. In some embodiments, one or more of the amino acid substitutions are conservative amino acid substitutions. One skilled in the art can select one or more suitable conservative amino acid substitutions for a particular CDR sequence, wherein the suitable conservative amino acid substitutions are not predicted to significantly alter the binding properties of the antibody comprising the mutated CDR.

Exemplary humanized anti-ICOS antibodies also include antibodies that compete for binding to ICOS with an antibody or fragment thereof described herein. Thus, in some embodiments, a humanized anti-ICOS antibody is provided that competes for binding to ICOS with an antibody or fragment thereof selected from 37A10, 37A105713, 37A105714, 37A10S715, 37A10S716, 37A10S717, and 37A10S718. In some embodiments, a humanized anti-ICOS antibody is provided that competes for binding to ICOS with an antibody or fragment thereof selected from 37A10, 37A10S713, 37A10S714, 37A10S715, 37A10S716, 37A10S717, and 37A10S718, and increases the number of Teff cells and/or activates Teff cells and/or decreases the number of Treg cells and/or increases the ratio of Teff cells to Treg cells. In some embodiments, the Treg cells are CD4+ FoxP3+ T cells. In some embodiments, the Teff cells are CD8+ T cells. In some embodiments, the Teff cells are CD4+ FoxP3-T cells and CD8+ T cells.

In some embodiments, a humanized anti-ICOS antibody comprises a heavy chain comprising the amino acid sequence of SEQ ID NO: 188 and a light chain comprising the amino acid sequence of SEQ ID NO: 189. In some embodiments, a humanized anti-ICOS antibody comprises a heavy chain comprising the amino acid sequence of SEQ ID NO: 216 and a light chain comprising the amino acid sequence of SEQ ID NO: 189.

### Exemplary Antibody Conjugates

In some embodiments, an anti-ICOS antibody is conjugated to another molecule. In some embodiments, the additional molecule can be a detectable marker, such as a label. In some embodiments, the additional molecule can be a therapeutic molecule, such as a cytotoxic agent. In some embodiments, a label and/or a cytotoxic agent can be conjugated to the antibody. As used herein, a label is a moiety that facilitates detection of the antibody and/or facilitates detection of a molecule to which the antibody binds. Nonlimiting exemplary labels include, but are not limited to, radioisotopes, fluorescent groups, enzymatic groups, chemiluminescent groups, biotin, epitope tags, metal-binding tags, etc. One skilled in the art can select a suitable label according to the specific application.

As used herein, a cytotoxic agent is a moiety that reduces the proliferative capacity of one or more cells. A cell has reduced proliferative capacity when the cell becomes less able to proliferate, for example, because the cell undergoes apoptosis or otherwise dies, the cell fails to proceed through the cell cycle and/or fails to divide, the cell differentiates, etc. Nonlimiting exemplary cytotoxic agents include, but are not limited to, radioisotopes, toxins, and chemotherapeutic agents. One skilled in the art can select a suitable cytotoxic according to the intended application. In some embodiments, the cytotoxic agent is at least one of an anti-metabolite, an alkylating agent, an antibiotic, a growth factor, a cytokine, an anti-angiogenic agent, an anti-mitotic agent, an anthracycline, toxin, or an apoptotic agent

### Exemplary Leader Sequences

In order for some secreted proteins to express and secrete in large quantities, a leader sequence from a heterologous protein may be desirable. In some embodiments, employing heterologous leader sequences can be advantageous in that a resulting mature polypeptide can remain unaltered as the leader sequence is removed in the ER during the secretion process. The addition of a heterologous leader sequence can be useful to express and secrete some proteins.

Certain exemplary leader sequence sequences are described, for example, in the online Leader sequence Database maintained by the Department of Biochemistry, National University of Singapore. See Choo et al., BMC Bioinformatics, 6: 249 (2005); and PCT Publication No. WO 2006/081430.

### III. Antibody Expression and Production Nucleic Acid Molecules Encoding Anti-ICOS Antibodies

Nucleic acid molecules comprising polynucleotides that encode one or more chains of an anti-ICOS antibody are provided herein. In some embodiments, a nucleic acid molecule comprises a polynucleotide that encodes a heavy chain or a light chain of an anti-ICOS antibody. In some embodiments, a nucleic acid molecule comprises both a polynucleotide that encodes a heavy chain and a polynucleotide that encodes a light chain, of an anti-ICOS antibody. In some embodiments, a first nucleic acid molecule comprises a first polynucleotide that encodes a heavy chain and a second nucleic acid molecule comprises a second polynucleotide that encodes a light chain.

In some embodiments, the heavy chain and the light chain are expressed from one nucleic acid molecule, or from two separate nucleic acid molecules, as two separate polypeptides. In some embodiments, such as when an antibody is an scFv, a single polynucleotide encodes a single polypeptide comprising both a heavy chain and a light chain linked together.

In some embodiments, a polynucleotide encoding a heavy chain or light chain of an anti-ICOS antibody comprises a nucleotide sequence that encodes at least one of the CDRs provided herein. In some embodiments, a polynucleotide encoding a heavy chain or light chain of an anti-ICOS antibody comprises a nucleotide sequence that encodes at least 3 of the CDRs provided herein. In some embodiments, a polynucleotide encoding a heavy chain or light chain of an anti-ICOS antibody comprises a nucleotide sequence that encodes at least 6 of the CDRs provided herein. In some embodiments, a polynucleotide encoding a heavy chain or light chain of an anti-ICOS antibody comprises a nucleotide sequence that encodes a leader sequence, which, when translated, is located at the N terminus of the heavy chain or light chain. As discussed above, the leader sequence may be the native heavy or light chain leader sequence, or may be another heterologous leader sequence.

In some embodiments, the nucleic acid is one that encodes for any of the amino acid sequences for the antibodies in the Sequence Table herein. In some embodiments, the nucleic acid is one that is at least 80% identical to a nucleic acid encoding any of the amino acid sequences for the antibodies in the Sequence Table herein, for example, at least 80, 85, 90, 91, 92, 93, 94, 95, 96, 97, 98, or 99% identical. In some embodiments, the nucleic acid is one that hybridizes to any one or more of the nucleic acid sequences provided herein. In some of the embodiments, the hybridization is under moderate conditions. In some embodiments, the hybridization is under highly stringent conditions, such as: at least about 6×SSC and 1% SDS at 65° C., with a first wash for 10 minutes at about 42° C. with about 20% (v/v) formamide in 0.1×SSC, and with a subsequent wash with 0.2×SSC and 0.1% SDS at 65° C.

Nucleic acid molecules can be constructed using recombinant DNA techniques conventional in the art. In some embodiments, a nucleic acid molecule is an expression vector that is suitable for expression in a selected host cell. Vectors

Vectors comprising polynucleotides that encode anti-ICOS heavy chains and/or anti-ICOS light chains are provided. Vectors comprising polynucleotides that encode anti-ICOS heavy chains and/or anti-ICOS light chains are also provided. Such vectors include, but are not limited to, DNA vectors, phage vectors, viral vectors, retroviral vectors, etc. In some embodiments, a vector comprises a first polynucleotide sequence encoding a heavy chain and a second polynucleotide sequence encoding a light chain. In some embodiments, the heavy chain and light chain are expressed from the vector as two separate polypeptides. In some embodiments, the heavy chain and light chain are expressed as part of a single polypeptide, such as, for example, when the antibody is an scFv.

In some embodiments, a first vector comprises a polynucleotide that encodes a heavy chain and a second vector comprises a polynucleotide that encodes a light chain. In some embodiments, the first vector and second vector are transfected into host cells in similar amounts (such as similar molar amounts or similar mass amounts). In some embodiments, a mole- or mass-ratio of between 5:1 and 1:5 of the first vector and the second vector is transfected into host cells. In some embodiments, a mass ratio of between 1:1 and 1:5 for the vector encoding the heavy chain and the vector encoding the light chain is used. In some embodiments, a mass ratio of 1:2 for the vector encoding the heavy chain and the vector encoding the light chain is used.

In some embodiments, a vector is selected that is optimized for expression of polypeptides in CHO or CHO-derived cells, or in NSO cells. Exemplary such vectors are described, for example, in Running Deer et al., Biotechnol. Prog. 20:880-889 (2004).

### Host Cells

In some embodiments, anti-ICOS antibody heavy chains and/or anti-ICOS antibody light chains may be expressed in prokaryotic cells, such as bacterial cells; or in eukaryotic cells, such as fungal cells (such as yeast), plant cells, insect cells, and mammalian cells. Such expression may be carried out, for example, according to procedures known in the art.

Exemplary eukaryotic cells that may be used to express polypeptides include, but are not limited to, COS cells, including COS 7 cells; 293 cells, including 293-6E cells; CHO cells, including CHO-S, DG44. Lec13 CHO cells, and FUT8 CHO cells; PER.C6® cells (Crucell); and NSO cells. In some embodiments, anti-ICOS antibody heavy chains and/or anti-ICOS antibody light chains may be expressed in yeast. See, for example, U.S. Publication No. US 2006/0270045 A1. In some embodiments, a particular eukaryotic host cell is selected based on its ability to make desired post-translational modifications to the anti-ICOS antibody heavy chains and/or anti-ICOS antibody light chains. For example, in some embodiments, CHO cells produce polypeptides that have a higher level of sialylation than the same polypeptide produced in 293 cells.

Introduction of one or more nucleic acids into a desired host cell may be accomplished by any method, including but not limited to, calcium phosphate transfection, DEAE-dextran mediated transfection, cationic lipid-mediated transfection, electroporation, transduction, infection, etc. Nonlimiting exemplary methods are described, for example, in Sambrook et al., Molecular Cloning, A Laboratory Manual, 3^{rd} ed. Cold Spring Harbor Laboratory Press (2001). Nucleic acids may be transiently or stably transfected in the desired host cells, according to any suitable method.

Host cells comprising any of the polynucleotides or vectors described herein are also provided. In some embodiments, a host cell comprising an anti-ICOS antibody is provided. Any host cells capable of over-expressing heterologous DNAs can be used for the purpose of isolating the genes encoding the antibody, polypeptide or protein of interest. Non-limiting examples of mammalian host cells include but not limited to COS, HeLa, and CHO cells. See also PCT Publication No. WO 87/04462. Suitable non-mammalian host cells include prokaryotes (such as E. coli or B. subtillis) and yeast (such as S. cerevisae, S. pombe; or K lactis).

### Purification of Antibodies

Anti-ICOS antibodies can be purified by any suitable method. Such methods include, but are not limited to, the use of affinity matrices or hydrophobic interaction chromatography. Suitable affinity ligands include the ROR1 ECD and ligands that bind antibody constant regions. For example, a Protein A, Protein G, Protein A/G, or an antibody affinity column may be used to bind the constant region and to purify an anti-ICOS antibody. Hydrophobic interactive chromatography, for example, a butyl or phenyl column, may also suitable for purifying some polypeptides such as antibodies. Ion exchange chromatography (for example anion exchange chromatography and/or cation exchange chromatography) may also suitable for purifying some polypeptides such as antibodies. Mixed-mode chromatography (for example reversed phase/anion exchange, reversed phase/cation exchange, hydrophilic interaction/anion exchange, hydrophilic interaction/cation exchange, etc.) may also suitable for purifying some polypeptides such as antibodies. Many methods of purifying polypeptides are known in the art.

### Cell-Free Production of Antibodies

In some embodiments, an anti-ICOS antibody is produced in a cell-free system. Nonlimiting exemplary cell-free systems are described, for example, in Sitaraman et al., Methods Mol. Biol. 498: 229-44 (2009); Spirin, Trends Biotechnol. 22: 538-45 (2004); Endo et al., Biotechnol. Adv. 21: 695-713 (2003).

### Antibody Compositions

In some embodiments, antibodies prepared by the methods described above are provided. In some embodiments, the antibody is prepared in a host cell. In some embodiments, the antibody is prepared in a cell-free system. In some embodiments, the antibody is purified. In some embodiments, the antibody prepared in a host cell or a cell-free system is a chimeric antibody. In some embodiments, the antibody prepared in a host cell or a cell-free system is a humanized antibody. In some embodiments, the antibody prepared in a host cell or a cell-free system is a human antibody. In some embodiments, a cell culture media comprising an anti-ICOS antibody is provided. In some embodiments, a host cell culture fluid comprising an anti-ICOS antibody is provided.

In some embodiments, compositions comprising antibodies prepared by the methods described above are provided. In some embodiments, the composition comprises an antibody prepared in a host cell. In some embodiments, the composition comprises an antibody prepared in a cell-free system. In some embodiments, the composition comprises a purified antibody. In some embodiments, the composition comprises a chimeric antibody prepared in a host cell or a cell-free system. In some embodiments, the composition comprises a humanized antibody prepared in a host cell or a cell-free system. In some embodiments, the composition comprises a human antibody prepared in a host cell or a cell-free system.

In some embodiments, a composition comprising anti-ICOS antibody at a concentration of more than about any one of 10 mg/mL, 20 mg/mL, 30 mg/mL, 40 mg/mL, 50 mg/mL, 60 mg/mL, 70 mg/mL, 80 mg/mL, 90 mg/mL, 100 mg/mL, 125 mg/mL, 150 mg/mL, 175 mg/mL, 200 mg/mL, 225 mg/mL, or 250 mg/mL is provided. In some embodiments, the composition comprises a chimeric antibody prepared in a host cell or a cell-free system. In some embodiments, the composition comprises a humanized antibody prepared in a host cell or a cell-free system. In some embodiments, the composition comprises a human antibody prepared in a host cell or a cell-free system.

### IV. Compositions and Methods Methods, Polynucleotides, Compositions, and Kits Related to Gene Expression, RNA Signatures, Identifying Subjects, and/or Predicting Responsiveness

Provided herein are methods of using the anti-ICOS antibodies, polypeptides and polynucleotides for detection, diagnosis and monitoring of a disease, disorder or condition associated with the anti-ICOS antibody epitope expression (either increased or decreased relative to a normal sample, and/or inappropriate expression, such as presence of expression in tissues(s) and/or cell(s) that normally lack the epitope expression). Provided herein are methods of determining whether a patient will respond to anti-ICOS antibody therapy. In some embodiments, methods of identifying a subject who may benefit from treatment with an anti-ICOS antibody are provided. In some embodiments, methods of predicting responsiveness of a subject with cancer to an anti-ICOS antibody are provided.

In some embodiments, the method comprises detecting whether the patient has cells that express ICOS using an anti-ICOS antibody. In some embodiments, the method of detection comprises contacting the sample with an antibody, polypeptide, or polynucleotide and determining whether the level of binding differs from that of a reference or comparison sample (such as a control). In some embodiments, the method may be useful to determine whether the antibodies or polypeptides described herein are an appropriate treatment for the subject.

In some embodiments, the cells or cell/tissue lysate are contacted with an anti-ICOS antibody and the binding between the antibody and the cell is determined. When the test cells are shown binding activity as compared to a reference cell of the same tissue type, it may indicate that the subject would benefit from treatment with an anti-ICOS antibody. In some embodiments, the test cells are from human tissues.

Various methods known in the art for detecting specific antibody-antigen binding can be used. Exemplary immunoassays which can be conducted include fluorescence polarization immunoassay (FPIA), fluorescence immunoassay (FIA), enzyme immunoassay (EIA), nephelometric inhibition immunoassay (NIA), enzyme linked immunosorbent assay (ELISA), and radioimmunoassay (MA). An indicator moiety, or label group, can be attached to the subject antibodies and is selected so as to meet the needs of various uses of the method which are often dictated by the availability of assay equipment and compatible immunoassay procedures. Appropriate labels include, without limitation, radionuclides (for example ¹²⁵I, ¹³¹I, ³⁵S, ³H, or ³²P), enzymes (for example, alkaline phosphatase, horseradish peroxidase, luciferase, or β-glactosidase), fluorescent moieties or proteins (for example, fluorescein, rhodamine, phycoerythrin, GFP, or BFP), or luminescent moieties (for example, Qdot™ nanoparticles supplied by the Quantum Dot Corporation, Palo Alto, Calif.). General techniques to be used in performing the various immunoassays noted above are known to those of ordinary skill in the art.

In some embodiments, an immunohistochemistry assay, such as an automated immunohistochemistry assay, is used to detect ICOS expressing cells, e.g., in tissue samples such as human tissue samples (e.g., tonsil tissue), which can be human normal tissue samples or human tumor tissue samples. In some embodiments, the tissue sample is an FFPE sample, such as a clinical FFPE sample.

For purposes of diagnosis, the polypeptide including antibodies can be labeled with a detectable moiety including but not limited to radioisotopes, fluorescent labels, and various enzyme-substrate labels know in the art. Methods of conjugating labels to an antibody are known in the art.

In some embodiments, the anti-ICOS antibodies need not be labeled, and the presence thereof can be detected using a second labeled antibody which binds to the first anti-ICOS antibody.

In some embodiments, the anti-ICOS antibody can be employed in any known assay method, such as competitive binding assays, direct and indirect sandwich assays, and immunoprecipitation assays. Zola, Monoclonal Antibodies: A Manual of Techniques, pp. 147-158 (CRC Press, Inc. 1987).

The anti-ICOS antibodies and polypeptides can also be used for in vivo diagnostic assays, such as in vivo imaging. Generally, the antibody or the polypeptide is labeled with a radionuclide (such as ¹¹¹In, ⁹⁹Tc, ¹⁴C, ¹³¹I, ¹²⁵I, ³H, or any other radionuclide label, including those outlined herein) so that the cells or tissue of interest can be localized using immunoscintiography.

The antibody may also be used as staining reagent in pathology using techniques well known in the art.

In some embodiments, a first antibody is used for a diagnostic and a second antibody is used as a therapeutic. In some embodiments, the first and second antibodies are different. In some embodiments, the first antibody is from a non-human, while the therapeutic is from a human. In some embodiments, the first and second antibodies can both bind to the antigen at the same time, by binding to separate epitopes.

In some embodiments, the methods provided herein comprise determining microsatellite instability (MSI) by PCR. In some embodiments, determining microsatellite instability comprises detecting genetic instability at one or more markers (loci): BAT25, BAT26, D5S346, D2S123, and D17S250. See, e.g., Boland et al., 1998, Cancer Res. 58: 5248-5257. In some embodiments, if genetic instability is detected at 2 or more of the 5 loci, the cancer is MSI-high. In some embodiments, if genetic instability is detected at 1 of the 5 loci, the cancer is MSI-low. In some embodiments, if genetic instability is detected at none of the 5 loci, the cancer is MSS.

In some embodiments, the methods provided herein comprise determining microsatellite instability (MSI) by IHC. See, e.g., AMA and NCHPEG Colorectal Cancer Fact Sheets: 11-0456:2/12:jt:Updated February 2012. In some embodiments, determining microsatellite instability comprises detecting one or more mismatch repair proteins selected from MLH1, MSH2, PMS2, and MSH6 by IHC. In some embodiments, if one or more of the mismatch repair proteins is not detected by IHC, the cancer is MSI-positive.

In some embodiments, the methods provided herein comprise measuring an mRNA level. In some embodiments, the methods provided herein comprise measuring an ICOS RNA signature, e.g., a plurality of mRNA levels that are predictive of or correlated to ICOS expression. In some embodiments, the ICOS RNA signature comprises at least two, at least three, at least four, at least five, at least six, at least seven, at least eight, at least nine, or at least ten mRNA levels, the mRNA levels being levels of mRNAs selected from Table 7.

Any suitable method of determining mRNA levels may be used. Methods for the evaluation of mRNAs include, for example, hybridization assays using complementary DNA probes (such as in situ hybridization using labeled riboprobes specific for target sequences, Northern blot and related techniques) and various nucleic acid amplification assays (such as RT-PCR using complementary primers specific for target sequences and other amplification type detection methods, such as, for example, branched DNA, SISBA, TMA and the like).

In some embodiments, the mRNA level is determined by quantitative RT-PCR. In some embodiments, the mRNA level is determined by digital PCR. In some embodiments, the mRNA level is determined by RNA-Seq. In some embodiments, the mRNA level is determined by RNase Protection Assay (RPA). In some embodiments, the mRNA level is determined by Northern blot. In some embodiments, the mRNA level is determined by in situ hybridization (ISH). In some embodiments, the mRNA level is determined by a method selected from quantitative RT-PCR, microarray, digital PCR, RNA-Seq, RNase Protection Assay (RPA), Northern blot, and in situ hybridization (ISH).

RNA-seq is a technique based on enumeration of RNA transcripts using next-generation sequencing methodologies. The level of an mRNA is determined using the frequency of observation of fragments of its sequence. For a review of RNA-Seq, see, e.g., Wang et al., Nat. Rev. Genet. (2009) 10:57-63.

A Northern blot involves the use of electrophoresis to separate RNA samples by size, and detection with a hybridization probe complementary to part of or the entire target sequence. For a discussion of Northern blotting see, e.g., Trayhurn, P. (1996) Northern Blotting. Pro. Nutrition Soc. 55:583-589.

Quantitative RT-PCR involves reverse-transcribing mRNA and then amplifying the cDNA with a polymerase chain reaction (PCR) which is monitored in real time, e.g., by measuring fluorescence, wherein dye signal is a readout of the amount of product. The dye can be, e.g., an intercalating dye, or a dye attached to a probe also comprising a quencher, wherein degradation of the probe releases the dye and results in fluorescence, the degradation being catalyzed by an exonuclease activity driven by product formation, as in the TaqMan® assay. In some embodiments of the invention, a method for detecting a target mRNA in a biological sample comprises producing cDNA from the sample by reverse transcription using at least one primer; amplifying the cDNA so produced using a target polynucleotide as sense and antisense primers to amplify target cDNAs therein; and detecting the presence of the amplified target cDNA. In addition, such methods can include one or more steps that allow one to determine the levels of target mRNA in a biological sample (e.g., by simultaneously examining the levels a reference mRNA sequence, e.g., a “housekeeping” gene such as an actin family member or the reference RNAs discussed below). Optionally, the sequence of the amplified target cDNA can be determined.

In Digital PCR, a sample is partitioned into a plurality of reaction areas and PCR is conducted in the areas. The number of areas that are positive, i.e., in which detectable product formation occurs, can be used to determine the level of the target sequence in the original sample.

In an RPA, a sample is contacted with a probe under hybridization conditions and then with a single-stranded RNA nuclease. Formation of double-stranded complexes of probe with target protect the probe from degradation, such that the amount of probe remaining can be used to determine the level of the target.

In ISH, a cell or tissue sample is contacted with a probe that hybridizes to a target RNA and hybridization is detected to determine the level of the target.

In some embodiments, methods include protocols which examine or detect mRNAs, such as target mRNAs, in a tissue or cell sample by microarray technologies. Using nucleic acid microarrays, test and control mRNA samples from test and control tissue samples are reverse transcribed and labeled to generate cDNA probes. The probes are then hybridized to an array of nucleic acids immobilized on a solid support. The array is configured such that the sequence and position of each member of the array is known. Hybridization of a labeled probe with a particular array member indicates that the sample from which the probe was derived expresses that gene. Differential gene expression analysis of disease tissue can provide valuable information. Microarray technology utilizes nucleic acid hybridization techniques and computing technology to evaluate the mRNA expression profile of thousands of genes within a single experiment (see, e.g., WO 01/75166 published Oct. 11, 2001; U.S. Pat. Nos. 5,700,637; 5,445,934; and 5,807,522; Lockart, Nature Biotechnology, 14:1675-1680 (1996); Cheung, V. G. et al., Nature Genetics 21(Suppl):15-19 (1999) for a discussion of array fabrication). DNA microarrays are miniature arrays containing gene fragments that are either synthesized directly onto or spotted onto glass or other substrates. Thousands of genes are usually represented in a single array. A typical microarray experiment involves the following steps: 1) preparation of fluorescently labeled target from RNA isolated from the sample, 2) hybridization of the labeled target to the microarray, 3) washing, staining, and scanning of the array, 4) analysis of the scanned image and 5) generation of gene expression profiles. Two types of DNA microarrays are oligonucleotide (usually 25 to 70 mers) arrays and gene expression arrays containing PCR products prepared from cDNAs. In forming an array, oligonucleotides can be either prefabricated and spotted to the surface or directly synthesized on to the surface (in situ). In some embodiments, a DNA microarray is a single-nucleotide polymorphism (SNP) microarrays, e.g., Affymetrix® SNP Array 6.0.

The Affymetrix GeneChip® system is a commercially available microarray system which comprises arrays fabricated by direct synthesis of oligonucleotides on a glass surface. Probe/Gene Arrays: Oligonucleotides, usually 25 mers, are directly synthesized onto a glass wafer by a combination of semiconductor-based photolithography and solid phase chemical synthesis technologies. Each array contains up to 400,000 different oligos and each oligo is present in millions of copies. Since oligonucleotide probes are synthesized in known locations on the array, the hybridization patterns and signal intensities can be interpreted in terms of gene identity and relative levels by the Affymetrix Microarray Suite software. Each gene is represented on the array by a series of different oligonucleotide probes. Each probe pair consists of a perfect match oligonucleotide and a mismatch oligonucleotide. The perfect match probe has a sequence exactly complimentary to the particular gene and thus measures the expression of the gene. The mismatch probe differs from the perfect match probe by a single base substitution at the center base position, disturbing the binding of the target gene transcript. This helps to determine the background and nonspecific hybridization that contributes to the signal measured for the perfect match oligo. The Microarray Suite software subtracts the hybridization intensities of the mismatch probes from those of the perfect match probes to determine the absolute or specific intensity value for each probe set. Probes are chosen based on current information from Genbank and other nucleotide repositories. The sequences are believed to recognize unique regions of the 3′ end of the gene. A GeneChip Hybridization Oven (“rotisserie” oven) is used to carry out the hybridization of up to 64 arrays at one time. The fluidics station performs washing and staining of the probe arrays. It is completely automated and contains four modules, with each module holding one probe array. Each module is controlled independently through Microarray Suite software using preprogrammed fluidics protocols. The scanner is a confocal laser fluorescence scanner which measures fluorescence intensity emitted by the labeled cRNA bound to the probe arrays. The computer workstation with Microarray Suite software controls the fluidics station and the scanner. Microarray Suite software can control up to eight fluidics stations using preprogrammed hybridization, wash, and stain protocols for the probe array. The software also acquires and converts hybridization intensity data into a presence/absence call for each gene using appropriate algorithms. Finally, the software detects changes in gene expression between experiments by comparison analysis and formats the output into .txt files, which can be used with other software programs for further data analysis.

In some embodiments, for example when quantitative RT-PCR is used, the threshold cycle number is compared between two mRNAs, and the lower threshold indicates a higher level of the respective mRNA. As a nonlimiting example, in some embodiments, if levels of ICOS mRNA and at least one reference mRNA are analyzed and the threshold cycle number (Ct) for ICOS is 28 and the Ct for the reference mRNA is 30, then ICOS is at a higher level compared to the reference. In various embodiments, similar comparisons may be carried out for any type of quantitative or semi-quantitative analytical method.

In some embodiments, the level of at least one mRNA is normalized. In some embodiments, the level of at least two mRNAs are normalized and compared to each other. In some embodiments, such normalization may allow comparison of mRNA levels when the levels are not determined simultaneously and/or in the same assay reaction. One skilled in the art can select a suitable basis for normalization, such as at least one reference mRNA or other factor, depending on the assay.

In some embodiments, the at least one reference mRNA comprises a housekeeping gene. In some embodiments, the at least one reference mRNA comprises one or more of RPLPO, PPIA, TUBB, ACTB, YMHAZ, B2M, UBC, TBP, GUSB, HPRT1, or GAPDH.

Provided herein are also polynucleotides, kits, medicines, compositions, and unit dosage forms suitable for use in any of the methods described herein.

In some embodiments, a polynucleotide provided herein is isolated. In some embodiments, a polynucleotide provided herein is detectably labeled, e.g., with a radioisotope, a fluorescent agent, or a chromogenic agent. In another embodiment, a polynucleotide is a primer. In another embodiment, a polynucleotide is an oligonucleotide, e.g., an mRNA-specific oligonucleotide. In another embodiment, an oligonucleotide may be, for example, from 7-60 nucleotides in length, 9-45 nucleotides in length, 15-30 nucleotides in length, or 18-25 nucleotides in length. In another embodiment, an oligonucleotide may be, e.g., PNA, morpholino-phosphoramidates, LNA, or 2′-alkoxyalkoxy. Polynucleotides as provided herein are useful, e.g., for the detection of target sequences, such as a sequence contained within the mRNAs in Table 7 or a reference mRNA, such as the reference mRNAs discussed above. Detection can involve hybridization, amplification, and/or sequencing, as discussed above.

In some embodiments, a composition is provided comprising a plurality of polynucleotides, the plurality comprising at least a first polynucleotide specific for a first mRNA and a second polynucleotide specific for a second mRNA, the first and second mRNAs being selected from the mRNAs in Table 7. In some embodiments, the plurality further comprises a third polynucleotide specific for a third mRNA, the third mRNA being selected from the mRNAs in Table 7. In some embodiments, the plurality further comprises a fourth polynucleotide specific for a fourth mRNA, the fourth mRNA being selected from the mRNAs in Table 7. In some embodiments, the plurality further comprises a fifth polynucleotide specific for a fifth mRNA, the fifth mRNA being selected from the mRNAs in Table 7. In some embodiments, the plurality further comprises a sixth polynucleotide specific for a sixth mRNA, the sixth mRNA being selected from the mRNAs in Table 7. In some embodiments, the plurality further comprises a seventh polynucleotide specific for a seventh mRNA, the seventh mRNA being selected from the mRNAs in Table 7. In some embodiments, the plurality further comprises an eighth polynucleotide specific for a eighth mRNA, the eighth mRNA being selected from the mRNAs in Table 7. In some embodiments, the plurality further comprises a ninth polynucleotide specific for a ninth mRNA, the ninth mRNA being selected from the mRNAs in Table 7. In some embodiments, the plurality further comprises a tenth polynucleotide specific for a tenth mRNA, the tenth mRNA being selected from the mRNAs in Table 7. It is understood that the use of ordinals (“first,” “second,” etc.) to designate polynucleotides or mRNAs indicates that the polynucleotides or mRNAs, as the case may be, are not identical to each other.

In some embodiments, a composition comprises cells or tissue obtained from a subject. In some embodiments, a composition comprises mRNA isolated from a subject. In some embodiments, a composition comprises cDNA synthesized from mRNA isolated from a subject.

In some embodiments, a composition comprises at least one polynucleotide or a plurality of polynucleotides suitable for use in detecting at least one reference mRNA. In some embodiments, a composition comprises reagents for performing hybridization and/or amplification, such as quantitative RT-PCR, microarray, digital PCR, RNA-Seq, RPA, Northern blot, or in situ hybridization ISH. Such reagents can include one or more of an enzyme with reverse transcriptase activity, a DNA polymerase (which may be thermophilic), an intercalating dye, dNTPs, buffer, a single-strand RNA nuclease, detergent, fixative (e.g., formaldehyde), cosolvent (e.g., formamide), etc.

In some embodiments, a kit is provided including one or more containers comprising at least one polynucleotide specific for an mRNA selected from the mRNAs in Table 7 or a plurality of polynucleotides, the plurality comprising at least a first polynucleotide specific for a first mRNA and a second polynucleotide specific for a second mRNA, the first and second mRNAs being selected from the mRNAs in Table 7. In some embodiments, the plurality further comprises a third polynucleotide specific for a third mRNA, the third mRNA being selected from the mRNAs in Table 7. In some embodiments, the plurality further comprises a fourth polynucleotide specific for a fourth mRNA, the fourth mRNA being selected from the mRNAs in Table 7. In some embodiments, the plurality further comprises a fifth polynucleotide specific for a fifth mRNA, the fifth mRNA being selected from the mRNAs in Table 7. In some embodiments, the plurality further comprises a sixth polynucleotide specific for a sixth mRNA, the sixth mRNA being selected from the mRNAs in Table 7. In some embodiments, the plurality further comprises a seventh polynucleotide specific for a seventh mRNA, the seventh mRNA being selected from the mRNAs in Table 7. In some embodiments, the plurality further comprises an eighth polynucleotide specific for an eighth mRNA, the eighth mRNA being selected from the mRNAs in Table 7. In some embodiments, the plurality further comprises a ninth polynucleotide specific for a ninth mRNA, the ninth mRNA being selected from the mRNAs in Table 7. In some embodiments, the plurality further comprises a tenth polynucleotide specific for a tenth mRNA, the tenth mRNA being selected from the mRNAs in Table 7. It is understood that the use of ordinals (“first,” “second,” etc.) to designate polynucleotides or mRNAs indicates that the polynucleotides or mRNAs, as the case may be, are not identical to each other. In some embodiments, the kit includes one or more containers comprising at least one polynucleotide or a plurality of polynucleotides suitable for use in detecting at least one reference mRNA. In some embodiments, the kit comprises one or more containers comprising reagents for performing hybridization and/or amplification, such as quantitative RT-PCR, microarray, digital PCR, RNA-Seq, RNase Protection Assay (RPA), Northern blot, and in situ hybridization (ISH). Such reagents can include one or more of an enzyme with reverse transcriptase activity, a DNA polymerase (which may be thermophilic), an intercalating dye, dNTPs, buffer, a single-strand RNA nuclease, detergent, fixative (e.g., formaldehyde), cosolvent (e.g., formamide), etc.

Kits can include one or more containers comprising an anti-ICOS antibody (or unit dosage forms and/or articles of manufacture). In some embodiments, a unit dosage is provided wherein the unit dosage contains a predetermined amount of a composition comprising an anti-ICOS antibody, with or without one or more additional agents. In some embodiments, such a unit dosage is supplied in single-use prefilled syringe for injection. In some embodiments, the composition contained in the unit dosage can comprise saline, sucrose, or the like; a buffer, such as phosphate, or the like; and/or be formulated within a stable and effective pH range. In some embodiments, the composition can be provided as a lyophilized powder that may be reconstituted upon addition of an appropriate liquid, for example, sterile water. In some embodiments, the composition comprises one or more substances that inhibit protein aggregation, including, but not limited to, sucrose and arginine. In some embodiments, a composition comprises heparin and/or a proteoglycan.

In some embodiments, the amount of the anti-ICOS antibody used in the unit dose can be any of the amounts provided herein for the various methods and/or compositions described.

In some embodiments, kits further comprise instructions for use in the treatment of cancer or for detection of at least one mRNA level or RNA signature in accordance with any of the methods described herein. The kit may further comprise a description of selection an individual suitable or treatment. Instructions supplied in the kits are typically written instructions on a label or package insert (for example, a paper sheet included in the kit), but machine-readable instructions (for example, instructions carried on a magnetic or optical storage disk) are also acceptable. In some embodiments, the kit further comprises two or more therapeutic agents.

The kits are in suitable packaging. Suitable packaging includes, but is not limited to, vials, bottles, jars, flexible packaging (for example, sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

### Methods of Treating Diseases Using Anti-ICOS Antibodies

Antibodies and compositions comprising antibodies are provided for use in methods of treatment for humans or animals. Methods of treating disease comprising administering anti-ICOS antibodies are also provided. Nonlimiting exemplary diseases that can be treated with anti-ICOS antibodies include, but are not limited to cancer.

In some embodiments, a method of treating a tumor is provided, wherein cells within a sample of the tumor express ICOS. In some such embodiments, the tumor may be considered to be ICOS-positive, or to express ICOS. Expression of ICOS may be determined by IHC, e.g., as discussed herein. In some embodiments, a tumor is considered to express ICOS when a sample from the tumor shows 1, 2, or 3 staining of ICOS by IHC. In some embodiments, the sample from the tumor shows 2+ or 3+ staining of ICOS by IHC. In some embodiments, a tumor sample from a subject is analyzed for ICOS expression and the subject is selected for treatment with an antibody described herein if the tumor sample shows ICOS expression. In some embodiments, the subject is selected if the tumor sample shows elevated expression of ICOS.

In some embodiments, a subject is selected for treatment with an anti-ICOS antibody provided herein if the subject's tumor is PD-L1^{LOW}. In some embodiments, a subject is selected for treatment with an anti-ICOS antibody provided herein if the subject's tumor is ICOS^{HIGH}/PD-L1^{LOW}. In some embodiments, a subject is selected for treatment with an anti-ICOS antibody provided herein if the subject's tumor is ICOS^{HIGH}/PD-L 1^{HIGH}.

The anti-ICOS antibody can be administered as needed to subjects. Determination of the frequency of administration can be made by persons skilled in the art, such as an attending physician based on considerations of the condition being treated, age of the subject being treated, severity of the condition being treated, general state of health of the subject being treated and the like. In some embodiments, an effective dose of an anti-ICOS antibody is administered to a subject one or more times. In some embodiments, the effective dose of an anti-ICOS antibody may be administered multiple times, including for periods of at least a month, at least six months, or at least a year, or at least two years.

In some embodiments, pharmaceutical compositions are administered in an amount effective for treatment of (including prophylaxis of) cancer. The therapeutically effective amount is typically dependent on the weight of the subject being treated, his or her physical or health condition, the extensiveness of the condition to be treated, or the age of the subject being treated.

Pharmaceutical compositions are administered in an amount effective for increasing the number of Teff cells; activating Teff cells; depleting Treg cells; and/or increasing the ratio of Teff cells to Treg cells. In some embodiments, the Treg cells are CD4+ FoxP3+ T cells. In some embodiments, the Teff cells are CD8+ T cells. In some embodiments, the Teff cells are CD4+ FoxP3− T cells and CD8+ T cells.

In some embodiments, treatment with anti-ICOS antibody results in a pharmacodynamics readout, such as up-regulation of ICOS ligand (ICOSL). In some embodiments, up-regulation of ICOSL is observed on the surface of B cells. In some embodiments, up-regulation of ICOSL is observed on the surface of granulocytes. In some embodiments, up-regulation of ICOSL is observed on the surface of neutrophils. Up-regulation of ICOSL may be observed on cells in the tumor; on cells in the spleen; on cells in peripheral blood. Up-regulation of ICOSL on the cell surface can be detected, for example, by flow cytometry. In some embodiments, soluble ICOSL is increased in the serum following treatment with anti-ICOS antibody. Soluble ICOSL can be detected by methods including, but not limited to, ELISA, MSD, and mass spectrometry. In some embodiments, ICOS target engagement, as measured by availability of free-receptor, by anti-ICOS antibodies may also be used as a pharmacodynamics readout. In some such embodiments, upon treatment by an anti-ICOS antibody, the number of ICOS receptors on the surface of T lymphocytes that are free to bind additional antibodies may be quantified. Decrease in observed available receptors may serve as an indication that anti-ICOS antibodies are binding their target molecule.

The therapeutically effective amount is, in some embodiments, 0.1 mg/kg or 0.3 mg/kg. In some embodiments, the therapeutically effective amount is between 0.1 mg/kg and 0.3 mg/kg.

### Pharmaceutical Compositions

In some embodiments, compositions comprising anti-ICOS antibodies are provided in formulations with a wide variety of pharmaceutically acceptable carriers (see, for example, Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7^{th} ed., Lippencott Williams and Wilkins (2004); Kibbe et al., Handbook of Pharmaceutical Excipients, 3^{rd} ed., Pharmaceutical Press (2000)). Various pharmaceutically acceptable carriers, which include vehicles, adjuvants, and diluents, are available. Moreover, various pharmaceutically acceptable auxiliary substances, such as pH adjusting and buffering agents, tonicity adjusting agents, stabilizers, wetting agents and the like, are also available. Non-limiting exemplary carriers include saline, buffered saline, dextrose, water, glycerol, ethanol, and combinations thereof.

In some embodiments, a pharmaceutical composition comprising an anti-ICOS antibody is provided. In some embodiments, the pharmaceutical composition comprises a chimeric antibody. In some embodiments, the pharmaceutical composition comprises a humanized antibody. In some embodiments, the pharmaceutical composition comprises an antibody prepared in a host cell or cell-free system as described herein. In some embodiments, the pharmaceutical composition comprises pharmaceutically acceptable carrier.

In some embodiments, pharmaceutical compositions are administered in an amount effective for treatment of (including prophylaxis of) cancer. The therapeutically effective amount is typically dependent on the weight of the subject being treated, his or her physical or health condition, the extensiveness of the condition to be treated, or the age of the subject being treated.

### Routes of Administration

In some embodiments, anti-ICOS antibodies can be administered in vivo by various routes, including, but not limited to, intravenous, intra-arterial, parenteral, intratumoral, intraperitoneal or subcutaneous. The appropriate formulation and route of administration may be selected according to the intended application.

### Combination Therapy

Anti-ICOS antibodies can be administered alone or with other modes of treatment. They can be provided before, substantially contemporaneous with, and/or after other modes of treatment, for example, surgery, chemotherapy, small-molecule targeted therapy, radiation therapy, or the administration of a biologic, such as another therapeutic antibody. In some embodiments, an anti-ICOS antibody is administered in conjunction with another anti-cancer agent.

In some embodiments, the anti-ICOS antibody is given concurrently with a second therapeutic agent. For example, the two or more therapeutic agents are administered with a time separation of no more than about 60 minutes, such as no more than about any of 30, 15, 10, 5, or 1 minutes. In some embodiments, the anti-ICOS antibody is administered sequentially with a second therapeutic agent. For example, administration of the two or more therapeutic agents are administered with a time separation of more than about 15 minutes, such as about any of 20, 30, 40, 50, or 60 minutes, 1 day, 2 days, 3 days, 1 week, 2 weeks, or 1 month, or longer.

In some embodiments, the anti-ICOS antibody is administered with a second therapeutic method for treatment. Thus, the administration of an antibody provided herein can be in combination with another system of treatment.

In some embodiments, an anti-ICOS antibody provided herein is administered with a PD-1 therapy. Exemplary PD-1 therapies include, but are not limited to, nivolumab (OPDIVO®, BMS-936558, MDX-1106, ONO-4538); pidilizumab, lambrolizumab/pembrolizumab (KEYTRUDA, MK-3475); durvalumab (anti-PD-L1 antibody, MEDI-4736; AstraZeneca/Medlmmune); RG-7446; avelumab (anti-PD-L1 antibody; MSB-0010718C; Pfizer); AMP-224; BMS-936559 (anti-PD-L1 antibody); AMP-514; MDX-1105; ANB-011; anti-LAG-3/PD-1; anti-PD-1 antibody (CoStim); anti-PD-1 antibody (Kadmon Pharm.); anti-PD-1 antibody (Immunovo); anti-TIM-3/PD-1 antibody (AnaptysBio); anti-PD-L1 antibody (CoStimNovartis); RG7446/MPDL3280A (anti-PD-L1 antibody, Genentech/Roche); KD-033, PD-1 antagonist (Agenus); STI-A1010; STI-A1110; TSR-042; and other antibodies that are directed against programmed death-1 (PD-1) or programmed death ligand 1 (PD-L1).

In some embodiments, a subject is selected for treatment with an anti-ICOS antibody provided herein and a PD-1 therapy if the subject's tumor expresses PD-L1. In some embodiments, a subject is selected for treatment with an anti-ICOS antibody provided herein and a PD-1 therapy if the subject's tumor is PD-L1^{HIGH}. In some embodiments, a subject is selected for treatment with an anti-ICOS antibody provided herein and a PD-1 therapy if the subject's tumor expresses ICOS and PD-L1. In some embodiments, a subject is selected for treatment with an anti-ICOS antibody provided herein and a PD-1 therapy if the subject's tumor is ICOS^{HIGH}/PD-L1^{HIGH}. Determining the level of PD-L1 and/or ICOS may be determined, for example, using IHC. A patient's tumor is considered to express PD-L1, in some embodiments, when 1% or more, or 5% or more, of the tumor cells in a sample show PD-L1 membrane staining by IHC. In some embodiments, more than 50% of the tumor cells in a sample show PD-L1 membrane staining by IHC. In some such embodiments, the subject's tumor is considered to be PD-L1^{HIGH}. A patient's tumor is considered to express ICOS, in some embodiments, when 1% or more of the cells in a tumor sample show ICOS staining by IHC. In some embodiments, a subject is first treated with a PD-1 therapy, and is later treated with an anti-ICOS antibody provided herein, with or without continuing the PD-1 therapy. Thus, methods provided herein include treatment of a subject with an anti-ICOS antibody, wherein the subject has previously been treated with a PD-1 therapy.

In some embodiments, the anti-ICOS antibody provided herein is administered with an agonist anti-OX40 antibody (such as Medi6469, Medlmmune; MOXR0916/RG7888, Roche). In some embodiments, the anti-ICOS antibody provided herein is administered with an anti-CTLA4 antibody (such as ipilimumab, YERVOY®, BMS; and tremelimumab, MedImmune).

In some embodiments, an anti-ICOS antibody provided herein is administered with a therapeutic antibody selected from cetuximab (such as ERBITUX®), elotuzumab (such as EMPLICITI®), rituximab (such as RITUXIN®), trastuzumab (such as HERCEPTIN®), and atezolizumab (such as TECENTRIQ®).

In some embodiments, an additional therapeutic agent is a chemotherapeutic agent. Exemplary chemotherapeutic agents that may be combined with the anti-ICOS antibodies provided herein include, but are not limited to, capecitabine, cyclophosphamide, dacarbazine, temozolomide, cyclophosphamide, docetaxel, doxorubicin, daunorubicin, cisplatin, carboplatin, epirubicin, eribulin, 5-FU, gemcitabine, irinotecan, ixabepilone, methotrexate, mitoxantrone, oxaliplatin, paclitaxel, nab-paclitaxel, ABRAXANE® (protein-bound paclitaxel), pemetrexed, vinorelbine, and vincristine. In some embodiments, an anti-ICOS antibody provided herein is administered with at least one kinase inhibitor. Nonlimiting exemplary kinase inhibitors include erlotinib, afatinib, gefitinib, crizotinib, dabrafenib, trametinib, vemurafenib, and cobimetanib.

In some embodiments, the additional therapeutic agent is an IDO inhibitor. Nonlimiting exemplary IDO inhibitors are described, e.g., in US 2016/0060237; and US 2015/0352206. Nonlimiting exemplary IDO inhibitors include Indoximod (New Link Genetics), INCB024360 (Incyte Corp), 1-methyl-D-tryptophan (New Link Genetics), and GDC-0919 (Genentech).

In some embodiments, an anti-ICOS antibody provided herein is administered in combination with an immune-modifying drug (IMiD). Nonlimiting exemplary IMiDs include thalidomide, lenalidomide, and pomalidomide.

In some embodiments, an additional therapeutic agent is a cancer vaccine. Cancer vaccines have been investigated as a potential approach for antigen transfer and activation of dendritic cells. In particular, vaccination in combination with immunologic checkpoints or agonists for co-stimulatory pathways have shown evidence of overcoming tolerance and generating increased anti-tumor response. A range of cancer vaccines have been tested that employ different approaches to promoting an immune response against the tumor (see, e.g., Emens L A, Expert Opin Emerg Drugs 13(2): 295-308 (2008)). Approaches have been designed to enhance the response of B cells, T cells, or professional antigen-presenting cells against tumors. Exemplary types of cancer vaccines include, but are not limited to, peptide-based vaccines that employ targeting distinct tumor antigens, which may be delivered as peptides/proteins or as genetically-engineered DNA vectors, viruses, bacteria, or the like; and cell biology approaches, for example, for cancer vaccine development against less well-defined targets, including, but not limited to, vaccines developed from patient-derived dendritic cells, autologous tumor cells or tumor cell lysates, allogeneic tumor cells, and the like.

Thus, in certain embodiments, the anti-ICOS antibodies provided herein may be used in combination with a cancer vaccine. Exemplary cancer vaccines include, but are not limited to, dendritic cell vaccines, oncolytic viruses, tumor cell vaccines, etc. In some embodiments, such vaccines augment the anti-tumor response. Examples of cancer vaccines that can be used in combination with anti-ICOS antibodies provided herein include, but are not limited to, MAGE3 vaccine (e.g., for melanoma and bladder cancer), MUC1 vaccine (e.g., for breast cancer), EGFRv3 (such as Rindopepimut, e.g., for brain cancer, including glioblastoma multiforme), or ALVAC-CEA (e.g., for CEA+ cancers).

Nonlimiting exemplary cancer vaccines also include Sipuleucel-T, which is derived from autologous peripheral-blood mononuclear cells (PBMCs) that include antigen-presenting cells (see, e.g., Kantoff P W et al., N Engl J Med 363:411-22 (2010)). In Sipuleucel-T generation, the patient's PBMCs are activated ex vivo with PA2024, a recombinant fusion protein of prostatic acid phosphatase (a prostate antigen) and granulocyte—macrophage colony-stimulating factor (an immune-cell activator). Another approach to a candidate cancer vaccine is to generate an immune response against specific peptides mutated in tumor tissue, such as melanoma (see, e.g., Carreno B M et al., Science 348:6236 (2015)). Such mutated peptides may, in some embodiments, be referred to as neoantigens. As a nonlimiting example of the use of neoantigens in tumor vaccines, neoantigens in the tumor predicted to bind the major histocompatibility complex protein HLA-A*02:01 are identified for individual patients with a cancer, such as melanoma. Dendritic cells from the patient are matured ex vivo, then incubated with neoantigens. The activated dendritic cells are then administered to the patient. In some embodiments, following administration of the cancer vaccine, robust T-cell immunity against the neoantigen is detectable.

In some such embodiments, the cancer vaccine is developed using a neoantigen. In some embodiments, the cancer vaccine is a DNA vaccine. In some embodiments, the cancer vaccine is an engineered virus comprising a cancer antigen, such as PROSTVAC (rilimogene galvacirepvec/rilimogene glafolivec). In some embodiments, the cancer vaccine comprises engineered tumor cells, such as GVAX, which is a granulocyte-macrophage colony-stimulating factor (GM-CSF) gene-transfected tumor cell vaccine (see, e.g., Nemunaitis, 2005, Expert Rev Vaccines, 4: 259-74).

In some embodiments, an anti-ICOS antibody described herein is administered before, concurrently, and/or after a cancer vaccine. In some embodiments, cancer vaccines developed using neoantigens are used in combination with the anti-ICOS antibodies described herein. In some such embodiments, the combination is used to treat a cancer with a high mutational burden, such as melanoma, lung, bladder, or colorectal cancer.

In some embodiments, an anti-ICOS antibody provided herein is administered in combination with a chimeric antigen receptor T cell therapy (CAR-T therapy).

In some embodiments, an anti-ICOS antibody provided herein is administered in combination with a Vascular Endothelial Growth Factor (VEGF) receptor inhibitor, such as, but not limited to, bevacizumab (Avastin®), axitinib (Inlyta®); brivanib alaninate (BMS-582664, (S)—((R)-1-(4-(4-Fluoro-2-methyl-1H-indol-5-yloxy)-5-methylpyrrolo[2,1-f][1,2,4]triazin-6-yloxy)propan-2-yl)₂-aminopropanoate); sorafenib (Nexavar®); pazopanib (Votrient®); sunitinib malate (Sutent®); cediranib (AZD2171, CAS 288383-20-1); vargatef (BIBF1120, CAS 928326-83-4); foretinib (GSK1363089); telatinib (BAY57-9352, CAS 332012-40-5); apatinib (YN968D1, CAS 811803-05-1); imatinib (Gleevec®); ponatinib (AP24534, CAS 943319-70-8); tivozanib (AV951, CAS 475108-18-0); regorafenib (BAY73-4506, CAS 755037-03-7); vatalanib dihydrochloride (PTK787, CAS 212141-51-0); brivanib (BMS-540215, CAS 649735-46-6); vandetanib (Caprelsa® or AZD6474); motesanib diphosphate (AMG706, CAS 857876-30-3, N-(2,3-dihydro-3,3-dimethyl-1H-indol-6-yl)-2-[(4-pyridinylmethyl)amino]-3-pyridinecarboxamide, described in PCT Publication No. WO 02/066470); dovitinib dilactic acid (TKI258, CAS 852433-84-2); linfanib (ABT869, CAS 796967-16-3); cabozantinib (XL184, CAS 849217-68-1); lestaurtinib (CAS 111358-88-4); N45-[[[5-(1,1-Dimethylethyl)-2-oxazolyl]methyl]thio]-2-thiazolyl]-4-piperidinecarboxamide (BMS38703, CAS 345627-80-7); (3R,4R)-4-Amino-1-((4-((3-methoxyphenyl)amino)pyrrolo[2,1-f][1,2,4]triazin-5-yl)methyl)piperidin-3-ol (BMS690514); N-(3,4-Dichloro-2-fluorophenyl)-6-methoxy-7-[[(3aα,5 β,6aα)-octahydro-2-methylcyclopenta[c]pyrrol-5-yl]methoxy]-4-quinazolinamine (XL647, CAS 781613-23-8); 4-Methyl-3-[[1-methyl-6-β-pyridinyl)-1H-pyrazolo[3,4-d]pyrimidin-4-yl]amino]-N-[3-(trifluoromethyl)phenyl]-benzamide (BHG712, CAS 940310-85-0); and aflibercept (Eylea®).

In some embodiments, an anti-ICOS antibody provided herein is administered in combination with a cytokine therapy, such as in combination with one, two, three or more cytokines. In some embodiments, the cytokine is one, two, three or more interleukins (ILs) chosen from IL-1, IL-2, IL-12, IL-15 or IL-21.

In some embodiments, an anti-ICOS antibody provided herein is administered in combination with a cytokine therapy in combination with an agent that targets PTEN. Without intending to be bound by any particular theory, it is believed that enhanced PI3K signaling reduces Treg function.

In some embodiments, an anti-ICOS antibody provided herein is administered in combination with an A2A receptor antagonist. In some embodiments, the A2aR antagonist is an A2aR pathway antagonist (e.g., a CD-73 inhibitor, such as an anti-CD73 antibody). A nonlimiting exemplary anti-CD73 antibody is MEDI9447. Without intending to be bound by any particular theory, targeting the extracellular production of adenosine by CD73 may reduce the immunosuppressive effects of adenosine MEDI9447 has been reported to have a range of activities, including, for example, inhibition of CD73 ectonucleotidase activity, relief from AMP-mediated lymphocyte suppression, and inhibition of syngeneic tumor growth. In some embodiments, an anti-ICOS antibody provided herein is administered in combination with one or more of the following: i) an agonist of Stimulator of Interferon Genes (a STING agonist), (ii) an agonist of a Toll-Like Receptor (TLR) (such as an agonist of TLR-3, -4, -5, -7, -8, or -9), (iii) a TIM-3 modulator (such as an anti-TIM-3 antibody), (iv) a VEGF receptor inhibitor, (v) a c-Met inhibitor, (vi) a TGFβ inhibitor (such as an anti-TGFβ antibody), (vii) an A2AR antagonist, and/or a (viii) BTK inhibitor.

In some embodiments, an oncolytic virus is a recombinant oncolytic virus, such as those described in US2010/0178684 A1, which is incorporated herein by reference in its entirety. In some embodiments, a recombinant oncolytic virus comprises a nucleic acid sequence (e.g., heterologous nucleic acid sequence) encoding an inhibitor of an immune or inflammatory response, e.g., as described in US2010/0178684 A1. In some embodiments, a recombinant oncolytic virus, such as oncolytic NDV, comprises a nucleic acid sequence encoding a pro-apoptotic protein (such as apoptin), a cytokine (such as GM-CSF, CSF, interferon-gamma, interleukin-2 (IL-2), or tumor necrosis factor-alpha), an immunoglobulin (such as an antibody against ED-B firbonectin), a tumor associated antigen, a bispecific adapter protein (such as a bispecific antibody or antibody fragment directed against NDV HN protein and a T cell co-stimulatory receptor, such as CD3 or CD28; or a fusion protein between human IL-2 and a single chain antibody directed against NDV HN protein). See, e.g., Zamarin et al. Future Microbiol. 7.3(2012):347-67, which is incorporated herein by reference in its entirety. In some embodiments, the oncolytic virus is a chimeric oncolytic NDV, e.g., as described in U.S. Pat. No. 8,591,881 B2, US 2012/0122185 A1, and/or US 2014/0271677 A1, each of which is incorporated herein by reference in its entirety.

In some embodiments, an oncolytic virus comprises a conditionally replicative adenovirus (CRAd), which is designed to replicate exclusively in cancer cells. See, e.g., Alemany et al. Nature Biotechnol. 18(2000):723-27, which is incorporated herein by reference in its entirety. In some embodiments, an oncolytic adenovirus comprises one described in Table 1 on page 725 of Alemany et al..

Exemplary oncolytic viruses include but are not limited to the following:
Group B Oncolytic Adenovirus (ColoAdl) (PsiOxus Therapeutics Ltd.) (see, e.g., Clinical Trial Identifier: NCT02053220);
ONCOS-102 (previously called CGTG-102), which is an adenovirus comprising granulocyte-macrophage colony stimulating factor (GM-CSF) (Oncos Therapeutics) (see, e.g., Clinical Trial Identifier: NCT01598129);
VCN-01, which is a genetically modified oncolytic human adenovirus encoding human PH20 hyaluronidase (VCN Biosciences, S.L.) (see, e.g., Clinical Trial Identifiers: NCT02045602 and NCT02045589);
Conditionally Replicative Adenovirus ICOVIR-5, which is a virus derived from wild-type human adenovirus serotype 5 (Had5) that has been modified to selectively replicate in cancer cells with a deregulated retinoblastoma/E2F pathway (Institut Catala d′Oncologia) (see, e.g., Clinical Trial Identifier: NCT01864759);
Celyvir, which comprises bone marrow-derived autologous mesenchymal stem cells (MSCs) infected with ICOVIR5, an oncolytic adenovirus (Hospital Infantil Universitario Nino Jesus, Madrid, Spain/Ramon Alemany) (see, e.g., Clinical Trial Identifier: NCT01844661); and
CG0070, which is a conditionally replicating oncolytic serotype 5 adenovirus (Ad5) in which human E2F-1 promoter drives expression of the essential Ela viral genes, thereby restricting viral replication and cytotoxicity to Rb pathway-defective tumor cells (Cold Genesys, Inc.) (see, e.g., Clinical Trial Identifier: NCT02143804); or DNX-2401 (formerly named Delta-24-RGD), which is an adenovirus that has been engineered to replicate selectively in retinoblastoma (Rb)-pathway deficient cells and to infect cells that express certain RGD-binding integrins more efficiently (Clinica Universidad de Navarra, Universidad de Navarra/DNAtrix, Inc.) (see, e.g., Clinical Trial Identifier: NCT01956734).

Exemplary BTK inhibitors include, but are not limited to, ibrutinib (PCI-32765); GDC-0834; RN-486; CGI-560; CGI-1764; HM-71224; CC-292; ONO-4059; CNX-774; or LFM-A13. In some embodiments, a BTK inhibitor does not reduce or inhibit the kinase activity of interleukin-2-inducible kinase (ITK). In some such embodiments, the BTK inhibitor is selected from GDC-0834; RN-486; CGI-560; CGI-1764; HM-71224; CC-292; ONO-4059; CNX-774; or LFM-A13. In some embodiments, a kinase inhibitor is a BTK inhibitor, such as ibrutinib (PCI-32765).

In certain embodiments, the anti-ICOS antibody is administered in combination with IL-33 and/or IL-33R inhibitors (such as, for example, an anti-IL-33 antibody or an anti-IL-33R antibody).

In certain embodiments, the anti-ICOS antibody is administered in combination with an acyl coenzyme A-cholesterol acyltransferase (ACAT) inhibitor, such as avasimibe (CI-1011).

In some embodiments, the anti-ICOS antibody is administered in combination with an inhibitor of chemokine (C-X-C motif) receptor 2 (CXCR2). In some embodiments, the CXCR2 inhibitor is danirixin (CAS Registry Number: 954126-98-8). Danirixin is also known as GSK1325756 or 1-(4-chloro-2-hydroxy-3-piperidin-3-ylsulfonylphenyl)-3-(3-fluoro-2-methylphenyl)urea, and is described, e.g., in Miller et al. Eur J Drug Metab Pharmacokinet (2014) 39:173-181; and Miller et al. BMC Pharmacology and Toxicology (2015), 16:18. In some embodiments, the CXCR2 inhibitor is reparixin (CAS Registry Number: 266359-83-5). Reparixin is also known as repertaxin or (2R)-2-[4-(2-methylpropyl)phenyl]-N-methylsulfonylpropanamide, and is a non-competitive allosteric inhibitor of CXCR1/2. Reparixin is described, e.g., in Zarbock et al. British Journal of Pharmacology (2008), 1-8. In some embodiments, the CXCR2 inhibitor is navarixin. Navarixin is also known as MK-7123, SCH 527123, PS291822, or 2-hydroxy-N,N-dimethyl-3-[[2-[[(1R)-1-(5-methylfuran-2-yl)propyl]amino]-3,4-dioxocyclobuten-1-yl]amino]benzamide, and is described, e.g., in Ning et al. Mol Cancer Ther. 2012; 11(6):1353-64.

In some embodiments, the anti-ICOS antibody is administered in combination with a CD27 agonist. In some embodiments, the CD27 agonist is varlilumab (CAS Registry Number: 1393344-72-3). Varlilumab is also known as CDX-1127 (Celldex) or 1F5, and is a fully human monoclonal antibody that targets CD27. Varlilumab activates human T cells in the context of T cell receptor stimulation and therefore mediates anti-tumor effects. Varlilumab also provides direct therapeutic effects against tumors that express CD27. Varlilumab is described, e.g., in Vitale et al., Clin Cancer Res. 2012; 18(14):3812-21, WO 2008/051424, and U.S. Pat. No. 8,481,029. In some embodiments, the CD27 agonist is BION-1402 (BioNovion), which is also known as hCD27.15. BION-1402 is an anti-human CD27 monoclonal antibody that stimulates the proliferation and/or survival of CD27+ cells. BION-1402 activates human CD27 more effectively than its ligand CD70, which results in a significantly increased effect on proliferation of CD8+ and CD4+ T-cells. BION-1402 is disclosed, e.g., as hCD27.15 in WO 2012/004367. The antibody is produced by hybridoma hCD27.15, which was deposited with the ATCC in on Jun. 2, 2010 under number PTA-11008.

In certain embodiments, the anti-ICOS antibody is administered in combination with anti-TIGIT antibodies. Examples of anti-TIGIT antibodies include OMP-313M32, BMS-986207, and the antibodies disclosed in PCT Publication Nos. WO2016028656 and WO2017053748, and U.S. Publication Nos. US20170281764 and US20160376365.

### EXAMPLES

The examples discussed below are intended to be purely exemplary of the invention and should not be considered to limit the invention in any way. The examples are not intended to represent that the experiments below are all or the only experiments performed. Efforts have been made to ensure accuracy with respect to numbers used (for example, amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

The disclosure of International Application No. PCT/US16/58032, filed Oct. 21, 2016, is incorporated by reference herein in its entirety for any purpose.

### Certain Materials and Methods

In the mouse experiments described herein, antibody 37A10S713M is an antibody that comprises the variable regions of 37A10S713 (described in the sequence table below) engineered onto a mouse IgG2a backbone.

### Sa/1N Tumor Model, Efficacy Arms

Six to eight week old female A1J mice were inoculated s.c. on the right flank with 1×10⁶ Sal/N cells in 100 ml PBS using tuberculin syringes with 27-gauge needles. Tumor growth was monitored and on day 7, animals were redistributed into new cages after normalizing the average tumor volume to 100 mm³ for each treatment group. Ten mice were included in each treatment group. Animals were administered antibody (37A10S713M or isotype control) via intraperitoneal (i.p.) injection. Dose amount (mg/kg calculated based on an average of 20 g per mouse) and number of doses are indicated in the figure legends for each experiment. Dosing was performed on days 7 and 14. Tumor growth and mouse body weights were monitored twice weekly. Mice were sacrificed when tumor volumes reached 2000 mm³ or if there were signs of clinical distress. A CrossOver group was included and assessed for both PK and efficacy.

### Pharmacokinetics (PK)/Pharmacodynamics (PD)/Receptor Availability Arms, Mice

In parallel with the efficacy arms, additional Sal/N tumor bearing mice were injected with 37A10S713M for assessment of circulating 37A10S713M antibody levels. A1J mice were inoculated subcutaneously with Sal/N cells on day −7. On day 0, mice were i.p. injected with either the isotype control mIgG2a at 2.5 mg/kg or 37A10S713M at 2.5, 0.25, or 0.05 mg/kg. A sufficient number of mice were injected to enable collection of three samples per group per time point. Blood was collected in non-treated serum separator tubes via submandibular draw for serial bleeds and cardiac puncture for terminal bleeds. The following time points were collected for serum analysis: 0, 6, 24, 48, 72, 96, 120, and 168 hrs. The first three time points were serial bleeds from the same set of mice, the next three time points were serial bleeds from a second set of mice, and the final two time points were serial bleeds from a third set of mice. Terminal time points were at 24, 96 and 168 hrs. The same bleeding scheme was repeated after a second dose of 37A10S713M was administered on day 7 post first dose. Serum was spun at 20,000×g for 10 min in serum separator tubes.

### PK Protocol

Serum levels of 37A10S713M were measured via a sandwich ELISA assay format. Plates were coated at 4° C. overnight with 0.5 μg/mL hICOS-hIgG1 diluted in coating buffer. Prior to incubation with serum, plates were washed three times and incubated for 1 hr in 1% BSA at room temperature. Serum samples were diluted 1:15 in 1% BSA with a five point 1:3 titration in duplicate. The ten point standard curve of 37A10S713M was also diluted 1:3 in duplicate. Samples were incubated on the coated and blocked plate at room temperature. Plates were washed three times. Plates were incubated for 1 hr at room temperature with 5 μg/mL of biotinylated anti-mG2a diluted in 1% BSA. Plates were washed three times. Plates were incubated for 30 min at room temperature with a 1:1000 dilution of HRP-avidin in 1% BSA. Plates were washed six times. Plates were developed with TMB Slow solution (Thermo Scientific) and the reaction was stopped with 2N H2504. Plates were read at 450 nm wavelength on a BioTek plate reader.

ADA Method

Anti-drug antibody (ADA) response to 37A10S713M was measured using a sandwich ELISA assay format. Plates were coated at 4° C. overnight with 5 μg/mL 37A10S713M diluted in coating buffer. Before incubation with serum, plates were washed three times and incubated for 1 hr in 4% non-fat skim milk at room temperature. Serum samples were diluted 1:15 in 2% non-fat skim milk in duplicate. Samples were incubated on the coated and blocked plate at room temperature for 2 hr. Plates were washed three times. Plates were incubated for 1 hr at room temperature with 2 μg/mL of anti-mIgG2a/mIgG1/mIgG2b/mIgM diluted in 2% non-fat skim milk. Plates were washed three times. Plates were incubated for 1 hr at room temperature with 2 μg/mL of secondary biotinylated anti-rat IgG. Plates were washed six times. Plates were incubated for 30 min at room temperature with a 1:1000 dilution of HRP-avidin in 2% non-fat skim milk. Plates were washed six times. Plates were developed with TMB Ultra solution (Thermo Scientific) and the reaction was stopped with 2N H2504. Plates were read at 450 nm wavelength on a BioTek plate reader. This assay provides a qualitative, but not quantitative, assessment of presence of ADA in a sample.

### Receptor Availability

In parallel with the PK assessment, blood was collected in EDTA coated microtubes via submandibular draw for serial bleeds and cardiac puncture for terminal bleeds. The following time points were collected for receptor availability analysis by flow cytometry: 0, 6, 24, 48, 72, 96, 120, and 168 hrs. The first three time points were serial bleeds from the same set of mice, the next three time points were serial bleeds from a second set of mice, and the final two time points were serial bleeds from a third set of mice. Terminal time points were at 24, 96 and 168 hrs, and tumor tissue was taken and analyzed at those time points. The same scheme was repeated after a second dose of 37A10S713M was administered on day seven post first dose.

Tumors were dissociated after a 20 min incubation with Liberase (Roche) and DNase at 37° C. Whole blood and resuspended tumor cells were blocked with 1:20 anti-mouse CD16/32 Fc block for 15 min. Cells were spun down and resuspended in extracellular antibody stain (PBS 2% FBS (Sigma)+0.05% Sodium Azide (Ricca Chemical Co.)+EDTA (Ambion)) for 30 min at 4° C. Cells were spun down and fixed and permeabilized in Foxp3 staining buffer (eBioscience) for 30 min at 4° C. Cells were spun down and resuspended in intracellular antibody stain (eBioscience) for 30 min at 4° C. Cells were spun down and resuspended in 0.1% paraformaldehyde (PFA). Cells were acquired on a BD LSRII Fortessa. Tregs were identified as live CD45+ CD3+ CD4+ Foxp3+. Teff cells were identified as live CD45+ CD3+ CD4+ Foxp3-. CD8+ cells were identified as live CD45+ CD3+ CD8+. A labeled version of the treatment antibody (37A10S713-DyLight 650) was used to detect free ICOS. Receptor availability was determined using the following formula:

$$\% Receptor Available at time t = \frac{\begin{matrix}( MFI of 37 A 10 S 713 - mG 2 aDy 650 at time t - \\ MFI of isotypeDy 650 at time t )\end{matrix}}{\begin{matrix}( MFI of 37 A 10 S 713 - mG 2 aDy 650 p r e s t u dy - \\ MFI of isotypeDy 650 prestudy )\end{matrix}} \times 100$$

### In Vitro Spike-In for Receptor Availability

Whole blood was obtained from three healthy human donors. All blood was received in Na/heparin tubes. 37A10S713 dilutions in PBS were equilibrated to 37° C., whole blood was added to the dilutions and was incubated for 4 hr at 37° C. Cells were spun and blocked with 1:20 anti-CD16/32 Fc block. Cells were spun and resuspended in antibody stain (anti-CD3, anti-CD4, 37A10S713-DyLight 650, and e780 viability dye) and incubated at 4° C. for 30 min. Cells were spun and resuspended in 0.1% PFA. Cells were acquired on a BD LSRII Fortessa. CD4+ T cells were identified as live CD3+ CD4+ cells. Receptor availability was determined using the formula:

$$\% Receptor Available at time t = \frac{\begin{matrix}( MFI of 37 A 10 S 713 - mG 2 aDy 650 at time t - \\ MFI of isotypeDy 650 at time t )\end{matrix}}{\begin{matrix}( MFI of 37 A 10 S 713 - mG 2 aDy 650 p r e s t u dy - \\ MFI of isotypeDy 650 prestudy )\end{matrix}} \times 100$$

### Toxicology Study

In a toxicology study, 37A10S713 was administered to cynomolgus monkeys by a 1 hr intravenous (i.v.) infusion at doses of 0.5, 5, and 75 mg/kg on Days 1 and 8; the animals were then terminated on Day 10. The objective of this exploratory portion of the study was:
To determine ICOS target engagement (as assessed by receptor availability) on cynomolgus monkey CD3+ CD4+ peripheral blood T cells.
To analyze changes in ICOSL surface expression on cynomolgus peripheral blood CD3-CD20+ B cells.

In a second experiment, 37A10S713 was administered via 1 hr i.v. infusion to cynolmolgus monkeys on Days 1 and 15. Blood was obtained on Day 1 (pre-study), Day 2 (24 hours post-Day 1 dose), Day 15 (before second dose), and Day 16 (24 hours post-Day 15 dose). Samples of whole blood were first Fc blocked with 5 μL Human TruStain (Biolegend) for 15 min on ice. Following Fc block, 100 μL of an antibody mix containing anti-CD3 FITC, anti-CD4 PE/Cy7, viability dye e780, and 37A10S713-DyLight 650 (or anti-RSV DyLight 650 as an isotype control). Blood/antibody mix was incubated on ice for 45 min. Following incubation, samples were centrifuged at 500×g for 5 min. Supernatant was decanted, and samples were resuspended in 200 μL of FACS staining buffer. Wash steps were repeated three times, with final resuspension in 200 μL staining buffer+0.1% paraformaldehyde.

The first two sets of samples (pre-study and 24 hours post-Day 1 dose) were shipped as one batch, and the second two sets of samples (Days 15 and 16) were shipped the following week. Samples were analyzed on a BD Fortessa flow cytometer.

For analysis of ICOS availability, staining of ICOS by DyLight 650 labeled 37A10S713 was analyzed on viable CD3+ CD4+ T cells.

### Example 1. Mouse Syngeneic Tumor Model

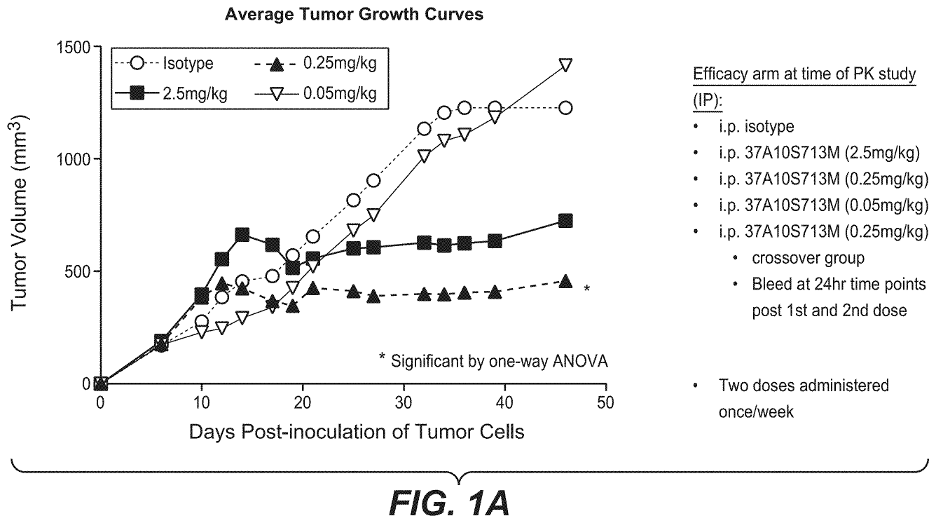

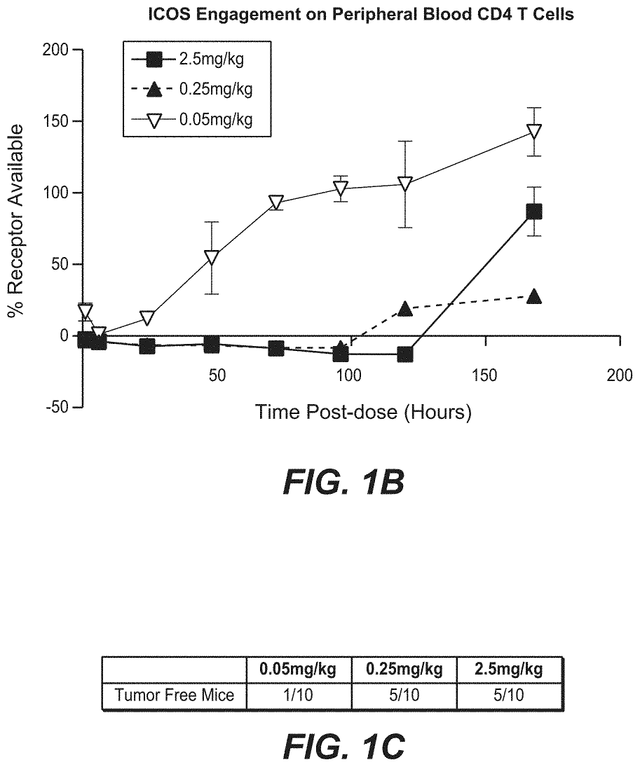

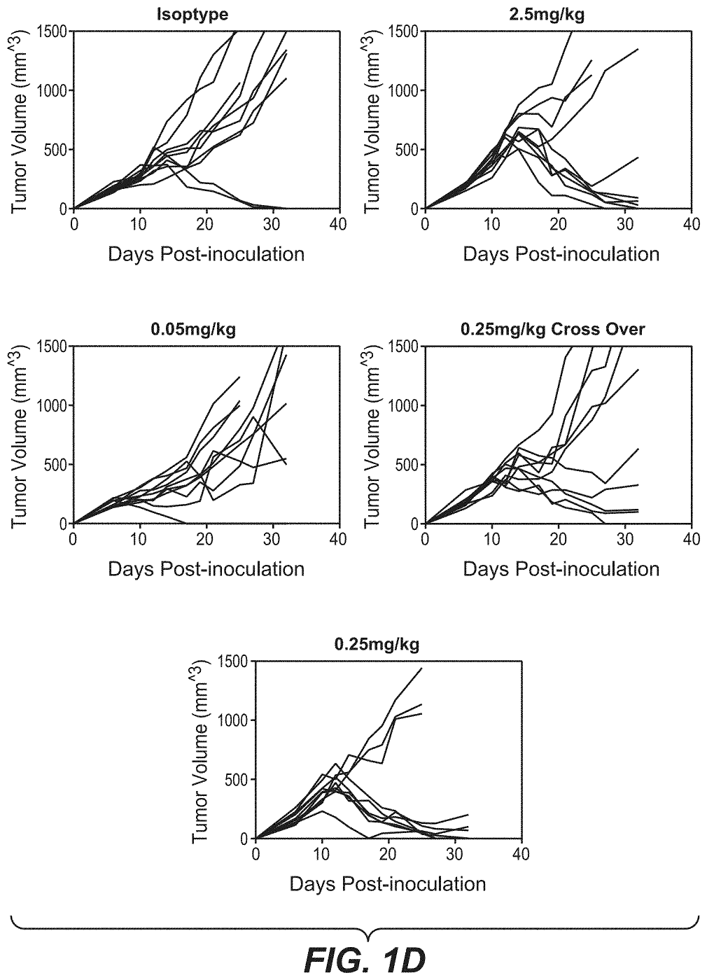

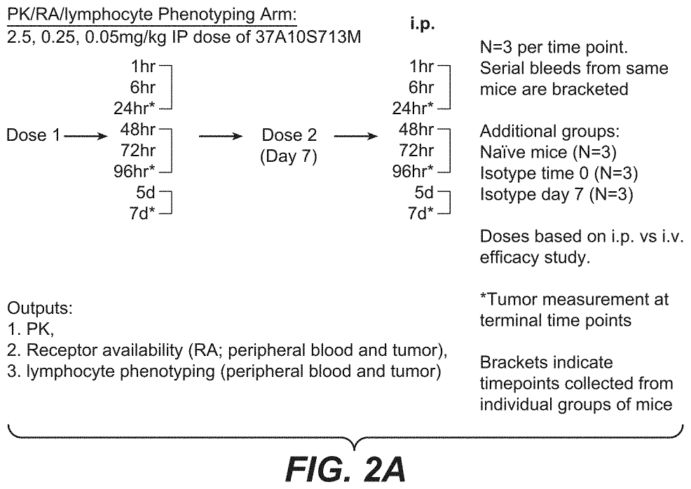

As shown in FIGS. 1A-1D, in mice treated with either 2.5 mg/kg or 0.25 mg/kg of 37A10S713M, 5 of 10 tumors showed complete regression. One additional animal in the 0.25 mg/kg had stable disease with the tumor stabilizing at about 100 mm³. In the 0.05 mg/kg 37A10S713M dose group, only 1 out of 10 tumors showed regression which was not different than the isotype control group (where 2 of 10 animals had tumor regression). Analysis of average growth curves only demonstrated a significant tumor growth inhibition in the 0.25 mg/kg dose group relative to control as determined by one-way ANOVA. The CrossOver group are mice that were assessed for both PK and efficacy.

### Example 2. Mouse PK Experiments

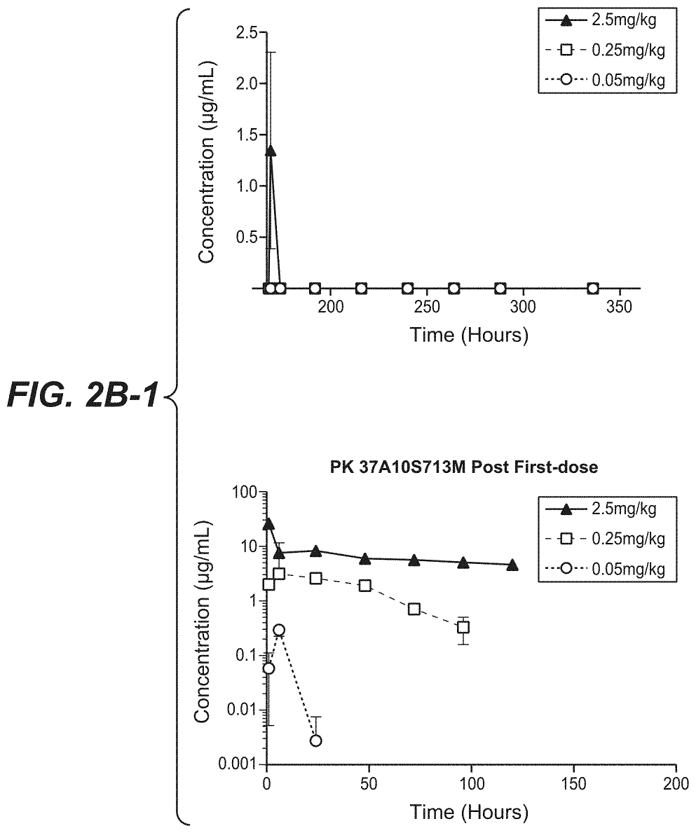

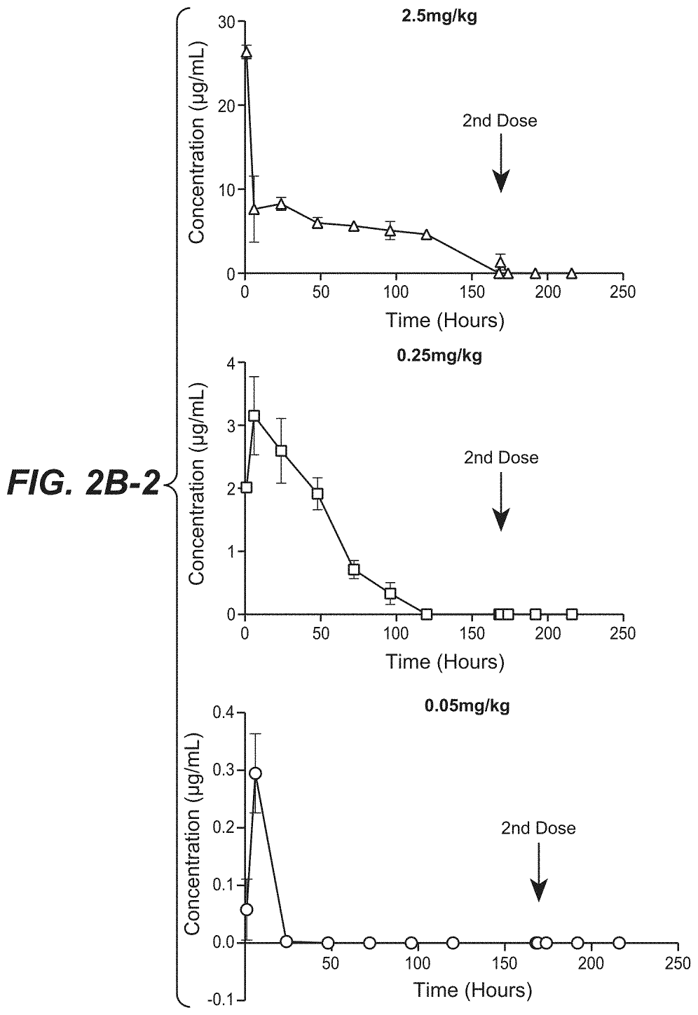

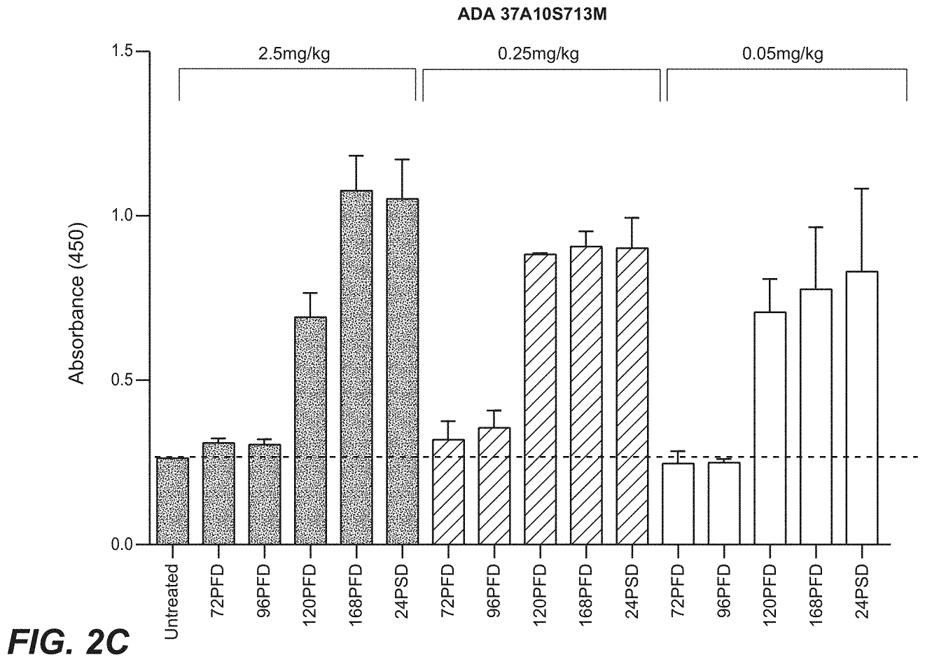

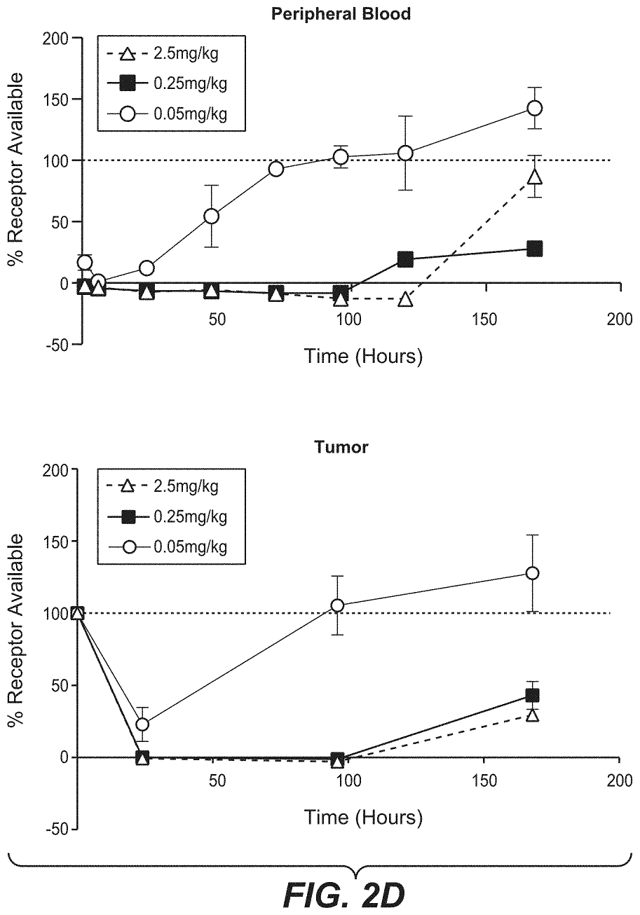

In an experiment outlined in FIG. 2A and shown in FIG. 2B, detectable levels of 37A10S713M were evident in mouse serum following i.p. administration. At the 2.5 mg/kg dose, maximum drug levels were measured in the serum at 1 hr at a concentration of approximately 26 μg/mL, with levels falling below the minimum detectable concentration of 1 ng/mL at 168 hr. At the 0.25 mg/kg dose, maximum drug levels were measured at 6 hr at a concentration of approximately 3 μg/mL and fell below detectable level at 120 hr post-first dose. At the 0.05 mg/kg dose, maximum serum drug levels were detected at 6 hr at a concentration of approximately 0.3 μg/mL and fell below the minimum level of detection at 48 hr. Isotype treated and untreated mice showed no detectable levels of 37A10S713M in the serum.

A low detectable amount of 37A10S713M was observed in the 2.5 mg/kg group at 1 hr post-second dose. At all later time points and in all the other dose groups, there was no detectable drug in the serum samples following administration of the second dose.

As shown in FIG. 2C, anti-drug antibodies (ADA) become detectable in all mice between four and five days after the initial treatment with 37A10S713M in all three dose level groups in all animals tested.

As shown in FIG. 2D, in the peripheral blood, ICOS availability decreased on total CD4 T cells following i.p. administration of 37A10S713M. After an initial dose at 2.5 mg/kg, receptor availability was found to be zero (i.e. 100% target engagement) and did not recover until after 120 hr post dosing. After an initial dose of 0.25 mg/kg, receptor availability was found to be zero and did not begin to recover until after 100 hr post dosing. After an initial dose at 0.05 mg/kg, receptor availability decreased to approximately 10% availability (90% target engagement), and began to recover by 24 hr post dosing.

In the tumor, ICOS availability was measured at the terminal time points 24, 96, and 168 hr after the initial dose. ICOS availability decreased at all dose levels on total CD4 T cells following the first dose of 37A10S713M.

### Example 3. Toxicology studies

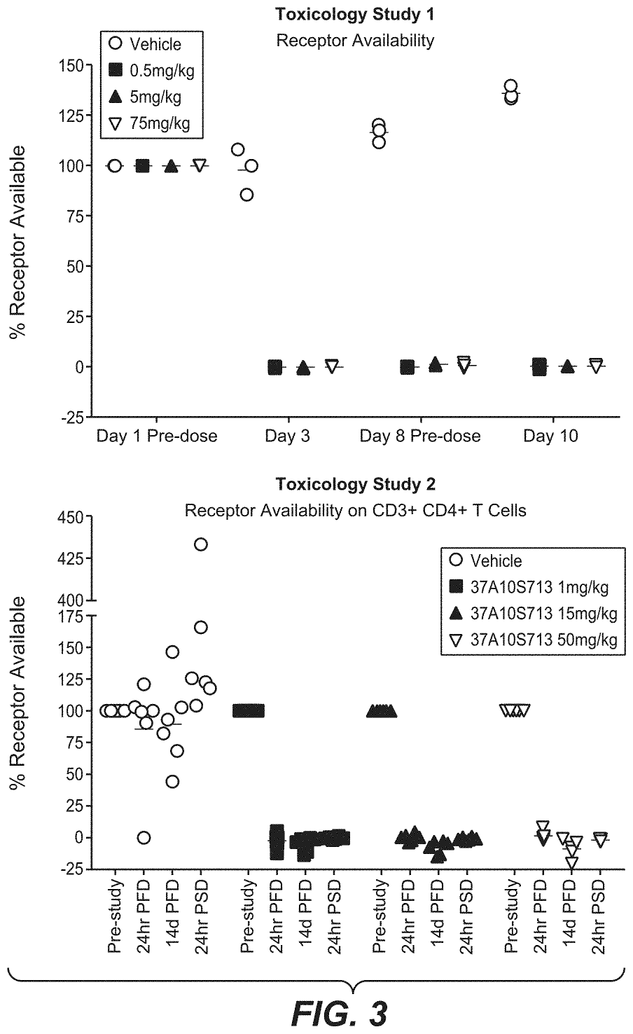

To determine ICOS target engagement, a preliminary assessment of ICOS availability on cynomolgus monkey peripheral blood T cells was performed. Following administration of 37A10S713, the ICOS was saturated, or unavailable for additional 37A10S713 binding, as revealed in the inability to detect binding of fluorescently labeled drug on viable CD3+ CD4+ T cells in all of the dose groups at all of the post-dose time points evaluated (FIG. 3, first panel).

In a second larger experiment, to determine ICOS target engagement, ICOS availability on cynomolgus monkey peripheral blood T cells was assessed. Following administration of 37A10S713, ICOS was saturated, or unavailable for additional 37A10S713 binding, as revealed by the inability to detect binding of fluorescently labeled drug on viable CD3+ CD4+ T cells in all of the dose groups at all post-dose time points (e.g., Days 2 and 16) evaluated. ICOS remained detectable at all time points in the vehicle treated group (FIG. 3, second panel).

### Example 4. QSP Model

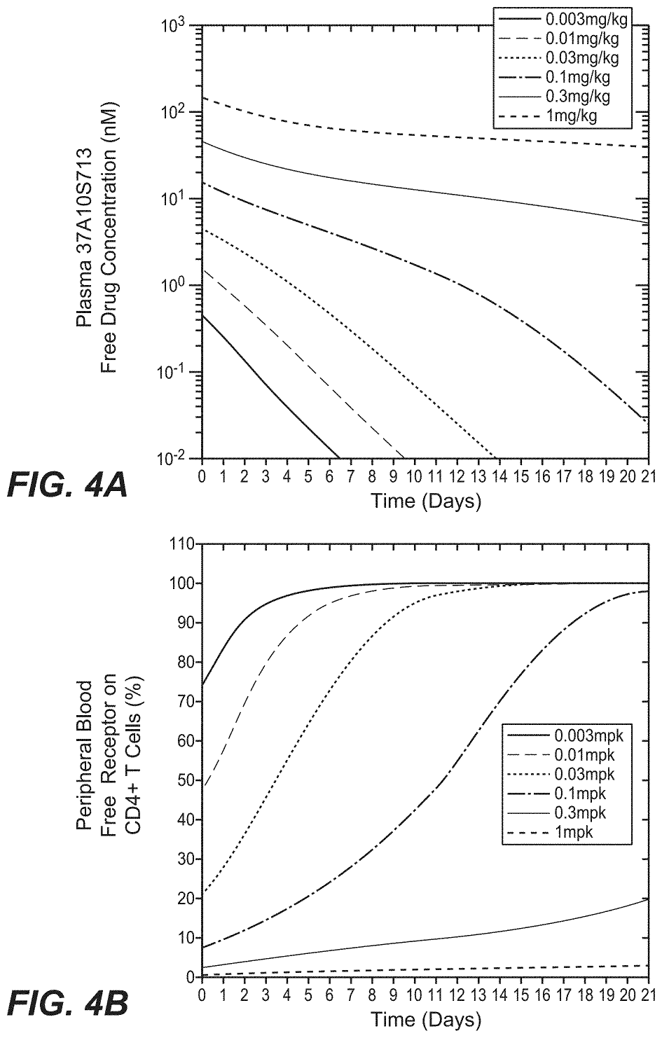

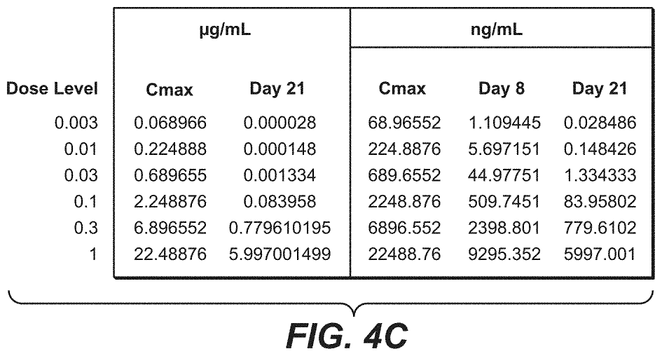

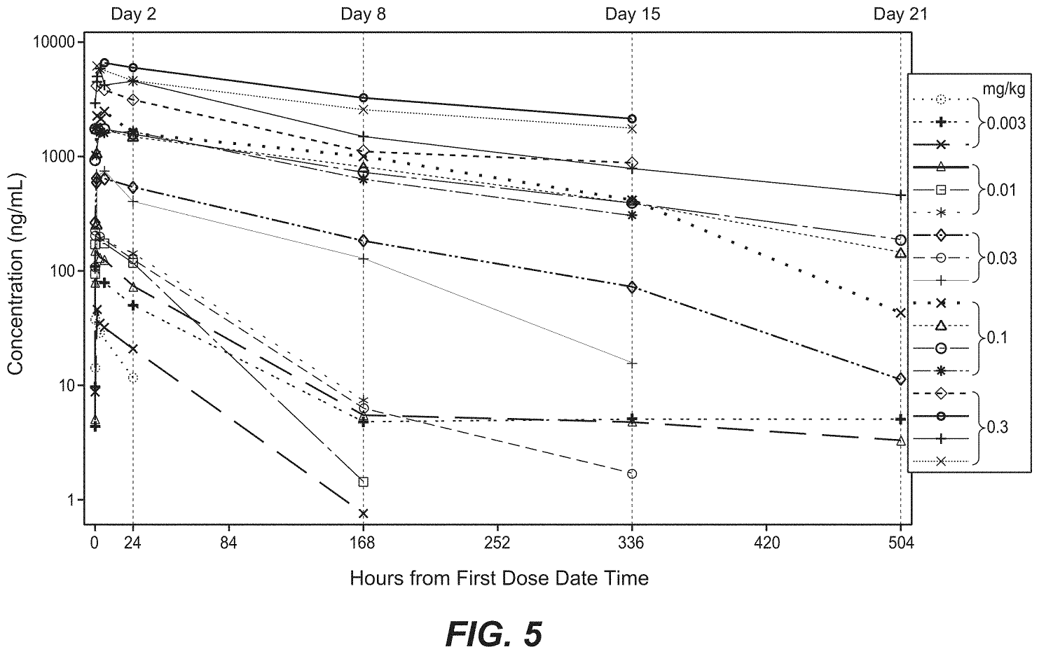

A Quantative Systems Pharmacology (QSP) model was developed for predicting the pharmacokinetics (PK) of 37A105713 and the target engagement (TE) of 37A105713 bound to ICOS in the peripheral blood and on tumor infiltrating leukocytes (TILs) in humans. This model accurately predicted the 37A105713 antibody exposures in the first cynomolgus monkey toxicology study and was in general agreement with the 37A105713 antibody exposure data from a GLP rat toxicology study (data not shown) and the second cynomolgus monkey toxicology study. PK/PD studies in mice suggest that sustained TE in peripheral blood and intra-tumor may be related to 37A10S713 antibody tumor response, and these data support selection of the maximum dose in the first in human studies based on duration (area under the effect curve, AUEC) and maximum level (Emax) of target engagement. Using this model, it was shown that doses of 37A10S713 antibody over the range of (0.003, 0.01, 0.03, 0.1, 0.3, 1 mg/kg) are predicted to cover a range of transient and low (less than 30% maximum) TE at the lowest dose and up to 21 days of complete (greater than 95%) TE at the highest dose. See FIG. 4.

### Example 5. Phase 1 Study of 37A10S713 Monotherapy and 37A10S713+ Nivolumab Combination Therapy in Solid Tumor

To be enrolled in the phase 1 study, the inclusion criteria included:
confirmed cancer that is recurrent, metastatic or persistent after at least one line of therapy and with no further standard treatment options;
at least 18 years of age;
Eastern Cooperative Oncology Group (ECOG) performance score of 0-1;
predicted life expectancy of at least 3 months;
archival tumor tissue required for all subjects;
any advanced, non-hematological, extracranial malignancy with disease progression after treatment with all available therapies known to confer clinical benefit
may have evaluable but non-measurable disease.
   The exclusion criteria included:
refused standard therapy;
history of intolerance, hypersensitivity, or treatment discontinuation due to severe immune adverse events on prior immunotherapy;
immunodeficiency;
active or prior history of autoimmune disease;
symptomatic or uncontrolled brain metastases, leptomeningeal disease, or spinal cord compression.

Study participants were administered intravenous (IV) doses of 37A10S713 of up to 1 mg/kg as a monotherapy once every 21 days, or IV doses of 37A10S713 of up to 0.3 mg/kg in combination with an IV dose of nivolumab of 240 mg once every 21 days.

Table 2 shows the demographic information and prior therapies for the subjects enrolled in the phase 1 study. Table 3 shows a summary of the disposition of the subjects enrolled in the study.

TABLE 2
Demographics and prior therapies of subjects
enrolled in phase 1 study

| | | | 37A10S713 + | | All Phase | |
|---|---|---|---|---|---|---|
| | 37A10S713 | | Nivolumab | | 1 Subjects | |
| | (N = 34) | | (N = 12) | | (N = 46) | |
| Sex, n (%) | | | | | | |
| Male | 15 | (44.1) | 2 | (16.7) | 17 | (37.0) |
| Female | 19 | (55.9) | 10 | (83.3) | 29 | (63.0) |
| Age | | | | | | |
| Mean (SD) | 60.5 | (11.34) | 59.4 | (10.90) | 60.2 | (11.12) |
| Race, n (%) | | | | | | |
| Black or African | 4 | (11.8) | 1 | (8.3) | 5 | (10.9) |
| American | | | | | | |
| White | 25 | (73.5) | 11 | (91.7) | 36 | (78.3) |
| Other | 3 | (8.8) | — | | 3 | (6.5) |
| Not Reported | 2 | (5.9) | — | | 2 | (4.3) |
| Prior Systemic Therapy | | | | | | |
| Cytotoxic | 34 | (100.0) | 11 | (91.7) | 45 | (97.8) |
| Chemotherapy | | | | | | |
| Immunotherapy | 9 | (26.5) | 7 | (58.3) | 16 | (34.8) |
| PD-1 or PD-L1 | 8 | (23.5) | 6 | (50.0) | 14 | (30.4) |
| Other therapies | 24 | (70.6) | 8 | (66.7) | 32 | (69.6) |
| Line of Prior Therapies | | | | | | |
| Median (min, max) | 4.0 | (1, 12) | 5.0 | (3, 8) | 4.0 | (1, 12) |

TABLE 3
Disposition of phase 1 study subjects
37A10S713 Monotherapy (Part A)

| mg/kg | 0.003 | 0.01 | 0.03* | 0.1 | 0.3 | 1.0 | Total |
|---|---|---|---|---|---|---|---|
| Dosed | 3 | 3 | 6 | 11 | 6 | 5 | 34 |
| Treatment | 3 (100.0) | 3 (100.0) | 4 ( 80.0) | 5 ( 45.5) | 2 (28.6) | 1 ( 20.0) | 18 ( 52.9) |
| Discontinuation | | | | | | | |
| Disease | 3 (100.0) | 3 (100.0) | 3 ( 60.0) | 3 ( 27.3) | 2 (28.6) | 1 ( 20.0) | 15 ( 44.1) |
| progression | | | | | | | |
| Adverse event | — | — | — | 1 ( 9.1) | — | — | 1 ( 2.9) |
| Subject declined | — | — | — | 1 ( 9.1) | — | — | 1 ( 2.9) |
| further participation | | | | | | | |
| Weeks on Study | 9 (0.50) | 9 (0.87) | 9(6.53) | 9(4.12) | 8 (4.08) | 4 (1.86) | 8 (4.06) |
| mean (SD) | | | | | | | |

37A10S713 + Nivolumab (Part B)

| mg/kg | 0.01 | 0.03 | 0.1 | 0.3 | Total |
|---|---|---|---|---|---|
| Dosed | 3 | 3 | 3 | 3 | 12 |
| Treatment | — | 2 ( 66.7) | — | — | 2 ( 16.7) |
| Discontinuation | | | | — | |
| Disease progression | — | 2 ( 66.7) | — | — | 2 ( 16.7) |
| [symbol] | 22 (0.93) | 10 (5.79) | 11 (0.60) | 6 (1.05) | 12 (6.58) |

*One subject was assigned to 0.03 mg/kg, but received 0.3 mg/kg for 45 minutes (equivalent to 0.225 mg/kg) on Cycle 1 day 1 and continued on 0.03 mg/kg afterwards. For safety purposes, this subject was grouped to 0.3 mg/kg. [symbol]
indicates data missing or illegible when filed

37A10S713 was dosed up to 1 mg/kg as a monotherapy and up to 0.3 mg/kg in combination with nivolumab. The maximum tolerated dose for 37A10S713 alone or in combination with nivolumab is 0.3 mg/kg. Two dose-limiting toxicities (out of 6 subjects) occurred at 1 mg/kg 37A10S713 monotherapy: one participant developed a worsening pleural effusion about 10 days after the first dose of 37A10S713; one participant developed AST/ALT 5× the upper limit of normal (ULN) about 23 days after the first dose of 37A10S713. ALT/AST returned to baseline within 72 hours after receiving prednisone 0.5 mg/kg, leading to a diagnosis of immune related hepatitis.

Treatment emergent serious adverse events were reported in eleven participants: ten participants on 37A10S713 monotherapy at 0.003 mg/kg (1); 0.1 mg/kg (4); 0.3 mg/kg (3); and 1 mg/kg (2); and one on 37A10S713 0.03 mg/kg in combination with nivolumab. Grade 3 or 4 related adverse events were reported in six participants on 37A10S713 monotherapy and no participants on 37A10S713 plus nivolumab.

Adverse events considered to be immune related but not infusion related were reported in six participants at doses of 0.03 mg/kg or above: five on 37A10S713 monotherapy and one in combination with nivolumab: alanine aminotransferase increased, blood alkaline phosphatase increased, lymphocyte count decreased, neutrophil count decreased, night sweats, pneumonitis, pruritus, rash, tumor pain, white blood cell count decreased, and diarrhea. The decreases in lymphocyte count, neutrophil count, and while blood cell count were observed in a single participant who received steroids for immune related adverse events.

Adverse events considered to be infusion related were reported in 10 participants at doses 0.003 mg/kg to 0.3 mg/kg: six on 37A10S713 monotherapy and four in combination with nivolumb: chills, pyrexia, diarrhea, hypertension, neck pain, tachycardia, nausea, vomiting, and other infusion related reactions.

One death was reported due to progressive disease after withdrawal from the study.

The adverse events are summarized in Table 4. Treatment emergent adverse events (TEAEs) include all TEAEs with a start date on or after the first dose of the study drug and on or before 28 days after the last dose of the study drug.

TABLE 4
Summary of most frequent related adverse events (>5% in any column)

| | 37A10S713 | | 37A10S713 + | | | |
|---|---|---|---|---|---|---|
| | Monotherapy (N = 34) | | Nivolumab (N = 12) | | Total (N = 46) | |
| | All AEs | Grade 3/4 | All AEs | Grade 3/4 | All AEs | Grade 3/4 |
| # TEAEs* | 153 | 17 | 40 | — | 193 | 17 |
| # Participants w. | 24 (70.6) | 11 (32.4) | 9 (75.0) | — | 33 (71.7) | 11 (23.9) |
| TEAEs, n (%) | | | | | | |
| # Participants w. | 15 (44.1) | 6 (17.6) | 6 (50.0) | — | 21 (45.7) | 6 (13.0) |
| Related TEAEs, | | | | | | |
| n (%) | | | | | | |
| Subjects w. Related | | | | | | |
| TEAEs, n (%) | | | | | | |
| Chills | 3 (8.8) | — | 2 (16.7) | — | 5 (10.9) | — |
| Nausea | 4 (11.8) | — | 1 (8.3) | — | 5 (10.9) | — |
| Decreased appetite | 3 (8.8) | — | 1 (8.3) | — | 4 (8.7) | — |
| Pyrexia | 4 (11.8) | — | — | — | 4 (8.7) | — |
| Alanine amino- | 3 (8.8) | 1 (2.9) | — | — | 3 (6.5) | 1 (2.2) |
| transferase increased | | | | | | |
| Diarrhoea | 3 (8.8) | 3 (8.8) | — | — | 3 (6.5) | 3 (6.5) |
| Fatigue | 2 (5.9) | — | 1 (8.3) | — | 3 (6.5) | — |
| Infusion related | 1 (2.9) | — | 2 (16.7) | — | 3 (6.5) | — |
| reaction | | | | | | |
| Pruritus | 3 (8.8) | — | — | — | 3 (6.5) | — |
| Aspartate amino- | 2 (5.9) | 1 (2.9) | — | — | 2 (4.3) | 1 (2.2) |
| transferase increased | | | | | | |
| Dizziness | 2 (5.9) | — | — | — | 2 (4.3) | — |
| Hypokalaemia | 1 (2.9) | 1 (2.9) | 1 (8.3) | — | 2 (4.3) | 1 (2.2) |
| Hypomagnesaemia | 2 (5.9) | — | — | — | 2 (4.3) | — |
| Vomiting | 1 (2.9) | — | 1 (8.3) | — | 2 (4.3) | — |
| Back pain | — | — | 1 (8.3) | — | 1 (2.2) | — |
| Hypothyroidism | — | — | 1 (8.3) | — | 1 (2.2) | — |

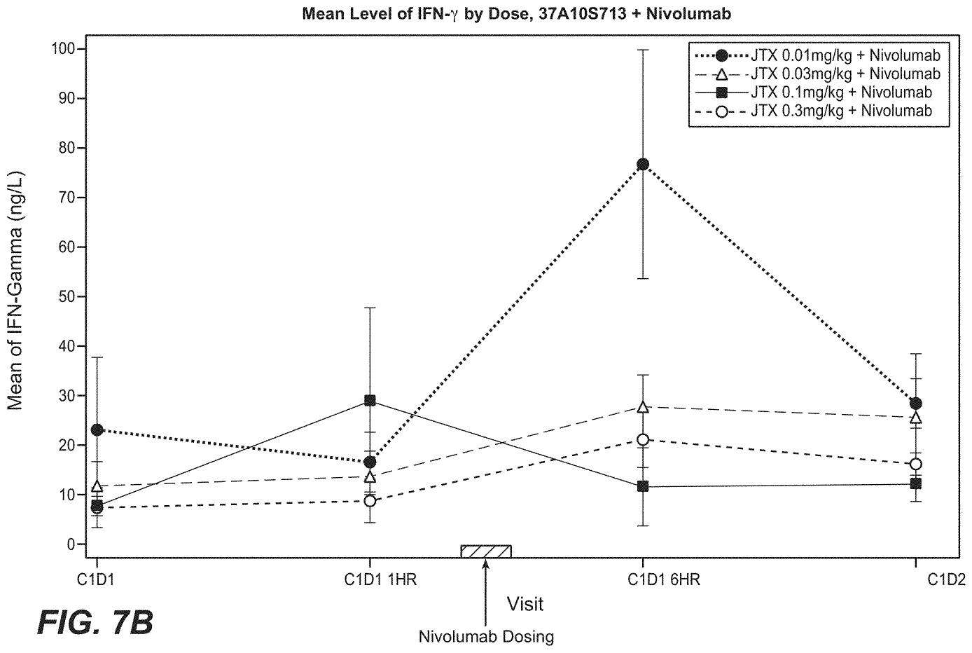

Mean levels of cytokines IFN-γ, TNFα, and IL-6 were determined in the monotherapy and combination therapy subjects. Mean increase in IFN-γ was observed at 1-6 hours at all dose levels, and may be dose related. Increases in TNF-α and IL-6 were also observed. See FIG. 7.

### Example 6. Human PK and TE Clinical Study

A summary of data regarding PK, TE, and Safety, obtained in an early human clinical study with 37A10S713 antibody, is presented in Table 5 below. Each cycle (“C”) is a single dose of 37A10S713 antibody administered at three week intervals, and the day (“D”) indicates days after administration of the dose, wherein Day 1 (D1) is the day on which the dose is administered.

TABLE 5

| | 0.1 mg/kg | 0.3 mg/kg |
|---|---|---|
| Safety | 0 out of 6 drug- | 0 out of 4 DLTs |
| | limiting toxicity | |
| | events (DLTs) | |
| Serum Conc. of | 648-978 ng/mL | 1100-3240 ng/mL |
| antibody Cycle 1 | (n = 4) | (n = 4) |
| Day 8 | | |
| Serum Conc. of | 309-412 ng/mL | 787-2120 ng/mL |
| antibody Cycle 1 | (n = 4) | (n = 4) |
| Day 15 | | |
| Serum Conc. of | 143, 185 ng/mL | >458 ng/mL |
| antibody Cycle 1 | (n = 4) | (n = 2) |
| Day 21 | | |
| TE Cycle 1 Day 8 | Not determined (ND) | >95% TE (n = 2) |
| TE Cycle 1 Day 15 | ND | >95% TE (n = 2) |
| TE Cycle 1 Day 21 | ND | >95% TE (n = 2) |
| TE data from later | Subject A | ND |
| time points from | (0.1 mg/kg) C2D1 | |
| subjects with no | some available target | |
| available baseline | Subject B | |
| data; qualitative | (0.1 mg/kg) C3D1 | |
| evaluation of | no available target | |
| presence of | Subject C | |
| available target | (0.03 mg/kg) C6D1 | |
| | substantial available | |
| | target | |
| Immunophenotyping | NA | Total CD4: no significant |
| (IP) results in serum | | change from baseline |
| (all timepoints) | | CD4 Teff: no significant |
| | | change from baseline |
| | | Tregs: No significant |
| | | change from baseline |
| | | CD8: No significant |
| | | change from baseline |

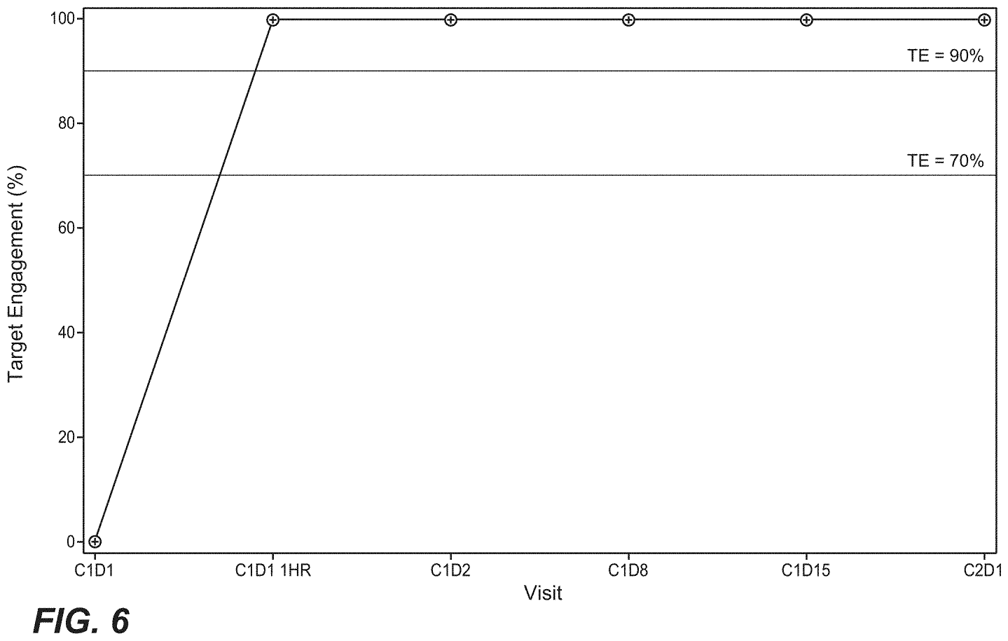

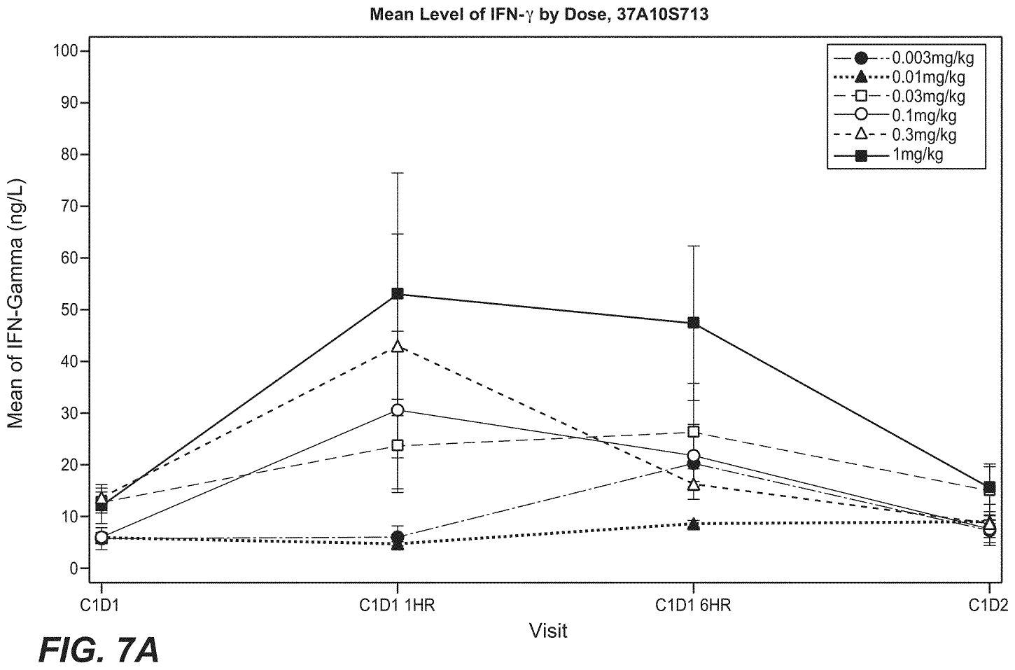

Using the data summarized above, FIG. 5 shows the PK profile and FIG. 6 shows the target engagement profile (TE) of 37A10S713 antibody in human subjects at the indicated doses.

Table 6A shows a summary of various pharmacokinetic parameters observed in the 37A10S713 monotherapy study and Table 6B shows a summary of various pharmacokinetic parameters observed in the 37A10S713+nivolumab combination therapy study.

TABLE 6A
Summary of 37A10S713 PK parameters* for 37A10S713 monotherapy

| | 37A10S713 Dose (mg/kg) | | | | | | |
|---|---|---|---|---|---|---|---|
| | 0.003 (n = 3) | 0.01 (n = 3) | 0.03 (n = 4) | 0.10 (n = 10) | 0.23 (n = 1)^{$} | 0.30 (n = 5) | 1.0 (n = 4) |
| AUC_{0-∞} | 1.75 (139%) | 7.90 (17%) | 45.6 (127%) | 309 (34%) | 911 | 1020 (78%) | 3110 (42%) |
| (μg · h/mL) | | | | | | | |
| AUC_{0-tz} | 1.50 (177%) | 7.68 (17%) | 44.2 (124%) | 293 (33%) | 784 | 592 (158%) | 2340 (35%) |
| (μg · h/mL) | | | | | | | |
| Cₘₐₓ (μg/mL) | 0.06 (61%) | 0.18 (15%) | 0.50 (57%) | 1.99 (28%) | 5.79 | 5.70 (22%) | 14.50 (21%) |
| CL (mL/h) | 1.71 (139%) | 1.27 (17%) | 0.66 (127%) | 0.32 (34%) | 0.25 | 0.29 (78%) | 0.32 (42%) |
| t1/2 (h)† | 30.1 (58%) | 30.8 (16%) | 83.4 (40%) | 106 (32%) | 196 | 227 (73%) | 175 (48%) |
| Vss (mL/kg) | 65.1 (46%) | 56.0 (30%) | 72.4 (65%) | 53.0 (28%) | 60.0 | 70.3 (44%) | 74.3 (29%) |

*Geometric mean (geometric CV %) was reported for all the parameters except for t1/2, mean, and (CV %) was reported.
^{$}One patient was accidentally dosed with this dose. This patient’s safety data was included with the 0.3 mg/kg dose group.

TABLE 6B
Summary of 37A10S713 PK parameters* for 37A10S173 +
nivolumab combination therapy
37A10S713 + Nivolumab

| | 37A10S713 Dose (mg/kg) | | | |
|---|---|---|---|---|
| | 0.010 (n = 3) | 0.030 (n = 2) | 0.10 (n = 3) | 0.30 (n = 3) |
| AUC_{0-∞} | 11 (56%) | 26 (95%) | 236 (44%) | 1070 (36%) |
| (μg · h/mL) | | | | |
| AUC_{0-tz} | 11 (56%) | 26 (95%) | 150 (160%) | 832 (21%) |
| (μg · h/mL) | | | | |
| Cₘₐₓ | 0.25 (26%) | 0.49 (19%) | 1.98 (19%) | 5.94 (12%) |
| (μg /mL) | | | | |
| CL (mL/h) | 0.90 (55%) | 1.15 (95%) | 0.42 (45%) | 0.28 (36%) |
| t1/2 (h)^{†} | 31.8 (22%) | 37.6 (86%) | 88.6 (43%) | 175 (46%) |
| Vss (mL/kg) | 41.6 (30%) | 51.2 (6%) | 52.5 (16%) | 64.3 (27%) |

*Geometric mean (geometric CV %) was reported for all the parameters except for t1/2, mean and (CV %) was reported.

In summary, target engagement was >90% through day 21 in 2 evaluable participants at 0.3 mg/kg 37A10S713 monotherapy, with no significant changes from baseline in CD4⁺ T cells, CD4⁺ T effector cells, CD4⁺ T regulatory cells, CD19⁺ B Cells, CD56⁺ NK Cells or CD8⁺ T cells. Anti-drug antibodies (ADA) to 37A10S713 were detected in 2/20 evaluable monotherapy participants and 3/10 evaluable combination therapy participants. ADA were transient except in one participant, where ADA was detected through 6 weeks. In one participant, PK appears to have been impacted.

The PK of 37A10S713 does not appear to be impacted by co-administration of nivolumab.

A dose of 0.3 mg/kg of 37A10S713 was selected for phase 2 monotherapy based on the safety and tolerability data observed in the phase 1 study, as well as the observed >90% target engagement through day 21, and the lack of peripheral T cell depletion. The PK was also found to be consistent with the preclinical model.

### Example 7: Identification of Genes Correlating with ICOS Expression

The level of ICOS across various tumor types was assessed at both the mRNA and the protein level. To specifically quantify ICOS levels across patient tumor samples, a novel RNA signature based approach was used. Analysis of high-dimensional gene expression was performed to define the immune component of tumors in an unbiased manner, using the high-dimensional data set from The Cancer Genome Atlas (TCGA) that include DNA mutational data, gene-expression data and gene-amplification data from 7,500 human tumors representing 24 different indications. Raw sequence data was prepared and gene expression values utilizing the fragments per kilobase of exon per million fragments mapped (FPKM) were computed by OmicSoft Corporation utilizing their proprietary data normalization and expression analysis pipeline. All the FPKM values were converted using a logarithmic function with a base 2 and analyzed. Before using the data for discovery of immune related signatures, the quality of the data was assessed using known biological patterns. For example, estrogen receptor expression was associated with the first principal component of the breast data, while microsatellite instability related signatures was associated with the third principal component of the colon data.

To identify a multigene signature to quantify the degree of ICOS expression in tumors, indications in which ICOS was overexpressed with a high coefficient of variance were first identified.

To identify a novel gene signature of ICOS expression, other genes tightly correlated with ICOS across the 13 major subtypes of cancer that had high and variable ICOS expression were examined. The spearman rank correlation (p) between a given gene and ICOS expression was calculated within each indication. The 300 genes most significantly associated with ICOS expression were retained for each indication, correlation p and nominal p-values retained for secondary analysis. All associations with ICOS were found to be significant at p<0.005. The average correlation rank was then computed across all indications for each gene, excluding values that had dropped during the first phase of analysis.

Genes selected to remain within the signature were those that were a) part of the top 300 genes associated with ICOS in at least 10 of the 13 indications tested and b) were, on average, within the top 75 genes associated with ICOS across indications in which they were part of the top 300 correlated genes. The genes identified using these metrics, along with the criteria for selection including the spearman correlation and the mean rank of correlation with ICOS are shown in Table 7. This analysis identified 39 genes, which can be used individually or in combination to predict the expression of ICOS across these 13 indications by RNA expression profiling. Table 7 shows the Spearman correlation coefficients (p) for 39 genes identified as correlating with ICOS expression. If a gene was not part of the top 300 genes correlated with ICOS expression no value is shown for the correlation coefficient. The average rank of correlation with ICOS is shown in the left most column across all indications in which the gene was part of the top 300 genes correlated with ICOS. Genes were selected that were found to be within the top 300 genes associated with ICOS expression in at least 10 of the indications shown above. ICOS signature genes also were required to have a mean rank within the top 75 genes of any indication in which why were in the top 300 genes. Indication abbreviations are: Bladder Cancer (BLCA), triple negative breast cancer (BRCA TN), cervical cancer (CESC), microsatellite stable colorectal cancer (CO MSS), head & neck cancer (HNSC), clear cell kidney cancer (KIRC) lung adenocarcinoma (LUAD, a sub-type of non-small cell lung cancer (NSCLC)), lung squamous cell carcinoma (LUSC, a sub-type of non-small cell lung cancer (NSCLC)), ovarian cancer (OV), pancreatic cancer (PAAD), melanoma (SKCM), stomach cancer (STAD).

TABLE 7
mRNAs correlated with ICOS expression

| | Exemplary mRNA | | BRCA | | CO | HNSC | HNSC | | | | | | | | Mean |
|---|---|---|---|---|---|---|---|---|---|---|---|---|---|---|---|
| Gene | Acc. No. | BLCA | TN | CESC | MSS | HPV− | HPV+ | KIRC | LUAD | LUSC | OV | PAAD | SKCM | STAD | Rank |
| CCR5 | NM_000579.3 | 0.876 | 0.901 | 0.887 | | 0.847 | 0.789 | 0.880 | 0.817 | 0.866 | 0.834 | 0.799 | 0.841 | 0.820 | 23 |
| CD2 | NM_001767.3 | 0.905 | 0.941 | 0.858 | 0.680 | 0.858 | 0.780 | 0.889 | 0.881 | 0.903 | 0.896 | 0.849 | 0.881 | 0.841 | 26 |
| CD3D | NM_000732.4 | 0.822 | 0.891 | 0.805 | 0.738 | 0.827 | 0.792 | 0.852 | 0.833 | 0.878 | 0.836 | 0.848 | 0.860 | 0.759 | 40 |
| CD3E | NM_000733.3 | 0.896 | 0.903 | 0.849 | | 0.841 | 0.795 | 0.853 | 0.847 | 0.883 | 0.855 | 0.837 | 0.839 | 0.750 | 26 |
| CD3G | NM_000073.2 | 0.833 | 0.912 | 0.795 | 0.765 | 0.833 | 0.890 | 0.855 | 0.812 | 0.839 | 0.795 | 0.783 | 0.879 | 0.822 | 33 |
| CD48 | NM_001256030.1 | 0.833 | 0.882 | 0.877 | 0.749 | 0.794 | 0.764 | 0.742 | 0.775 | 0.834 | 0.781 | 0.730 | 0.826 | 0.743 | 71 |
| CD5 | NM_014207.3 | 0.851 | 0.889 | 0.849 | | 0.788 | 0.820 | 0.803 | 0.771 | 0.832 | 0.830 | 0.807 | 0.830 | | 45 |
| CD96 | NM_198196.2 | | 0.880 | 0.775 | 0.690 | 0.774 | 0.765 | 0.872 | 0.803 | 0.829 | 0.797 | 0.815 | 0.797 | 0.843 | 73 |
| CTLA4 | NM_005214.4 | 0.942 | 0.925 | 0.892 | | 0.944 | 0.853 | 0.825 | 0.860 | 0.910 | 0.889 | 0.876 | | 0.804 | 10 |
| CXCR6 | NM_006564.1 | 0.862 | 0.904 | 0.790 | 0.741 | 0.853 | 0.900 | 0.845 | 0.830 | 0.869 | 0.872 | 0.798 | 0.853 | 0.824 | 27 |
| FOXP3 | NM_014009.3 | 0.905 | 0.886 | 0.816 | | 0.886 | 0.845 | 0.715 | 0.778 | 0.858 | 0.812 | 0.749 | | 0.763 | 50 |
| ICOS | NM_012092.3 | 1.000 | 1.000 | 1.000 | 1.000 | 1.000 | 1.000 | 1.000 | 1.000 | 1.000 | 1.000 | 1.000 | 1.000 | 1.000 | 1 |
| IKZF1 | NM_006060.5 | 0.821 | 0.869 | 0.799 | 0.752 | 0.807 | 0.901 | 0.829 | 0.782 | 0.808 | 0.690 | 0.752 | 0.841 | 0.792 | 67 |
| IL21R | NM_181078.2 | 0.898 | 0.868 | 0.912 | 0.711 | 0.874 | 0.833 | 0.687 | 0.787 | 0.874 | 0.838 | | 0.817 | 0.764 | 53 |
| IL2RB | NM_000878.3 | 0.885 | 0.894 | 0.729 | | 0.822 | 0.862 | 0.788 | 0.791 | 0.819 | 0.827 | 0.803 | 0.826 | 0.829 | 51 |
| ITGAL | NM_002209.2 | 0.583 | 0.894 | 0.867 | | 0.834 | 0.848 | 0.801 | 0.785 | 0.830 | 0.794 | 0.692 | 0.834 | 0.769 | 50 |
| ITK | NM_005546.3 | 0.893 | 0.916 | 0.770 | 0.767 | 0.828 | 0.804 | 0.850 | 0.843 | 0.876 | 0.881 | 0.832 | 0.808 | 0.811 | 35 |
| KIAA0748 | NM_001136030.2 | | 0.892 | 0.849 | | 0.816 | 0.783 | 0.783 | 0.792 | 0.825 | 0.786 | 0.755 | 0.828 | 0.707 | 62 |
| LCP2 | NM_005565.3 | 0.862 | 0.877 | 0.841 | | 0.838 | 0.833 | 0.766 | 0.810 | 0.854 | 0.795 | 0.728 | | 0.828 | 44 |
| LTA | NM_001159740.2 | 0.816 | 0.901 | 0.843 | 0.741 | 0.796 | 0.763 | 0.837 | 0.810 | 0.819 | 0.808 | 0.775 | 0.805 | 0.695 | 69 |
| P2RY10 | NM_014499.2 | 0.889 | 0.902 | 0.826 | 0.780 | 0.806 | 0.802 | 0.874 | 0.804 | 0.867 | 0.859 | 0.835 | 0.865 | 0.823 | 24 |
| PTPRC | NM_002838.4 | 0.904 | 0.880 | 0.829 | 0.764 | 0.836 | 0.872 | 0.784 | 0.820 | 0.867 | 0.798 | 0.740 | 0.840 | 0.838 | 34 |
| PYHIN1 | NM_152501.4 | 0.850 | 0.906 | 0.807 | | 0.770 | 0.758 | 0.839 | 0.836 | 0.845 | 0.810 | 0.844 | 0.814 | 0.818 | 52 |
| SASH3 | NM_018990.3 | 0.876 | 0.880 | 0.881 | 0.674 | 0.850 | 0.863 | 0.803 | 0.815 | 0.866 | 0.755 | 0.712 | 0.795 | 0.735 | 66 |
| SH2D1A | NM_002351.4 | 0.884 | 0.907 | 0.898 | 0.681 | 0.805 | 0.900 | 0.860 | 0.881 | 0.868 | 0.848 | 0.843 | 0.859 | 0.795 | 28 |
| SIRPG | NM_018556.3 | 0.760 | 0.891 | 0.831 | | 0.842 | 0.796 | 0.872 | 0.848 | 0.862 | 0.814 | 0.848 | 0.847 | 0.780 | 43 |
| SIT1 | NM_014450.2 | 0.824 | 0.852 | 0.851 | 0.761 | 0.781 | 0.796 | 0.848 | 0.794 | 0.811 | 0.822 | 0.794 | 0.838 | 0.636 | 73 |
| SLA2 | NM_032214.3 | 0.894 | 0.900 | 0.751 | | 0.837 | 0.796 | 0.867 | 0.804 | 0.864 | 0.839 | 0.762 | 0.873 | 0.797 | 39 |
| SLAMF1 | NM_003037.3 | 0.901 | 0.897 | 0.888 | 0.684 | 0.807 | 0.798 | 0.764 | 0.817 | 0.864 | 0.880 | 0.885 | 0.770 | 0.799 | 54 |
| SLAMF6 | NM_001184714.1 | 0.843 | 0.891 | 0.791 | | 0.760 | 0.896 | 0.847 | 0.769 | 0.836 | 0.855 | 0.761 | 0.862 | 0.759 | 53 |
| SNX20 | NM_182854.2 | 0.796 | 0.888 | 0.814 | 0.738 | 0.817 | 0.770 | 0.794 | 0.773 | 0.849 | 0.764 | 0.700 | 0.838 | 0.747 | 75 |
| SP140 | NM_007237.4 | 0.801 | 0.912 | 0.851 | | | 0.761 | 0.838 | 0.755 | 0.818 | 0.771 | 0.789 | 0.843 | 0.693 | 73 |
| TCR-α | NG_001332.2* | 0.828 | 0.904 | 0.731 | 0.762 | 0.821 | 0.805 | 0.788 | 0.835 | 0.848 | 0.816 | 0.827 | 0.837 | 0.802 | 51 |
| TCRVB | NG_001333.2* | | 0.872 | 0.848 | 0.701 | 0.812 | 0.810 | 0.826 | 0.832 | 0.884 | 0.853 | | 0.840 | 0.745 | 47 |
| TIGIT | NM_173799.3 | 0.888 | 0.920 | 0.872 | 0.738 | 0.861 | 0.827 | 0.906 | 0.868 | 0.849 | 0.888 | 0.918 | 0.893 | 0.830 | 14 |
| TRA | NG_001332.2* | 0.841 | 0.911 | 0.765 | 0.666 | 0.810 | 0.778 | 0.793 | 0.805 | 0.839 | 0.807 | 0.762 | 0.843 | 0.772 | 70 |
| TRAC | NG_001332.2* | 0.891 | 0.909 | 0.850 | 0.684 | 0.853 | 0.791 | 0.845 | 0.868 | 0.877 | 0.849 | 0.841 | 0.840 | 0.794 | 33 |
| TRAT1 | NM_016388.2 | 0.802 | 0.917 | 0.810 | 0.770 | 0.737 | 0.781 | 0.882 | 0.851 | 0.839 | 0.851 | 0.842 | 0.867 | 0.804 | 48 |
| UBASH3A | NM_018961.3 | 0.849 | 0.902 | 0.766 | | 0.812 | 0.758 | 0.872 | 0.789 | 0.839 | 0.758 | 0.715 | 0.855 | 0.771 | 67 |
| Average ρ | | 0.863 | 0.899 | 0.832 | 0.739 | 0.828 | 0.820 | 0.830 | 0.820 | 0.856 | 0.827 | 0.801 | 0.844 | 0.787 | |

*Accessions for genomic loci provided for TCR genes.

The list of mRNAs in Table 7 may be used to form a panel of mRNAs for determining the expression level of ICOS, e.g., to create a more robust assay than an assay that detects ICOS alone. In some embodiments, a panel is formed from the set of mRNAs: CCR5, CD2, CD96, CTLA4, CXCR6, FOXP3, ICOS, ITK, P2RY10, SIRPG, and TIGIT.

The disclosure may be embodied in other specific forms without departing from the spirit or essential characteristics thereof. The foregoing embodiments are therefore to be considered in all respects illustrative rather than limiting of the disclosure. Scope of the disclosure is thus indicated by the appended claims rather than by the foregoing description, and all changes that come within the meaning and range of equivalency of the claims are therefore intended to be embraced herein.

Table of Sequences

| SEQ | | |
|---|---|---|
| ID | | |
| NO | Description | Sequence |
| 1 | Human ICOS precursor | MKSGLWYFFL FCLRIKVLTG EINGSANYEM FIFHNGGVQI |
| | (with signal sequence); | LCKYPDIVQQ FKMQLLKGGQ ILCDLTKTKG SGNTVSIKSL |
| | UniProtKB/Swiss-Prot: | KFCHSQLSNN SVSFFLYNLD HSHANYYFCN LSIFDPPPFK |
| | Q9Y6W8.1; 7 Jan. 2015 | VTLTGGYLHI YESQLCCQLK FWLPIGCAAF VVVCILGCIL |
| | | ICWLTKKKYS SSVHDPNGEY MFMRAVNTAK KSRLTDVTL |
| 2 | Human mature ICOS | EINGSANYEM FIFHNGGVQI LCKYPDIVQQ FKMQLLKGGQ |
| | (without signal sequence) | ILCDLTKTKG SGNTVSIKSL KFCHSQLSNN SVSFFLYNLD |
| | | HSHANYYFCN LSIFDPPPFK VTLTGGYLHI YESQLCCQLK |
| | | FWLPIGCAAF VVVCILGCIL ICWLTKKKYS SSVHDPNGEY |
| | | MFMRAVNTAK KSRLTDVTL |
| 3 | Mouse ICOS precursor | MKPYFCRVFV FCFLIRLLTG EINGSADHRM FSFHNGGVQI |
| | (with signal sequence); | SCKYPETVQQ LKMRLFRERE VLCELTKTKG SGNAVSIKNP |
| | UniProtKB/Swiss-Prot: | MLCLYHLSNN SVSFFLNNPD SSQGSYYFCS LSIFDPPPFQ |
| | Q9WVS0.2; 7 Jan. 2015 | ERNLSGGYLH IYESQLCCQL KLWLPVGCAA FVVVLLFGCT |
| | | LIIWFSKKKY GSSVHDPNSE YMFMAAVNTN KKSRLAGVTS |
| 4 | Mouse mature ICOS | EINGSADHRM FSFHNGGVQI SCKYPETVQQ LKMRLFRERE |
| | (without signal sequence) | VLCELTKTKG SGNAVSIKNP MLCLYHLSNN SVSFFLNNPD |
| | | SSQGSYYFCS LSIFDPPPFQ ERNLSGGYLH IYESQLCCQL |
| | | KLWLPVGCAA FVVVLLFGCT LIIWFSKKKY GSSVHDPNSE |
| | | YMFMAAVNTN KKSRLAGVTS |
| 5 | Cynomolgus monkey ICOS | MKSGLWYFFL FCLHMKVLTG EINGSANYEM FIFHNGGVQI |
| | precursor (with signal | LCKYPDIVQQ FKMQLLKGGQ ILCDLTKTKG SGNKVSIKSL |
| | sequence) | KFCHSQLSNN SVSFFLYNLD RSHANYYFCN LSIFDPPPFK |
| | | VTLTGGYLHI YESQLCCQLK FWLPIGCATF VVVCIFGCIL |
| | | ICWLTKKKYS STVHDPNGEY MFMRAVNTAK KSRLTGTTP |
| 6 | Cynomolgus mature ICOS | EINGSANYEM FIFHNGGVQI LCKYPDIVQQ FKMQLLKGGQ |
| | (without signal sequence) | ILCDLTKTKG SGNKVSIKSL KFCHSQLSNN SVSFFLYNLD |
| | | RSHANYYFCN LSIFDPPPFK VTLTGGYLHI YESQLCCQLK |
| | | FWLPIGCATF VVVCIFGCIL ICWLTKKKYS STVHDPNGEY |
| | | MFMRAVNTAK KSRLTGTTP |
| 20 | 37A10 heavy chain variable | EVQLVESGGG LVKPGGSLKL SCAASGFTFS DYWMDWVRQA |
| | region | PGKGLEWVGN IDEDGSITEY SPFVKGRFTI SRDNVKNTLY |
| | | LQMNSVKSED TATYYCTRWG RFGFDSWGQG TLVTVSS |
| 21 | 37A10 light chain variable | DIVMTQSPSS LAVSAGDRVT INCKSSQSLL SGSFNYLTWY |
| | region | QQKTGQAPKL LIFYASTRHT GVPDRFMGSG SGTDFTLTIN |
| | | SFQTEDLGDY YCHHHYNAPP TFGPGTKLEL R |
| 22 | 37A10 VH CDR1 | GFTFSDYWMD |
| 23 | 37A10 VH CDR2 | NIDEDGSITEYSPFVKG |
| 24 | 37A10 VH CDR3 | WGRFGFDS |
| 25 | 37A10 VL CDR1 | KSSQSLLSGSFNYLT |
| 26 | 37A10 VL CDR2 | YASTRHT |
| 27 | 37A10 VL CDR3 | HHHYNAPPT |
| 60 | 37A10S713 heavy chain | EVQLVESGGG LVQPGGSLRL SCAASGFTFS DYWMDWVRQA |
| | variable region | PGKGLVWVSN IDEDGSITEY SPFVKGRFTI SRDNAKNTLY |
| | | LQMNSLRAED TAVYYCTRWG RFGFDSWGQG TLVTVSS |
| 61 | 37A10S713 light chain | DIVMTQSPDS LAVSLGERAT INCKSSQSLL SGSFNYLTWY |
| | variable region | QQKPGQPPKL LIFYASTRHT GVPDRFSGSG SGTDFTLTIS |
| | | SLQAEDVAVY YCHHHYNAPP TFGPGTKVDI K |
| 62 | 37A10S713 VH CDR1 | GFTFSDYWMD |
| 63 | 37A10S713 VH CDR2 | NIDEDGSITEYSPFVKG |
| 64 | 37A10S713 VH CDR3 | WGRFGFDS |
| 65 | 37A10S713 VL CDR1 | KSSQSLLSGSFNYLT |
| 66 | 37A10S713 VL CDR2 | YASTRHT |
| 67 | 37A10S713 VL CDR3 | HHHYNAPPT |
| 70 | 37A10S714 heavy chain | EVQLVESGGG LVQPGGSLRL SCAASGFTFS DYWMDWVRQA |
| | variable region | PGKGLVWVSN IDEDGSITEY SPFVKGRFTI SRDNAKNTLY |
| | | LQMNSLRAED TAVYYCTRWG RFGFDSWGQG TLVTVSS |
| 71 | 37A10S714 light chain | DIVMTQSPDS LAVSLGERAT INCKSSQSLL SGSFNYLTWY |
| | variable region | QQKPGQPPKL LIFYASTRET GVPDRFSGSG SGTDFTLTIS |
| | | SLQAEDVAVY YCHHHYNAPP TFGPGTKVDI K |
| 72 | 37A10S714 VH CDR1 | GFTFSDYWMD |
| 73 | 37A10S714 VH CDR2 | NIDEDGSITEYSPFVKG |
| 74 | 37A10S714 VH CDR3 | WGRFGFDS |
| 75 | 37A10S714 VL CDR1 | KSSQSLLSGSFNYLT |
| 76 | 37A10S714 VL CDR2 | YASTRET |
| 77 | 37A10S714 VL CDR3 | HHHYNAPPT |
| 80 | 37A10S715 heavy chain | EVQLVESGGG LVQPGGSLRL SCAASGFTFS DYWMDWVRQA |
| | variable region | PGKGLVWVSN IDEDGSITEY SPFVKGRFTI SRDNAKNTLY |
| | | LQMNSLRAED TAVYYCTRWG RFGFDSWGQG TLVTVSS |
| 81 | 37A10S715 light chain | DIVMTQSPDS LAVSLGERAT INCKSSQSLL SGSFNYLTWY |
| | variable region | QQKPGQPPKL LIFYASTRQT GVPDRFSGSG SGTDFTLTIS |
| | | SLQAEDVAVY YCHHHYNAPP TFGPGTKVDI K |
| 82 | 37A10S715 VH CDR1 | GFTFSDYWMD |
| 83 | 37A10S715 VH CDR2 | NIDEDGSITEYSPFVKG |
| 84 | 37A10S715 VH CDR3 | WGRFGFDS |
| 85 | 37A10S715 VL CDR1 | KSSQSLLSGSFNYLT |
| 86 | 37A10S715 VL CDR2 | YASTRQT |
| 87 | 37A10S715 VL CDR3 | HHHYNAPPT |
| 90 | 37A10S716 heavy chain | EVQLVESGGG LVQPGGSLRL SCAASGFTFS DYWMDWVRQA |
| | variable region | PGKGLVWVSN IDESGSITEY SPFVKGRFTI SRDNAKNTLY |
| | | LQMNSLRAED TAVYYCTRWG RFGFDSWGQG TLVTVSS |
| 91 | 37A10S716 light chain | DIVMTQSPDS LAVSLGERAT INCKSSQSLL SGSFNYLTWY |
| | variable region | QQKPGQPPKL LIFYASTRHT GVPDRFSGSG SGTDFTLTIS |
| | | SLQAEDVAVY YCHHHYNAPP TFGPGTKVDI K |
| 92 | 37A10S716 VH CDR1 | GFTFSDYWMD |
| 93 | 37A10S716 VH CDR2 | NIDESGSITEYSPFVKG |
| 94 | 37A10S716 VH CDR3 | WGRFGFDS |
| 95 | 37A10S716 VL CDR1 | KSSQSLLSGSFNYLT |
| 96 | 37A10S716 VL CDR2 | YASTRHT |
| 97 | 37A10S716 VL CDR3 | HHHYNAPPT |
| 100 | 37A10S717 heavy chain | EVQLVESGGG LVQPGGSLRL SCAASGFTFS DYWMDWVRQA |
| | variable region | PGKGLVWVSN IDESGSITEY SPFVKGRFTI SRDNAKNTLY |
| | | LQMNSLRAED TAVYYCTRWG RFGFDSWGQG TLVTVSS |
| 101 | 37A10S717 light chain | DIVMTQSPDS LAVSLGERAT INCKSSQSLL SGSFNYLTWY |
| | variable region | QQKPGQPPKL LIFYASTRET GVPDRFSGSG SGTDFTLTIS |
| | | SLQAEDVAVY YCHHHYNAPP TFGPGTKVDI K |
| 102 | 37A10S717 VH CDR1 | GFTFSDYWMD |
| 103 | 37A10S717 VH CDR2 | NIDESGSITEYSPFVKG |
| 104 | 37A10S717 VH CDR3 | WGRFGFDS |
| 105 | 37A10S717 VL CDR1 | KSSQSLLSGSFNYLT |
| 106 | 37A10S717 VL CDR2 | YASTRET |
| 107 | 37A10S717 VL CDR3 | HHHYNAPPT |
| 110 | 37A10S718 heavy chain | EVQLVESGGG LVQPGGSLRL SCAASGFTFS DYWMDWVRQA |
| | variable region | PGKGLVWVSN IDESGSITEY SPFVKGRFTI SRDNAKNTLY |
| | | LQMNSLRAED TAVYYCTRWG RFGFDSWGQG TLVTVSS |
| 111 | 37A10S718 light chain | DIVMTQSPDS LAVSLGERAT INCKSSQSLL SGSFNYLTWY |
| | variable region | QQKPGQPPKL LIFYASTRQT GVPDRFSGSG SGTDFTLTIS |
| | | SLQAEDVAVY YCHHHYNAPP TFGPGTKVDI K |
| 112 | 37A10S718 VH CDR1 | GFTFSDYWMD |
| 113 | 37A10S718 VH CDR2 | NIDESGSITEYSPFVKG |
| 114 | 37A10S718 VH CDR3 | WGRFGFDS |
| 115 | 37A10S718 VL CDR1 | KSSQSLLSGSFNYLT |
| 116 | 37A10S718 VL CDR2 | YASTRQT |
| 117 | 37A10S718 VL CDR3 | HHHYNAPPT |
| 188 | 37A10S713 human IgG1 | EVQLVESGGG LVQPGGSLRL SCAASGFTFS DYWMDWVRQA |
| | heavy chain | PGKGLVWVSN IDEDGSITEY SPFVKGRFTI SRDNAKNTLY |
| | | LQMNSLRAED TAVYYCTRWG RFGFDSWGQG TLVTVSSAST |
| | | KGPSVFPLAP SSKSTSGGTA ALGCLVKDYF PEPVTVSWNS |
| | | GALTSGVHTF PAVLQSSGLY SLSSVVTVPS SSLGTQTYIC |
| | | NVNHKPSNTK VDKKVEPKSC DKTHTCPPCP APELLGGPSV |
| | | FLFPPKPKDT LMISRTPEVT CVVVDVSHED PEVKFNWYVD |
| | | GVEVHNAKTK PREEQYNSTY RVVSVLTVLH QDWLNGKEYK |
| | | CKVSNKALPA PIEKTISKAK GQPREPQVYT LPPSREEMTK |
| | | NQVSLTCLVK GFYPSDIAVE WESNGQPENN YKTTPPVLDS |
| | | DGSFFLYSKL TVDKSRWQQG NVFSCSVMHE ALHNHYTQKS |
| | | LSLSPGK |
| 189 | 37A10S713 human κ light | DIVMTQSPDS LAVSLGERAT INCKSSQSLL SGSFNYLTWY |
| | chain | QQKPGQPPKL LIFYASTRHT GVPDRFSGSG SGTDFTLTIS |
| | | SLQAEDVAVY YCHHHYNAPP TFGPGTKVDI KRTVAAPSVF |
| | | IFPPSDEQLK SGTASVVCLL NNFYPREAKV QWKVDNALQS |
| | | GNSQESVTEQ DSKDSTYSLS STLTLSKADY EKHKVYACEV |
| | | THQGLSSPVT KSFNRGEC |
| 190 | Rat ICOS precursor (with | MKPYFSCVFV FCFLIKLLTG ELNDLANHRM FSFHDGGVQI |
| | signal sequence); UniProt | SCNYPETVQQ LKMQLFKDRE VLCDLTKTKG SGNTVSIKNP |
| | Q9R1T7 | MSCPYQLSNN SVSFFLDNAD SSQGSYFLCS LSIFDPPPFQ |
| | | EKNLSGGYLL IYESQLCCQL KLWLPVGCAA FVAALLFGCI |
| | | FIVWFAKKKY RSSVHDPNSE YMFMAAVNTN KKSRLAGMTS |
| 191 | Mature rat ICOS (without | ELNDLANHRM FSFHDGGVQI SCNYPETVQQ LKMQLFKDRE |
| | signal sequence) | VLCDLTKTKG SGNTVSIKNP MSCPYQLSNN SVSFFLDNAD |
| | | SSQGSYFLCS LSIFDPPPFQ EKNLSGGYLL IYESQLCCQL |
| | | KLWLPVGCAA FVAALLFGCI FIVWFAKKKY RSSVHDPNSE |
| | | YMFMAAVNTN KKSRLAGMTS |
| 192 | 2M13 heavy chain variable | EVQLQQSGAE LVRPGAVVKL SCKASGFDIK DYYMHWVQQR |
| | region | PEQGLEWIGW IDPENGNAVY DPQFQGKASI TADTSSNTAY |
| | | LQLSSLTSED TAVYYCASDY YGSKGYLDVW GAGTTVTVSS |
| 193 | 2M13 light chain variable | QIVLTQSPTI MSASPGEKVT ITCSASSSVS YMHWFQQKPG |
| | region | TSPKLWIYST SNLASGVPAR FGGSRSGTSY SLTISRMEAE |
| | | DAATYYCQQR SSYPFTFGSG TKLEIK |
| 194 | 2M13 VH CDR1 | DYYMH |
| 195 | 2M13 VH CDR2 | WIDPENGNAVYDPQFQG |
| 196 | 2M13 VH CDR3 | DYYGSKGYLDV |
| 197 | 2M13 VL CDR1 | SASSSVSYMH |
| 198 | 2M13 VL CDR2 | STSNLAS |
| 199 | 2M13 VL CDR3 | QQRSSYPFT |
| 200 | 2M19 heavy chain variable | EVQLQQSGAE LVRSGASVKL SCTTSAFNII DYYMHWVIQR |
| | region | PEQGLEWIAW IDPENGDPEY APKFQDKATM TTDTSSNTAY |
| | | LQLSSLTSED TAVYYCTAWR GFAYWGQGTL VTVSA |
| 201 | 2M19 light chain variable | DVVMTQTPLS LPVSLGDQAS ISCRSSQSLV HSNGNTYLHW |
| | region | YLQKPGQSPK LLIYKVSNRF SGVPDRFSGS GSGTDFTLKI |
| | | SRVEAEDLGV YFCSQSIHVP PTFGGGTKLE IK |
| 202 | 2M19 VH CDR1 | DYYMH |
| 203 | 2M19 VH CDR2 | WIDPENGDPEYAPKFQD |
| 204 | 2M19 VH CDR3 | WRGFAY |
| 205 | 2M19 VL CDR1 | RSSQSLVHSNGNTYLH |
| 206 | 2M19 VL CDR2 | KVSNRFS |
| 207 | 2M19 VL CDR3 | SQSIHVPPT |
| 208 | 2M24 heavy chain variable | EVQLQQSGAE LVRSGASVKL SCTASGFNIR DYYMHWVRQR |
| | region | PEQGLEWIGW IDPENGDIDY APKFQDKATM TADTSSNTAY |
| | | LQLSSLTSED SAVYYFTAWK GLAYWGQGTL VTVSA |
| 209 | 2M24 light chain variable | DVVMTQTPLS LPVSLGDQAS MSCRSSQSLV HSNGNTYLQW |
| | region | YLQKPGQSPK LLIYKVFNRF SGVPDRFSGS GSGTDFTLKI |
| | | SRVEAEDLGV YFCSQSTHVP PTFGGGTKLE IK |
| 210 | 2M24 VH CDR1 | DYYMH |
| 211 | 2M24 VH CDR2 | WIDPENGDIDYAPKFQD |
| 212 | 2M24 VH CDR3 | WKGLAY |
| 213 | 2M24 VL CDR1 | RSSQSLVHSNGNTYLQ |
| 214 | 2M24 VL CDR2 | KVFNRFS |
| 215 | 2M24 VL CDR3 | SQSTHVPPT |
| 216 | 37A10S713 human IgG1 | EVQLVESGGG LVQPGGSLRL SCAASGFTFS DYWMDWVRQA |
| | heavy chain V2 | PGKGLVWVSN IDEDGSITEY SPFVKGRFTI SRDNAKNTLY |
| | | LQMNSLRAED TAVYYCTRWG RFGFDSWGQG TLVTVSSAST |
| | | KGPSVFPLAP SSKSTSGGTA ALGCLVKDYF PEPVTVSWNS |
| | | GALTSGVHTF PAVLQSSGLY SLSSVVTVPS SSLGTQTYIC |
| | | NVNHKPSNTK VDKKVEPKSC DKTHTCPPCP APELLGGPSV |
| | | FLFPPKPKDT LMISRTPEVT CVVVDVSHED PEVKFNWYVD |
| | | GVEVHNAKTK PREEQYNSTY RVVSVLTVLH QDWLNGKEYK |
| | | CKVSNKALPA PIEKTISKAK GQPREPQVYT LPPSREEMTK |
| | | NQVSLTCLVK GFYPSDIAVE WESNGQPENN YKTTPPVLDS |
| | | DGSFFLYSKL TVDKSRWQQG NVFSCSVMHE ALHNHYTQKS |
| | | LSLSPG |

1. A method of treating cancer in a subject, comprising administering a dose of 0.3 mg/kg of an anti-ICOS antibody to said subject, wherein said anti-ICOS antibody comprises an HCDR1 comprising the amino acid sequence of SEQ ID NO: 62; an HCDR2 comprising the amino acid sequence of SEQ ID NO: 63; an HCDR3 comprising the amino acid sequence of SEQ ID NO: 64; an LCDR1 comprising the amino acid sequence of SEQ ID NO: 65; an LCDR2 comprising the amino acid sequence of SEQ ID NO: 66; and an LCDR3 comprising the amino acid sequence of SEQ ID NO: 67.

2. The method of claim 1, wherein said dose is administered once every three weeks.

3. The method of claim 1, wherein said dose is administered once every four weeks.

4. The method of claim 1, wherein said dose is administered once every six weeks.

5. A method of treating cancer in a subject, comprising administering a dose of 0.1 mg/kg of an anti-ICOS antibody to said subject, wherein said anti-ICOS antibody comprises an HCDR1 comprising the amino acid sequence of SEQ ID NO: 62; an HCDR2 comprising the amino acid sequence of SEQ ID NO: 63; an HCDR3 comprising the amino acid sequence of SEQ ID NO: 64; an LCDR1 comprising the amino acid sequence of SEQ ID NO: 65; an LCDR2 comprising the amino acid sequence of SEQ ID NO: 66; and an LCDR3 comprising the amino acid sequence of SEQ ID NO: 67.

6. The method of claim 5, wherein said dose is administered once every three weeks.

7. The method of claim 5, wherein said dose is administered once every four weeks.

8. The method of claim 5, wherein said dose is administered once every six weeks.

9. (canceled)

10. The method of claim 1, wherein, prior to said administering, said method further comprises selecting said subject for treatment with said anti-ICOS antibody, wherein said selecting comprises:
a) detecting the levels of at least two, at least three, at least four, at least five, at least six, at least seven, at least eight, at least nine, or at least ten mRNAs selected from the mRNAs in Table 7 in a sample from a subject; and
b) if the level of at least one, at least two, at least three, at least four, at least five, at least six, at least seven, at least eight, at least nine, or at least ten of the mRNAs is above a threshold level, then selecting said subject for treatment with said anti-ICOS antibody.

11. (canceled)

12. (canceled)

13. The method of claim 10, wherein the method comprises detecting the levels of at least two, at least three, at least four, at least five, at least six, at least seven, at least eight, at least nine, or at least ten mRNAs selected from CCR5, CD2, CD96, CTLA4, CXCR6, FOXP3, ICOS, ITK, P2RY10, SIRPG, and TIGIT.

14. The method of claim 13, wherein the detecting comprises at least one method selected from amplification and hybridization.

15. (canceled)

16. (canceled)

17. The method of claim 10, wherein the sample is a cancer sample.

18. The method of claim 1, wherein, prior to said administering, said method further comprises selecting said subject for treatment with said anti-ICOS antibody, wherein said selecting comprises contacting T cells from said subject with a test agonist anti-ICOS antibody and determining whether NKp46 ligand (NKp46-L) is induced on the T cells wherein if NKp46-L is induced on the T cells, the subject is selected for treatment with said anti-ICOS agonist antibody.

19. The method of claim 1, wherein, prior to said administering, said method further comprises selecting said subject for treatment with said anti-ICOS antibody, wherein said selecting comprises detecting the level of ICOS in a sample from the subject.

20. (canceled)

21. The method of claim 19, wherein the detecting comprises immunohistochemistry, wherein the immunohistochemistry comprises contacting the sample with an antibody selected from:
(i) an antibody comprising (a) HCDR1 comprising the amino acid sequence of SEQ ID NO: 194; (b) HCDR2 comprising the amino acid sequence of SEQ ID NO: 195; (c) HCDR3 comprising the amino acid sequence of SEQ ID NO: 196; (d) LCDR1 comprising the amino acid sequence of SEQ ID NO: 197; (e) LCDR2 comprising the amino acid sequence of SEQ ID NO: 198; and (f) LCDR3 comprising the amino acid sequence of SEQ ID NO: 199; or
(ii) an antibody comprising (a) HCDR1 comprising the amino acid sequence of SEQ ID NO: 202; (b) HCDR2 comprising the amino acid sequence of SEQ ID NO: 203; (c) HCDR3 comprising the amino acid sequence of SEQ ID NO: 204; (d) LCDR1 comprising the amino acid sequence of SEQ ID NO: 205; (e) LCDR2 comprising the amino acid sequence of SEQ ID NO: 206; and (f) LCDR3 comprising the amino acid sequence of SEQ ID NO: 207; or
(iii) an antibody comprising (a) HCDR1 comprising the amino acid sequence of SEQ ID NO: 210; (b) HCDR2 comprising the amino acid sequence of SEQ ID NO: 211; (c) HCDR3 comprising the amino acid sequence of SEQ ID NO: 212; (d) LCDR1 comprising the amino acid sequence of SEQ ID NO: 213; (e) LCDR2 comprising the amino acid sequence of SEQ ID NO: 214; and (f) LCDR3 comprising the amino acid sequence of SEQ ID NO: 215.

22. (canceled)

23. (canceled)

24. (canceled)

25. The method of claim 1, wherein the subject has a cancer selected from melanoma, non-small cell lung cancer (NSCLC), renal cell carcinoma (RCC) (e.g., clear cell RCC), gastric cancer, bladder cancer, endometrial cancer, MSI-H cancer of any organ, diffuse large B-cell lymphoma (DLBCL), Hodgkin's lymphoma, ovarian cancer (e.g., endometrioid ovarian cancer), head & neck squamous cell cancer (HNSCC), acute myeloid leukemia (AML), rectal cancer, refractory testicular cancer, small cell lung cancer (SCLC), small bowel cancer, metastatic cutaneous squamous cell cancer, cervical cancer, MSI-high colon cancer, esophageal cancer, mesothelioma, breast cancer, and triple negative breast cancer (TNBC).

26. (canceled)

27. (canceled)

28. (canceled)

29. (canceled)

30. The method of claim 1, wherein the anti-ICOS antibody comprises a heavy chain variable region (VH) and a light chain variable region (VL), wherein the VH is at least 90%, 91%, 92%, 93%, 94%, 95%, 96%, 97%, 98%, 99%, or 100% identical to the amino acid sequence of SEQ ID NO: 60 and the VL is at least 90%, 91%, 92%, 93%, 94%, 95%, 96%, 97%, 98%, 99%, or 100% identical to the amino acid sequence of SEQ ID NO: 61.

31. The method of claim 30, wherein said VH comprises the amino acid sequence of SEQ ID NO: 60 and said VL comprises the amino acid sequence of SEQ ID NO: 61.

32. (canceled)

33. (canceled)

34. (canceled)

35. The method of claim 1, wherein the anti-ICOS antibody comprises a heavy chain comprising the amino acid sequence of SEQ ID NO: 188 and a light chain comprising the amino acid sequence of SEQ ID NO: 189.

36. The method of claim 29, wherein the anti-ICOS antibody consists of a heavy chain having the amino acid sequence of SEQ ID NO: 188 and a light chain having the amino acid sequence of SEQ ID NO: 189.

37. The method of claim 1, wherein the anti-ICOS antibody comprises a heavy chain comprising the amino acid sequence of SEQ ID NO: 216 and a light chain comprising the amino acid sequence of SEQ ID NO: 189.

38. The method of claim 1, wherein the anti-ICOS antibody consists of a heavy chain having the amino acid sequence of SEQ ID NO: 216 and a light chain having the amino acid sequence of SEQ ID NO: 189.

39. The method of claim 1, wherein administration of the anti-ICOS antibody to a mammal results in an increase in T effector (Teff) cells and/or activation of Teff cells in the mammal.

40. (canceled)

41. (canceled)

42. (canceled)

43. (canceled)

44. The method of claim 1, wherein administration of the antibody to said subject results in a decrease in T regulatory (Treg) cells in said subject.

45. (canceled)

46. The method of claim 1, wherein the subject is a human.

47. The method of claim 1, wherein the method comprises administering an anti-ICOS antibody and at least one additional therapeutic agent.

48. The method of claim 47, wherein the additional therapeutic agent is administered concurrently or sequentially with the anti-ICOS antibody.

49. The method of claim 47, wherein the additional therapeutic agent is selected from an anti-PD-1 antibody and an anti-PD-L1 antibody.

50. (canceled)

51. (canceled)

52. (canceled)

53. The method of claim 47, wherein the additional therapeutic agent is an anti-CTLA4 antibody.

54. The method of claim 53, wherein the anti-CTLA4 antibody is ipilimumab or tremelimumab.

55. The method of claim 47, wherein the additional therapeutic agent is a cancer vaccine.

56. The method of claim 55, wherein the cancer vaccine is selected from a DNA vaccine, an engineered virus vaccine, an engineered tumor cell vaccine, and a cancer vaccine developed using neoantigens.

57. An isolated anti-ICOS antibody, wherein said antibody comprises a heavy chain comprising the amino acid sequence of SEQ ID NO: 216 and a light chain comprising the amino acid sequence of SEQ ID NO: 189.

58. The isolated anti-ICOS antibody of claim 57, wherein said antibody comprises a heavy chain consisting of the amino acid sequence of SEQ ID NO: 216 and a light chain consisting of the amino acid sequence of SEQ ID NO: 189.


SEQUENCE LISTING (216 sequences)

SEQ ID NO: 1 - protein, 199 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 2 - protein, 179 aa, Homo sapiens
EINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 3 - protein, 200 aa, Mus musculus
MKPYFCRVFVFCFLIRLLTGEINGSADHRMFSFHNGGVQISCKYPETVQQLKMRLFREREVLCELTKTKGSGNAVSIKNPMLCLYHLSNNSVSFFLNNPDSSQGSYYFCSLSIFDPPPFQERNLSGGYLHIYESQLCCQLKLWLPVGCAAFVVVLLFGCILIIWFSKKKYGSSVHDPNSEYMFMAAVNTNKKSRLAGVTS

SEQ ID NO: 4 - protein, 180 aa, Mus musculus
EINGSADHRMFSFHNGGVQISCKYPETVQQLKMRLFREREVLCELTKTKGSGNAVSIKNPMLCLYHLSNNSVSFFLNNPDSSQGSYYFCSLSIFDPPPFQERNLSGGYLHIYESQLCCQLKLWLPVGCAAFVVVLLFGCILIIWFSKKKYGSSVHDPNSEYMFMAAVNTNKKSRLAGVTS

SEQ ID NO: 5 - protein, 199 aa, Macaca fascicularis
MKSGLWYFFLFCLHMKVLTGEINGSANYEMFIFHNGGVQILCKYPDIVQQFKMQLLKGGQILCDLTKTKGSGNKVSIKSLKFCHSQLSNNSVSFFLYNLDRSHANYYFCNLSIFDPPPFKVTLTGGYLHIYESQLCCQLKFWLPIGCATFVVVCIFGCILICWLTKKKYSSTVHDPNGEYMFMRAVNTAKKSRLTGTTP

SEQ ID NO: 6 - protein, 179 aa, Macaca fascicularis
EINGSANYEMFIFHNGGVQILCKYPDIVQQFKMQLLKGGQILCDLTKTKGSGNKVSIKSLKFCHSQLSNNSVSFFLYNLDRSHANYYFCNLSIFDPPPFKVTLTGGYLHIYESQLCCQLKFWLPIGCATFVVVCIFGCILICWLTKKKYSSTVHDPNGEYMFMRAVNTAKKSRLTGTTP

SEQ ID NO: 7-19 - skipped (13 entries)

SEQ ID NO: 20 - protein, 117 aa, Mesocricetus auratus
EVQLVESGGGLVKPGGSLKLSCAASGFTFSDYWMDWVRQAPGKGLEWVGNIDEDGSITEYSPFVKGRFTISRDNVKNTLYLQMNSVKSEDTATYYCTRWGRFGFDSWGQGTLVTVSS

SEQ ID NO: 21 - protein, 111 aa, Mesocricetus auratus
DIVMTQSPSSLAVSAGDRVTINCKSSQSLLSGSFNYLTWYQQKTGQAPKLLIFYASTRHTGVPDRFMGSGSGTDFTLTINSFQTEDLGDYYCHHHYNAPPTFGPGTKLELR

SEQ ID NO: 22 - protein, 10 aa, Mesocricetus auratus
GFTFSDYWMD

SEQ ID NO: 23 - protein, 17 aa, Mesocricetus auratus
NIDEDGSITEYSPFVKG

SEQ ID NO: 24 - protein, 8 aa, Mesocricetus auratus
WGRFGFDS

SEQ ID NO: 25 - protein, 15 aa, Mesocricetus auratus
KSSQSLLSGSFNYLT

SEQ ID NO: 26 - protein, 7 aa, Mesocricetus auratus
YASTRHT

SEQ ID NO: 27 - protein, 9 aa, Mesocricetus auratus
HHHYNAPPT

SEQ ID NO: 28-59 - skipped (32 entries)

SEQ ID NO: 60 - protein, 117 aa, Artificial Sequence
  37A10S713 heavy chain variable region
EVQLVESGGGLVQPGGSLRLSCAASGFTFSDYWMDWVRQAPGKGLVWVSNIDEDGSITEYSPFVKGRFTISRDNAKNTLYLQMNSLRAEDTAVYYCTRWGRFGFDSWGQGTLVTVSS

SEQ ID NO: 61 - protein, 111 aa, Artificial Sequence
  37A10S713 light chain variable region
DIVMTQSPDSLAVSLGERATINCKSSQSLLSGSFNYLTWYQQKPGQPPKLLIFYASTRHTGVPDRFSGSGSGTDFTLTISSLQAEDVAVYYCHHHYNAPPTFGPGTKVDIK

SEQ ID NO: 62 - protein, 10 aa, Artificial Sequence
  37A10S713 VH CDR1
GFTFSDYWMD

SEQ ID NO: 63 - protein, 17 aa, Artificial Sequence
  37A10S713 VH CDR2
NIDEDGSITEYSPFVKG

SEQ ID NO: 64 - protein, 8 aa, Artificial Sequence
  37A10S713 VH CDR3
WGRFGFDS

SEQ ID NO: 65 - protein, 15 aa, Artificial Sequence
  37A10S713 VL CDR1
KSSQSLLSGSFNYLT

SEQ ID NO: 66 - protein, 7 aa, Artificial Sequence
  37A10S713 VL CDR2
YASTRHT

SEQ ID NO: 67 - protein, 9 aa, Artificial Sequence
  37A10S713 VL CDR3
HHHYNAPPT

SEQ ID NO: 68-69 - skipped (2 entries)

SEQ ID NO: 70 - protein, 117 aa, Artificial Sequence; identical to SEQ ID NO: 60
  37A10S714 heavy chain variable region

SEQ ID NO: 71 - protein, 111 aa, Artificial Sequence
  37A10S714 light chain variable region
DIVMTQSPDSLAVSLGERATINCKSSQSLLSGSFNYLTWYQQKPGQPPKLLIFYASTRETGVPDRFSGSGSGTDFTLTISSLQAEDVAVYYCHHHYNAPPTFGPGTKVDIK

SEQ ID NO: 72 - protein, 10 aa, Artificial Sequence
  37A10S714 VH CDR1
GFTFSDYWMD

SEQ ID NO: 73 - protein, 17 aa, Artificial Sequence
  37A10S714 VH CDR2
NIDEDGSITEYSPFVKG

SEQ ID NO: 74 - protein, 8 aa, Artificial Sequence
  37A10S714 VH CDR3
WGRFGFDS

SEQ ID NO: 75 - protein, 15 aa, Artificial Sequence
  37A10S714 VL CDR1
KSSQSLLSGSFNYLT

SEQ ID NO: 76 - protein, 7 aa, Artificial Sequence
  37A10S714 VL CDR2
YASTRET

SEQ ID NO: 77 - protein, 9 aa, Artificial Sequence
  37A10S714 VL CDR3
HHHYNAPPT

SEQ ID NO: 78-79 - skipped (2 entries)

SEQ ID NO: 80 - protein, 117 aa, Artificial Sequence; identical to SEQ ID NO: 60
  37A10S715 heavy chain variable region

SEQ ID NO: 81 - protein, 111 aa, Artificial Sequence
  37A10S715 light chain variable region
DIVMTQSPDSLAVSLGERATINCKSSQSLLSGSFNYLTWYQQKPGQPPKLLIFYASTRQTGVPDRFSGSGSGTDFTLTISSLQAEDVAVYYCHHHYNAPPTFGPGTKVDIK

SEQ ID NO: 82 - protein, 10 aa, Artificial Sequence
  37A10S715 VH CDR1
GFTFSDYWMD

SEQ ID NO: 83 - protein, 17 aa, Artificial Sequence
  37A10S715 VH CDR2
NIDEDGSITEYSPFVKG

SEQ ID NO: 84 - protein, 8 aa, Artificial Sequence
  37A10S715 VH CDR3
WGRFGFDS

SEQ ID NO: 85 - protein, 15 aa, Artificial Sequence
  37A10S715 VL CDR1
KSSQSLLSGSFNYLT

SEQ ID NO: 86 - protein, 7 aa, Artificial Sequence
  37A10S715 VL CDR2
YASTRQT

SEQ ID NO: 87 - protein, 9 aa, Artificial Sequence
  37A10S715 VL CDR3
HHHYNAPPT

SEQ ID NO: 88-89 - skipped (2 entries)

SEQ ID NO: 90 - protein, 117 aa, Artificial Sequence
  37A10S716 heavy chain variable region
EVQLVESGGGLVQPGGSLRLSCAASGFTFSDYWMDWVRQAPGKGLVWVSNIDESGSITEYSPFVKGRFTISRDNAKNTLYLQMNSLRAEDTAVYYCTRWGRFGFDSWGQGTLVTVSS

SEQ ID NO: 91 - protein, 111 aa, Artificial Sequence; identical to SEQ ID NO: 61
  37A10S716 light chain variable region

SEQ ID NO: 92 - protein, 10 aa, Artificial Sequence
  37A10S716 VH CDR1
GFTFSDYWMD

SEQ ID NO: 93 - protein, 17 aa, Artificial Sequence
  37A10S716 VH CDR2
NIDESGSITEYSPFVKG

SEQ ID NO: 94 - protein, 8 aa, Artificial Sequence
  37A10S716 VH CDR3
WGRFGFDS

SEQ ID NO: 95 - protein, 15 aa, Artificial Sequence
  37A10S716 VL CDR1
KSSQSLLSGSFNYLT

SEQ ID NO: 96 - protein, 7 aa, Artificial Sequence
  37A10S716 VL CDR2
YASTRHT

SEQ ID NO: 97 - protein, 9 aa, Artificial Sequence
  37A10S716 VL CDR3
HHHYNAPPT

SEQ ID NO: 98-99 - skipped (2 entries)

SEQ ID NO: 100 - protein, 117 aa, Artificial Sequence; identical to SEQ ID NO: 90
  37A10S717 heavy chain variable region

SEQ ID NO: 101 - protein, 111 aa, Artificial Sequence; identical to SEQ ID NO: 71
  37A10S717 light chain variable region

SEQ ID NO: 102 - protein, 10 aa, Artificial Sequence
  37A10S717 VH CDR1
GFTFSDYWMD

SEQ ID NO: 103 - protein, 17 aa, Artificial Sequence
  37A10S717 VH CDR2
NIDESGSITEYSPFVKG

SEQ ID NO: 104 - protein, 8 aa, Artificial Sequence
  37A10S717 VH CDR3
WGRFGFDS

SEQ ID NO: 105 - protein, 15 aa, Artificial Sequence
  37A10S717 VL CDR1
KSSQSLLSGSFNYLT

SEQ ID NO: 106 - protein, 7 aa, Artificial Sequence
  37A10S717 VL CDR2
YASTRET

SEQ ID NO: 107 - protein, 9 aa, Artificial Sequence
  37A10S717 VL CDR3
HHHYNAPPT

SEQ ID NO: 108-109 - skipped (2 entries)

SEQ ID NO: 110 - protein, 117 aa, Artificial Sequence; identical to SEQ ID NO: 90
  37A10S718 heavy chain variable region

SEQ ID NO: 111 - protein, 111 aa, Artificial Sequence; identical to SEQ ID NO: 81
  37A10S718 light chain variable region

SEQ ID NO: 112 - protein, 10 aa, Artificial Sequence
  37A10S718 VH CDR1
GFTFSDYWMD

SEQ ID NO: 113 - protein, 17 aa, Artificial Sequence
  37A10S718 VH CDR2
NIDESGSITEYSPFVKG

SEQ ID NO: 114 - protein, 8 aa, Artificial Sequence
  37A10S718 VH CDR3
WGRFGFDS

SEQ ID NO: 115 - protein, 15 aa, Artificial Sequence
  37A10S718 VL CDR1
KSSQSLLSGSFNYLT

SEQ ID NO: 116 - protein, 7 aa, Artificial Sequence
  37A10S718 VL CDR2
YASTRQT

SEQ ID NO: 117 - protein, 9 aa, Artificial Sequence
  37A10S718 VL CDR3
HHHYNAPPT

SEQ ID NO: 118-187 - skipped (70 entries)

SEQ ID NO: 188 - protein, 447 aa, Artificial Sequence
  37A10S713 human IgG1 heavy chain
EVQLVESGGGLVQPGGSLRLSCAASGFTFSDYWMDWVRQAPGKGLVWVSNIDEDGSITEYSPFVKGRFTISRDNAKNTLYLQMNSLRAEDTAVYYCTRWGRFGFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 189 - protein, 219 aa, Artificial Sequenc
  37A10S713 human kappa light chain
EDIVMTQSPDSLAVSLGERATINCKSSQSLLSGSFNYLTWYQQKPGQPPKLLIFYASTRHTGVPDRFSGSGSGTDFTLTISSLQAEDVAVYYCHHHYNAPPTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 190 - protein, 200 aa, Rattus norvegicus
MKPYFSCVFVFCFLIKLLTGELNDLANHRMFSFHDGGVQISCNYPETVQQLKMQLFKDREVLCDLTKTKGSGNTVSIKNPMSCPYQLSNNSVSFFLDNADSSQGSYFLCSLSIFDPPPFQEKNLSGGYLLIYESQLCCQLKLWLPVGCAAFVAALLFGCIFIVWFAKKKYRSSVHDPNSEYMFMAAVNTNKKSRLAGMTS

SEQ ID NO: 191 - protein, 180 aa, Rattus norvegicus
ELNDLANHRMFSFHDGGVQISCNYPETVQQLKMQLFKDREVLCDLTKTKGSGNTVSIKNPMSCPYQLSNNSVSFFLDNADSSQGSYFLCSLSIFDPPPFQEKNLSGGYLLIYESQLCCQLKLWLPVGCAAFVAALLFGCIFIVWFAKKKYRSSVHDPNSEYMFMAAVNTNKKSRLAGMTS

SEQ ID NO: 192 - protein, 120 aa, Artificial Sequence
  2M13 heavy chain variable region
EVQLQQSGAELVRPGAVVKLSCKASGFDIKDYYMHWVQQRPEQGLEWIGWIDPENGNAVYDPQFQGKASITADTSSNTAYLQLSSLTSEDTAVYYCASDYYGSKGYLDVWGAGTTVTVSS

SEQ ID NO: 193 - protein, 106 aa, Artificial Sequence
  2M13 light chain variable region
QIVLTQSPTIMSASPGEKVTITCSASSSVSYMHWFQQKPGTSPKLWIYSTSNLASGVPARFGGSRSGTSYSLTISRMEAEDAATYYCQQRSSYPFTFGSGTKLEIK

SEQ ID NO: 194 - protein, 5 aa, Artificial Sequence
  2M13 VH CDR1
DYYMH

SEQ ID NO: 195 - protein, 17 aa, Artificial Sequence
  2M13 VH CDR2
WIDPENGNAVYDPQFQG

SEQ ID NO: 196 - protein, 11 aa, Artificial Sequence
  2M13 VH CDR3
DYYGSKGYLDV

SEQ ID NO: 197 - protein, 10 aa, Artificial Sequence
  2M13 VL CDR1
SASSSVSYMH

SEQ ID NO: 198 - protein, 7 aa, Artificial Sequence
  2M13 VL CDR2
STSNLAS

SEQ ID NO: 199 - protein, 9 aa, Artificial Sequence
  2M13 VL CDR3
QQRSSYPFT

SEQ ID NO: 200 - protein, 115 aa, Artificial Sequence
  2M19 heavy chain variable region
EVQLQQSGAELVRSGASVKLSCTTSAFNIIDYYMHWVIQRPEQGLEWIAWIDPENGDPEYAPKFQDKATMTTDTSSNTAYLQLSSLTSEDTAVYYCTAWRGFAYWGQGTLVTVSA

SEQ ID NO: 201 - protein, 112 aa, Artificial Sequence
  2M19 light chain variable region
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSIHVPPTFGGGTKLEIK

SEQ ID NO: 202 - protein, 5 aa, Artificial Sequence
  2M19 VH CDR1
DYYMH

SEQ ID NO: 203 - protein, 17 aa, Artificial Sequence
  2M19 VH CDR2
WIDPENGDPEYAPKFQD

SEQ ID NO: 204 - protein, 6 aa, Artificial Sequence
  2M19 VH CDR3
WRGFAY

SEQ ID NO: 205 - protein, 16 aa, Artificial Sequence
  2M19 VL CDR1
RSSQSLVHSNGNTYLH

SEQ ID NO: 206 - protein, 7 aa, Artificial Sequence
  2M19 VL CDR2
KVSNRFS

SEQ ID NO: 207 - protein, 9 aa, Artificial Sequence
  2M19 VL CDR3
SQSIHVPPT

SEQ ID NO: 208 - protein, 115 aa, Artificial Sequence
  2M24 heavy chain variable region
EVQLQQSGAELVRSGASVKLSCTASGFNIRDYYMHWVRQRPEQGLEWIGWIDPENGDIDYAPKFQDKATMTADTSSNTAYLQLSSLTSEDSAVYYFTAWKGLAYWGQGTLVTVSA

SEQ ID NO: 209 - protein, 112 aa, Artificial Sequence
  2M24 light chain variable region
DVVMTQTPLSLPVSLGDQASMSCRSSQSLVHSNGNTYLQWYLQKPGQSPKLLIYKVFNRFSGVPDRFSGSGSGTDFTLKISRVEAEDLGVYFCSQSTHVPPTFGGGTKLEIK

SEQ ID NO: 210 - protein, 5 aa, Artificial Sequence
  2M24 VH CDR1
DYYMH

SEQ ID NO: 211 - protein, 17 aa, Artificial Sequence
  2M24 VH CDR2
WIDPENGDIDYAPKFQD

SEQ ID NO: 212 - protein, 6 aa, Artificial Sequence
  2M24 VH CDR3
WKGLAY

SEQ ID NO: 213 - protein, 16 aa, Artificial Sequence
  2M24 VL CDR1
RSSQSLVHSNGNTYLQ

SEQ ID NO: 214 - protein, 7 aa, Artificial Sequence
  2M24 VL CDR2
KVFNRFS

SEQ ID NO: 215 - protein, 9 aa, Artificial Sequence
  2M24 VL CDR3
SQSTHVPPT

SEQ ID NO: 216 - protein, 446 aa, Artificial Sequence
  37A10S713 human IgG1 heavy chain V2
EVQLVESGGGLVQPGGSLRLSCAASGFTFSDYWMDWVRQAPGKGLVWVSNIDEDGSITEYSPFVKGRFTISRDNAKNTLYLQMNSLRAEDTAVYYCTRWGRFGFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG